(19) United States
(12) Patent Application Publication
(10) Pub. No.: US 2006/0088835 A1
(43) Pub. Date: Apr. 27, 2006

(54) Schizophrenia associated genes

(76) Inventors: Benjamin Simon Pickard, Edinburgh (GB); Douglas Blackwood, Edinburgh (GB); David John Porteous, Edinburgh (GB); Walter John Muir, Tweeddale (GB); Ole Mors, Risskov (DK); Henrik Lykke Ewald, Hobro (DK)

Correspondence Address: MYERS BIGEL SIBLEY & SAJOVEC, PO BOX 37428, RALEIGH, NC, 27627, US

(21) Appl. No.: 10/511,455

(22) PCT Filed: Apr. 7, 2003
(86) PCT No.: PCT/GB03/01543

§ 371 Date: Aug. 29, 2005

(30) Foreign Application Priority Data
Apr. 5, 2002 (GB) 0207902.8
Apr. 5, 2002 (GB) 0207904.4
Apr. 5, 2002 (GB) 0207900.2
Apr. 5, 2002 (GB) 0207901.0
Nov. 28, 2002 (GB) 0227734.1
Nov. 28, 2002 (GB) 0227733.3

(51) Int. Cl. A61K 48/00; A01K 67/00; C12Q 1/68

(52) U.S. Cl. 435/6; 514/44; 800/8

(57) ABSTRACT

The present invention relates to the identification of genes which have been disrupted in patients diagnosed as suffering from schizophrenia and/or bi-polar affective disorder, as well as proteins encoded by the gene and antibodies thereto and to uses of such products as medicaments for treating schizophrenia and/or affective psychosis. The invention also relates to methods for diagnosing, patients suffering or predisposed to schizophrenia and/or affective psychosis, as well as screens for developing novel treatment regimes for schizophrenia and/or affective psychosis.

The present invention relates to the identification of genes which have been disrupted in patients diagnosed as suffering from schizophrenia and/or bi-polar affective disorder, as well as proteins encoded by the gene and antibodies thereto and to uses of such products as medicaments for treating schizophrenia and/or affective psychosis. The invention also relates to methods for diagnosing patients suffering or predisposed to schizophrenia and/or affective psychosis, as well as screens for developing novel treatment regimes for schizophrenia and/or affective psychosis.

Schizophrenia and Bipolar Affective Disorder are common and debilitating psychiatric disorders. Despite a wealth of information on the epidemiology, neuroanatomy and pharmacology of the illness, it is uncertain what molecular pathways are involved and how impairments in these affect brain development and neuronal function. Despite an estimated heritability of 60-80%, very little is known about the number or identity of genes involved in these psychoses. Although there has been recent progress in linkage and association studies, especially from genome-wide scans, these studies have yet to progress from the identification of susceptibility loci or candidate genes to the full characterisation of disease-causing genes (Berrettini, 2000).

The cloning of breakpoints in patients with chromosome abnormalities (translocations, inversions etc.) has proved instrumental in the identification of many disease genes (e.g. Duchenne Muscular Dystrophy, Retinoblastoma, Wilm's Tumour, Familial Polyposis Coli, Fragile-X Syndrome, Polycystic Kidney Disease, many leukaemias and, very recently, a candidate speech and language disorder gene (Lai et al, 2001)). Such studies assume that the chromosomal breakpoints give rise to the clinical symptoms by either directly disrupting gene sequences or perturbing gene expression. In the same way that gene-trap mutagenesis can be used to identify disrupted mouse genes (Brennan & Skarnes, 1999), the physical “flag” created by a cytogenetic breakpoint provides a geographical pointer for the disease locus.

It is amongst the objects of the present invention to provide genes and/or proteins postulated to be involved with the development and/or symptoms associated with schizophrenia and/or affective psychosis.

As will be seen, the present invention is based on the molecular characterisation of a chromosomal disruption in subjects diagnosed as suffering from a schizophrenia and/or affective psychosis. A high-throughput Fluorescence in situ Hybridisation (FISH)-based approach has been adopted to map the chromosomal breakpoints in these patients. Consultation of the sequence data at the breakpoint locus not only allows efficient FISH probe selections to be made by the targeting of coding regions, but also proof of gene disruption can be made entirely by relating the exact position of probes to the genomic structure of a candidate gene.

Four patients have been studied and their chromosomal disruptions characterised. Hereinafter the patients will be identified as patients 1-4.

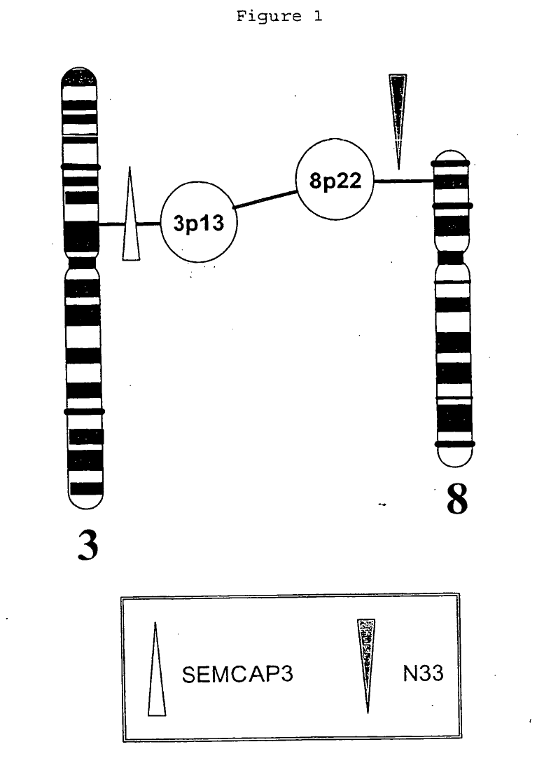

As will be seen, in one embodiment, the present invention is based on the molecular characterisation of a chromosomal rearrangement denoted t(3;8) (p13;p22) in a subject (patient 1) diagnosed as suffering from a schizoaffective disorder (see FIG. 1). A high-throughput Fluorescence in situ Hybridisation (FISH)-based approach was adopted to map the chromosomal breakpoints in these patients. Consultation of the sequence data at the breakpoint loci not only allowed efficient FISH probe selections to be made by the targeting of coding regions, but also proof of gene disruption was inferred entirely by relating the exact position of probes to the genomic structure of a candidate gene.

One breakpoint (located on chromosome 8p22) in this subject lies near to a gene, N33, involved in the N-Linked Glycosylation pathway.

This pathway consists of three stages. Firstly the assembly of a donor oligosaccharide at the endoplasmic reticulum lumen membrane. Secondly, the transfer of this molecule onto newly translated secretory and transmembrane proteins catalyzed by the oligosaccharyltransferase (OST) complex. Thirdly, there is subsequent modification of the oligosaccharides on the glycoprotein. N33 encodes a protein thought to be involved in the second stage of the pathway by analogy with yeast homologues. Without wishing to be bound by theory it is hypothesised that the breakpoint in the subject perturbs N33 expression indirectly through position effect silencing or separation of regulatory elements from the gene promoter (both effects have been shown to occur even when the breakpoints are up to 1 Mb from the target gene in some instances (Kleinjan et al 1999)).

As the N33 gene is located within a chromosomal region repeatedly found positive in schizophrenia linkage studies the present inventors pursued this gene further by association study.

Certain microsatellite repeat haplotypes have been identified at the N33 locus which are over-represented in schizophrenic patients and their families compared to the normal population. Subsequent sequencing of the N33 gene in haplotype carrying individuals is ongoing in order to identify causative mutations.

The other breakpoint in this patient (3p13) has now also been fully characterised and demonstrated to disrupt a gene, SEMCAP3 (also known as KIAA1095). The present invention is therefore also based on a proposed role of this gene (normal and mutated forms) in the aetiology of schizophrenia and/or affective psychosis.

In a further embodiment the present invention is based on the GRIK4 gene and observations of the present inventors of an involvement of this gene and/or protein with schizophrenia and/or affective psychosis.

The GRIK4 gene is also known as KA1 and EAA1, but will herein be referred to as GRIK4 for simplicity, but should not be construed as limiting.

The subject (patient 2) was one of a series of around 100 patients with comorbid schizophrenia and mild learning disability (US terminology: “mental retardation”) who were screened using routine G-band karyotyping. This patient possesses a complex chromosomal rearrangement which can be described by standard nomenclature as; (46, XX, ins(8;11) (q13;q23.3q24.2)inv(2)(p12q32.1)t(2;11)(q21.3;q24.2)der (2) (2qter→2q32.1::2p12→2q21.3::11q24.2→11qter)der(11) (11pter→11q23.3::2q21.3→2q32.1::2p12→2pter)der(8) (8pter→8q13::11q23.2→11q24.2::8q13→8qter)). It has been repeatedly observed that schizophrenia occurs more frequently in individuals with mild learning disability than in the general population and recent work has revealed an increased heritability of this comorbid state.

As described herein the FISH results reveal that the subject has a disruption in a brain expressed gene; namely, GRIK4 which is known to participate in molecular mechanisms responsible for modulating the strength of synaptic transmission.

In a further embodiment the present invention is based on the characterisation of a balanced reciprocal translocation between chromosomes 9 and 14, t(9;14)(q34;q13) in a mother (patient 3) with schizophrenia and her daughter with schizophrenia co-morbid with mild learning disability. A brain transcription factor gene, NPAS3, is shown to be disrupted by the translocation at 14q13. Without wishing to be bound by theory, the present inventors hypothesis is that the disruption of this gene is responsible for the psychotic symptoms exhibited by the mother and daughter.

As will be seen, the present invention is also based on the molecular characterisation of a chromosomal rearrangement denoted t(1;16)(p31.2;q21) (in patient 4).

The proband met ICD-10 and DSM-IV criteria for definite schizophrenia. The translocation was inherited within other branches of the family with variable clinical expression. However some key translocation carriers of the subjects to whom the inventors had access had not passed the age of risk when clinically characterized.

One breakpoint (located on chromosome 1p31.2) in patient 4 lies within an alternatively spliced form of the gene, PDE4B, involved in the attenuation of cAMP secondary messenger signaling.

The remaining breakpoint in this patient (16q21) has now also fully characterised and demonstrated to disrupt a gene, CADHERIN 8 (CDH8). The present invention is therefore based in part on a proposed role of this gene in the aetiology of schizophrenia and/or affective psychosis.

In a first aspect the present invention provides use of a polynucleotide fragment or fragments comprising SEMCAP3, N33, NPAS3, GRIK4, PDE4B and/or CDH8 gene(s) or a fragment(s), derivative(s) or homologue(s) thereof for the manufacture of a medicament for treating schizophrenia and/or affective psychosis in a subject.

In another aspect the present invention provides use of a polypeptide fragment or fragments encoded by SEMCAP3, N33, NPAS3, GRIK4, PDE4B and/or CDH8 gene(s), or a fragment(s), derivative(s) or homologue(s) thereof for the manufacture of a medicament for treating schizophrenia and/or affective psychosis in a subject.

Schizophrenia and/or affective psychosis as used herein relates to schizophrenia, as well as other affective psychoses such as those listed in “The ICD-10 Classification of Mental and Behavioural Disorders” World Health Organization, Geneva 1992. Categories F20 to F29 inclusive includes Schizophrenia, schizotypal and delusional disorders. Categories F30 to F39 inclusive are Mood (affective) disorders that include bipolar affective disorder and depressive disorder. Mental Retardation is coded F70 to F79 inclusive. The Diagnostic and Statistical Manual of Mental Disorders, Fourth Edition (DSM-IV). American Psychiatric Association, Washington DC. 1994. Include all conditions coded 295.xx (Schizophrenia and Other Psychotic Disorders) and 296.xx (Major Depressive Disorders and Bipolar Disorders). Mental Retardation is coded 315, 317, 318 and 319.

SEMCAP3 has been previously cloned and sequenced in mouse as two alternative forms (Semcap3A and 3B) and the sequences are present in the public database (nucleic acid sequences; AF127084/AF127085, respectively; protein sequences AAF22131/AAF22132, respectively) as directly submitted by Wang & Strittmatter, 1999. The human form of the gene is defined by sequence KIAA1095 (nucleic acid sequence, AB029018 or XM_{—}041363, and a smaller form, BC014432; protein sequence, XP_{—}041363). The genomic sequences corresponding to this gene are also present in the public database (eg. for BAC RP11-252o10, AC024102). Nevertheless, the prior art does not suggest any link between SEMCAP3 and schizophrenia and/or affective psychosis.

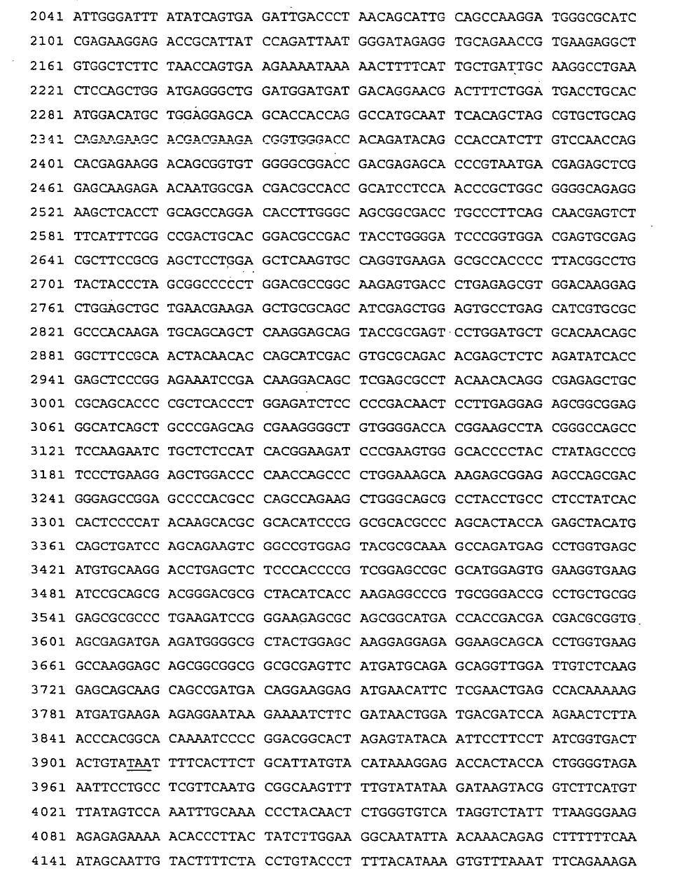

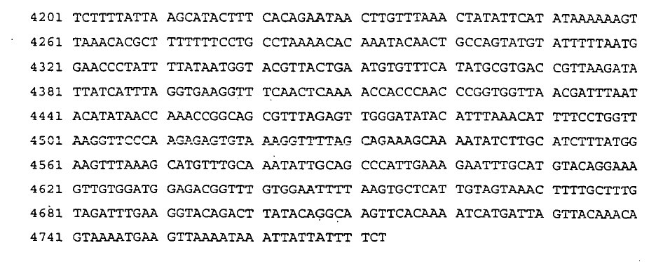

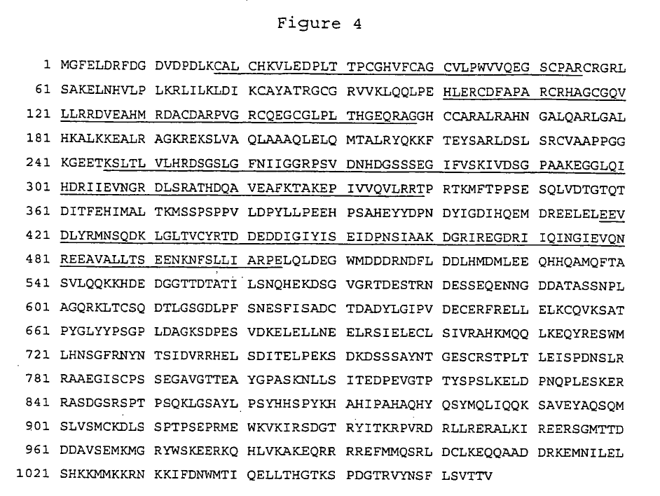

Thus, references herein to the SEMCAP3 gene are understood to relate to the sequences in the public databases and identified in FIG. 3 and references to the SEMCAP3 protein sequence is understood to relate to the sequences in the public databases and identified in FIG. 4.

N33 has been previously cloned and sequenced and the sequence is present in the public database (Nucleic acid-sequence; U42349, Protein sequence; Q13454) and described in MacGrogan et al, 1996. The genomic sequences corresponding to this gene are also present in the public database (eg. for BAC RP11-23j14) but some SNP polymorphisms or sequencing errors (eg. an extra “C” present in exon 1b, see hereinafter—cctgccccaccggg—may result in differences to the sequences presented herein. Nevertheless, the prior art does not suggest any link between N33 and schizophrenia and affective psychosis.

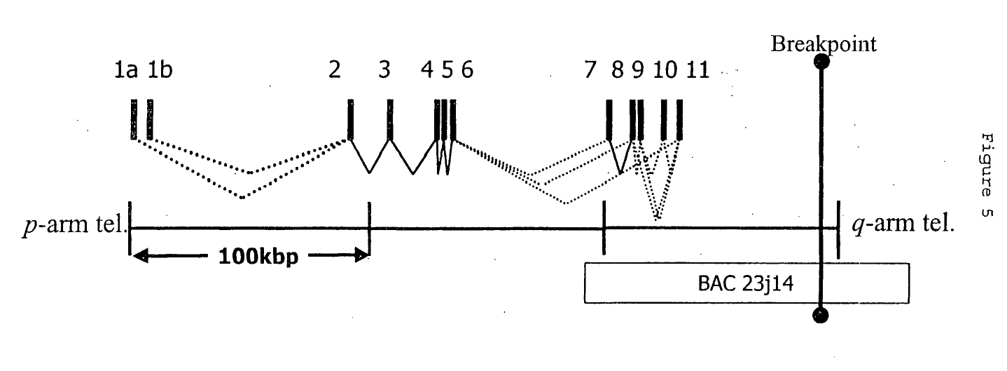

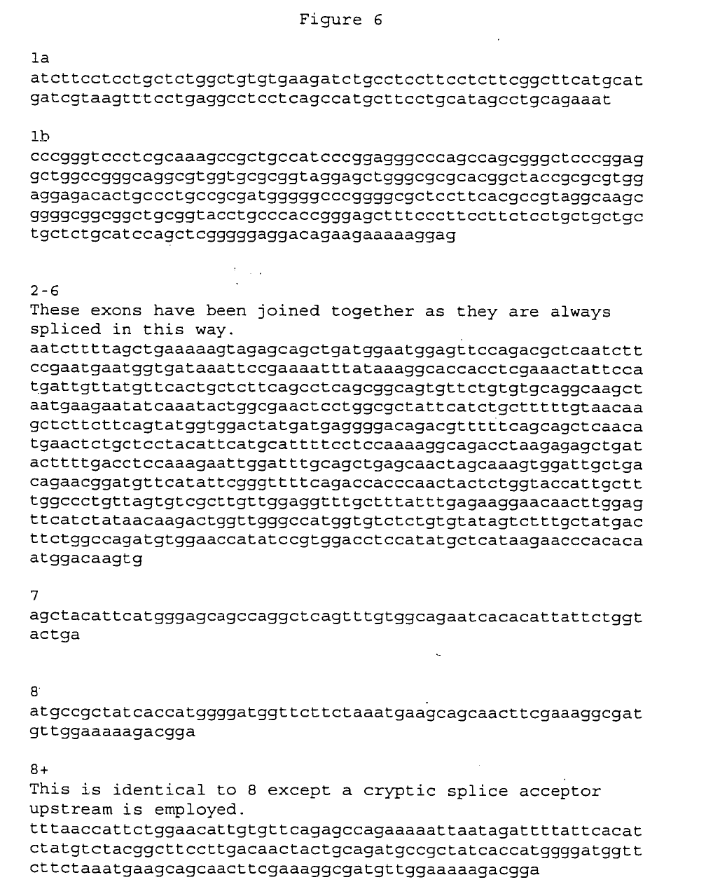

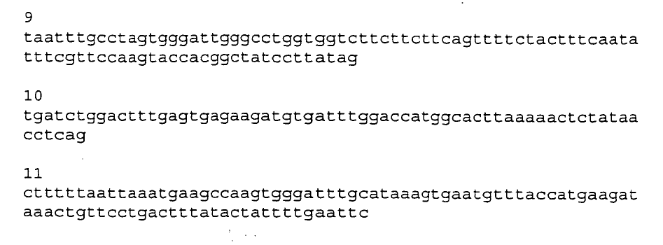

In addition to the sequences previously identified, the present inventors have identified a new start exon (1a, see FIGS. 6 and 7) and have observed the complexity of the exon splicing at the 3′ end of the gene (see FIGS. 6 and 7).

Thus, references herein to the N33 gene are understood to relate to the sequences in the public databases and identified in FIGS. 6 and 7 and references to the N33 protein sequence are understood to relate to the sequences in the public databases and identified in FIGS. 6 and 7.

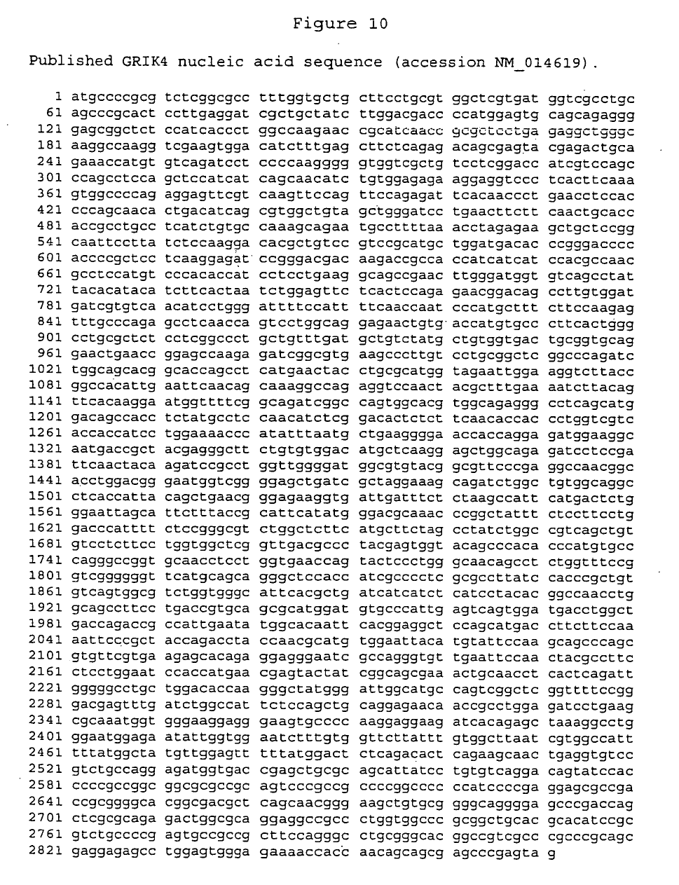

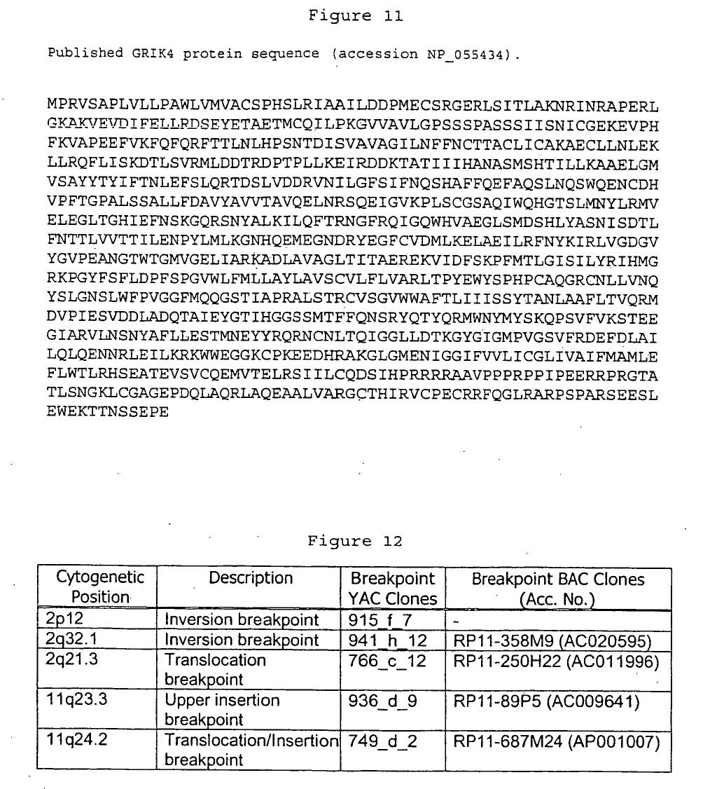

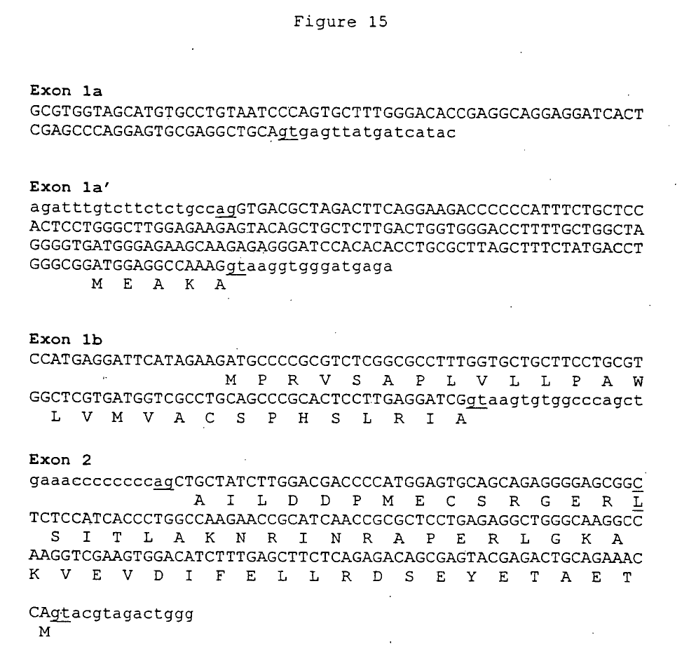

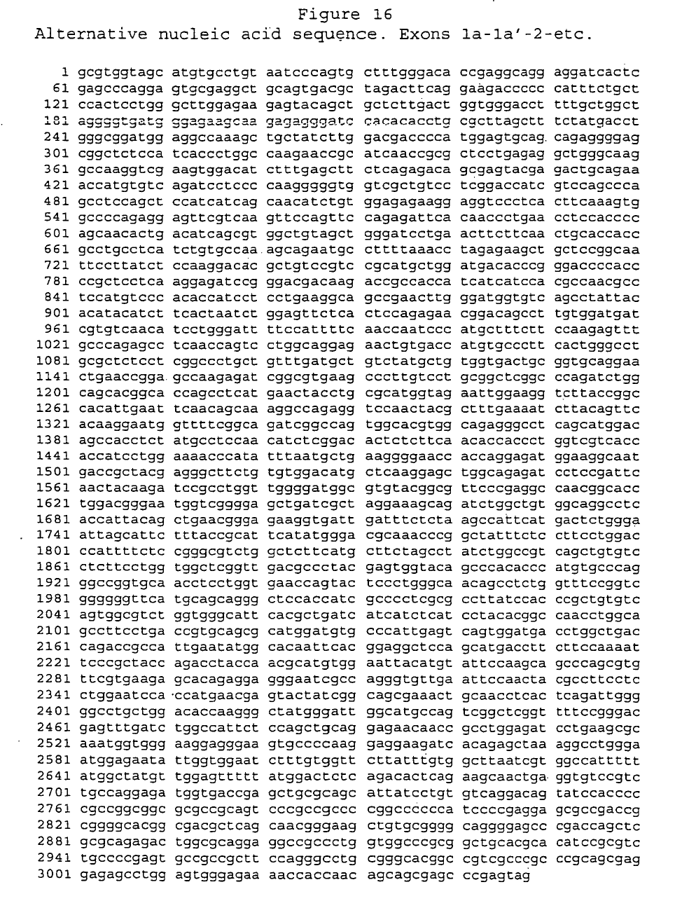

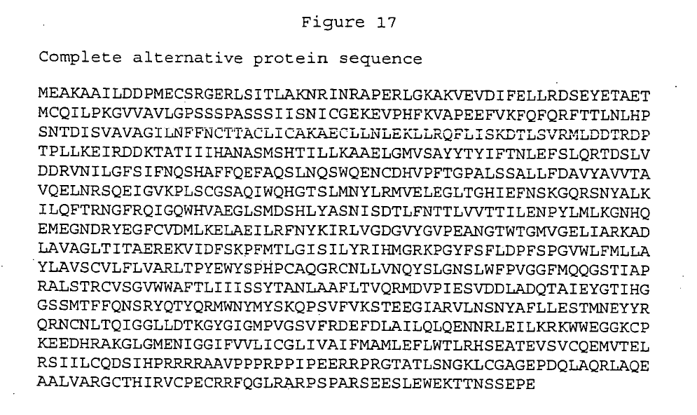

The GRIK4 gene is located on chromosome 11, at cytogenetic position 11q22.3. The gene encodes a kainate receptor subunit and has been previously described by Kamboj et al, 1994. The cDNA nucleotide sequence and peptide sequence was disclosed by Kamboj et al, 1994 and submitted to the Genbank/EMBL database under accession NM_{—}014619. The coding sequence of the gene is identified as being 2871 nucleotides in length, coding for a protein 957 amino acids. The nucleotide and protein sequences are shown in FIGS. 10 and 11 respectively. The present inventors have identified an alternative start site for the gene (see FIGS. 15-17) which would result in a shorter gene/protein of 933 amino acids as opposed to 956. The full nucleotide sequence and protein sequence of this alternatively encoded gene/protein is shown in FIGS. 16 and 17.

Thus, references herein to the GRIK4 gene are understood to relate to the sequences identified in FIGS. 10 and 16 and references to the GRIK4 protein sequence are understood to relate to the sequences identified in FIGS. 11 and 17.

The human form of NPAS3 has previously been identified and is found in the public database under accession numbers AB054575 and AF164438, with the differences due to alternative splicing and all forms are encompassed within the present invention.

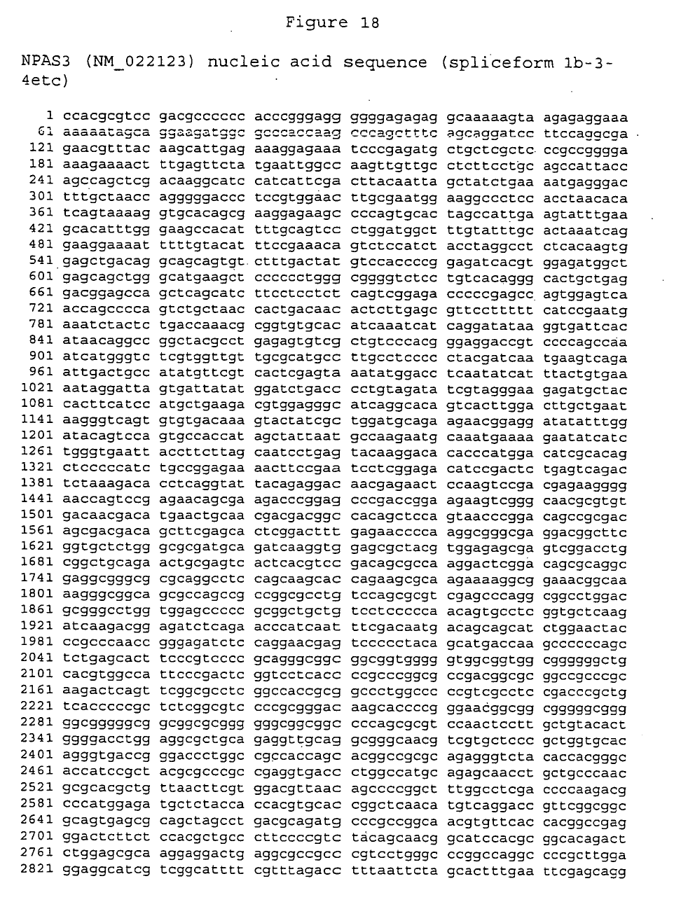

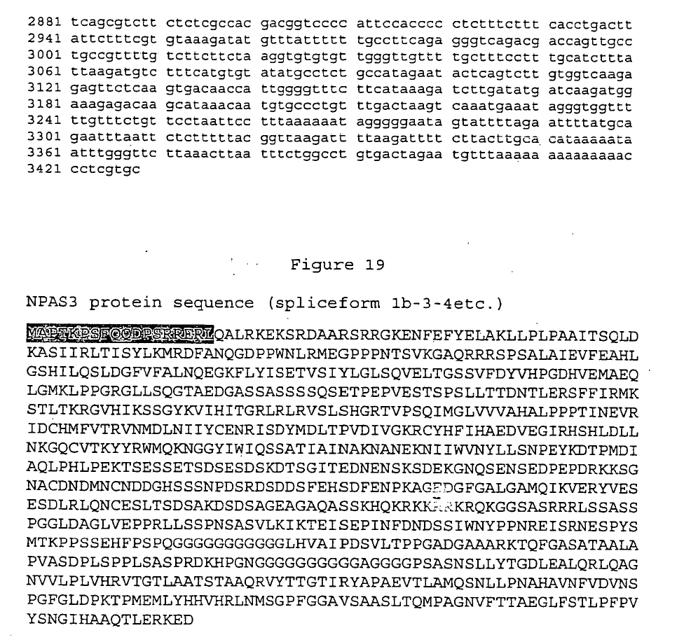

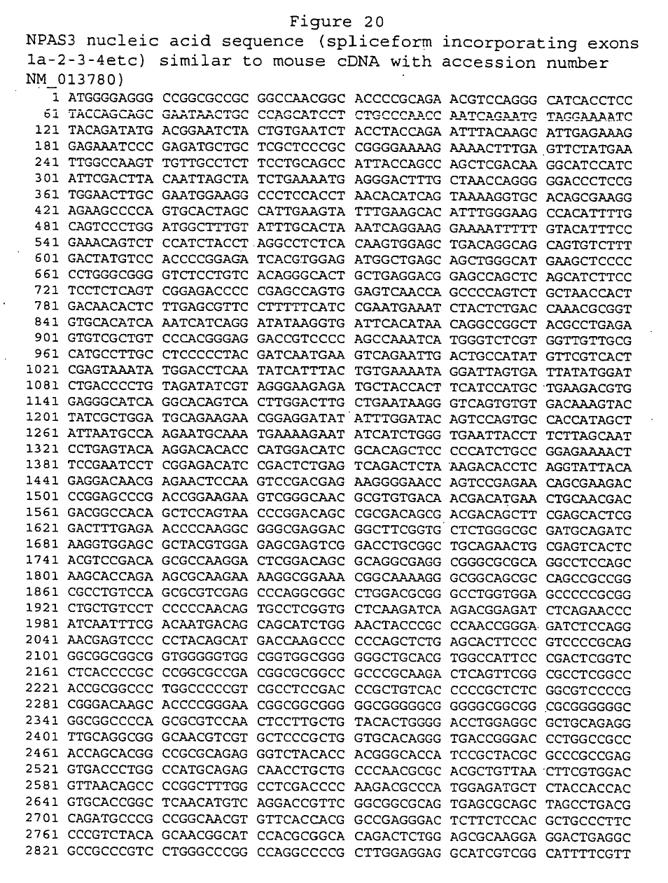

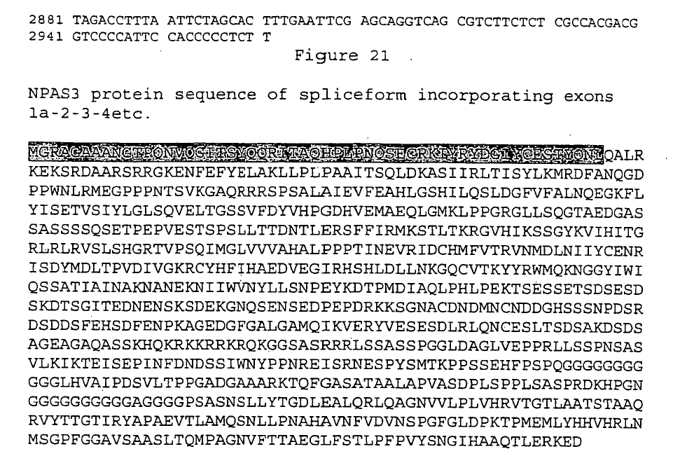

Thus, references herein to the NPAS3 gene are understood to relate to the sequences identified in FIGS. 18 and 20 and references to the NPAS3 protein sequence are understood to relate to the sequences identified in FIGS. 19 and 21.

The PDE4B gene is located on chromosome 1 at cytogenetic position 1p31.2. The gene encodes a phosphodiesterase which shows homology to the Dunce leaning and memory gene product of Drosophila melanogaster, Bolger et al, 1993. Two long (PDE4B1 and PDE4B3) and one short (PDE4B2) splice form are described herein. There is a core protein sequence of 525 amino acid residues shared by all three forms. On to this is added 39 N-terminal amino acid residues in the case of PDE4B2. Both of the long forms share an additional central stretch of 118 amino acid residues, but then diverge at the N-terminal end of the proteins; PDE4B1 has 93 specific residues and PDE4B3, 78. It is predicted that only the PDE4B1 splice form (brain expressed) may be disrupted by the chromosomal abnormality observed in the patient and family.

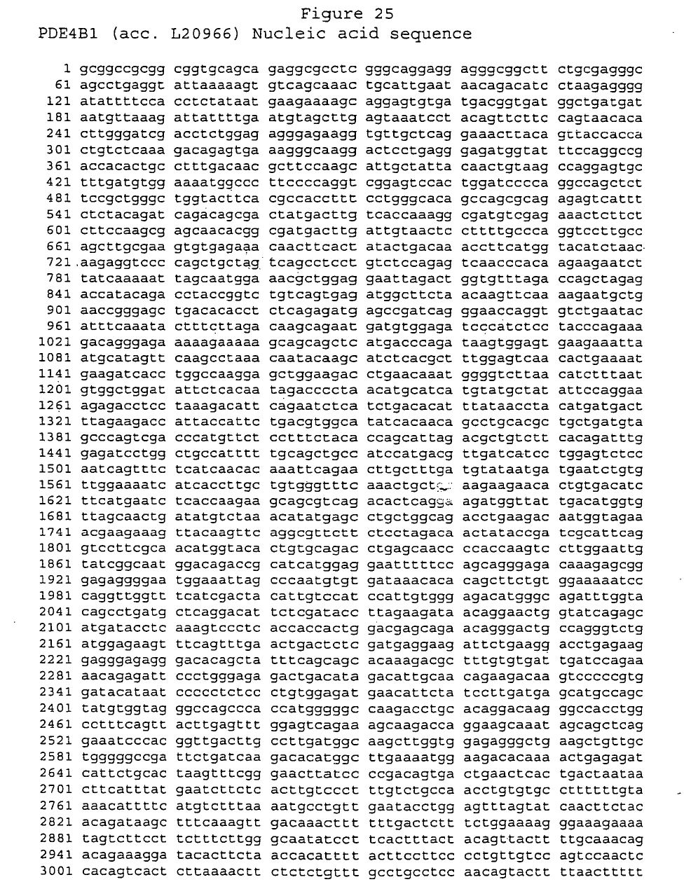

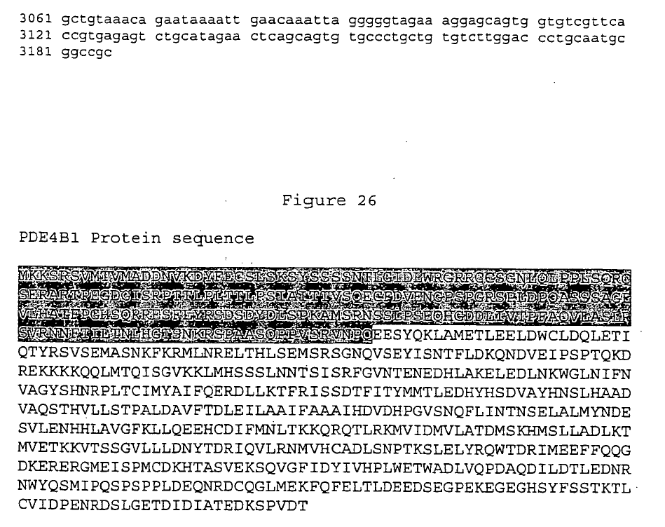

Thus, references herein to the PDE4B gene are understood to relate to the sequences identified in FIGS. 25, 27 and 29 and references to the PDE4B protein sequence are understood to relate to the sequences identified in FIGS. 26, 28 and 30.

CADHERIN 8 (CDH8) has been previously cloned and sequenced and the sequence is present in the public database (nucleic acid sequence; L34060/AB035305/NM_{—}001796, protein sequence; NP_{—}001787) and described in Suzuki et al., 1991, Tanihara et al., 1994, and Shimoyama et al., 2000. An alternative transcript form has been described in the rat in which there is a truncation within the 5^{th} cadherin domain (Kido et al., 1998 and see FIG. 4). The accession numbers for the normal and truncated forms of CDH8 in rat are AB010436 and AB010437, respectively. The corresponding human truncated transcript is not present in the public database and so is not yet confirmed. The genomic sequences corresponding to CDHB are also present in the public database (eg. BAC CTC-420A11; AC040161). Nevertheless, the prior art does not suggest any link between CDH8 and schizophrenia and/or affective psychosis.

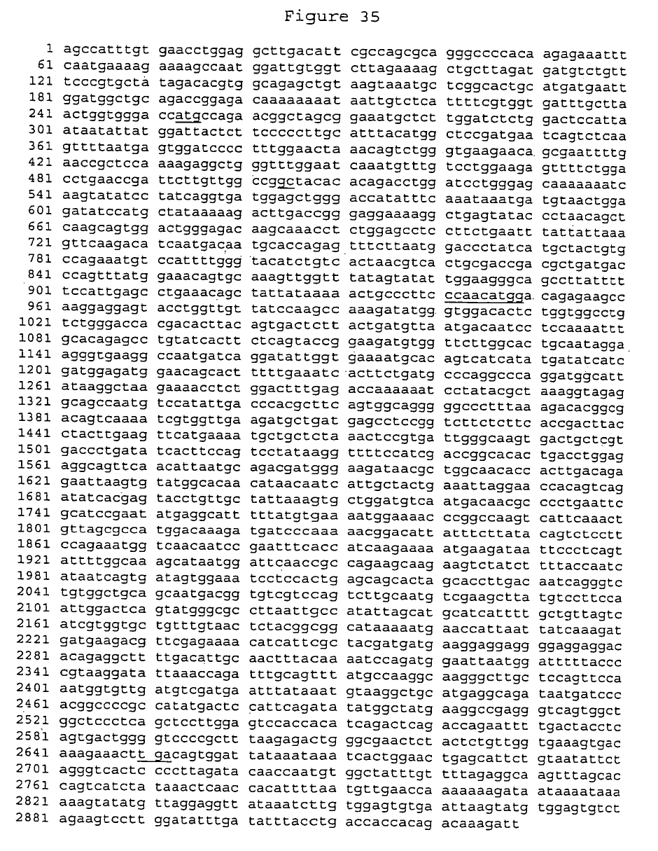

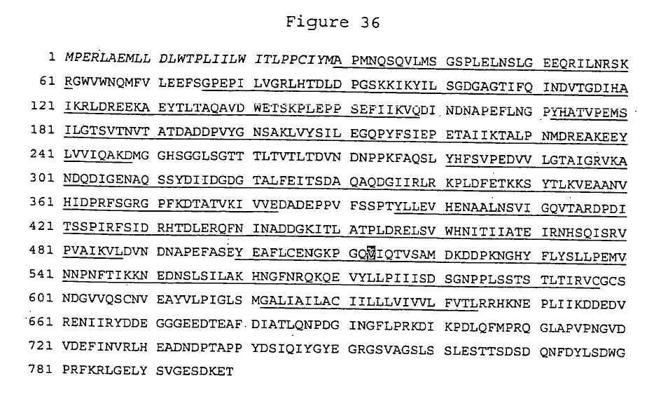

Thus, references herein to the CDH8 gene are understood to relate to the nucleic sequences in the public databases and identified in FIG. 35 and references to the CDH8 protein sequences are understood to relate to the sequences in the public databases and identified in FIG. 36.

In certain jurisdictions claims to methods of treatment are permissible and so the skilled reader will appreciate that the/said SEMCAP3, N33, GRIK4, NPAS3, PDE4B and/or CDH8 gene(s), or fragment(s), derivative(s) or homologue(s) thereof; or SEMCAP3, N33, GRIK4, NPAS3, PDE4B and/or CDH8 protein, or functionally active fragment(s), derivative(s), or homologue(s) thereof, may be administered to an individual as a method of treating an individual with schizophrenia and/or affective psychosis.

“Polynucleotide fragment” as used herein refers to a chain of nucleotides such as deoxyribose nucleic acid (DNA) and transcription products thereof, such as RNA. Naturally, the skilled addressee will appreciate the whole naturally occurring human genome is not included in the definition of polynucleotide fragment.

The polynucleotide fragment can be isolated in the sense that it is substantially free of biological material with which the whole genome is normally associated in vivo. The isolated polynucleotide fragment may be cloned to provide a recombinant molecule comprising the polynucleotide fragment. Thus, “polynucleotide fragment includes double and single stranded DNA, RNA and polynucleotide sequences derived therefrom, for example, subsequences of said fragment and which are of any desirable length. Where a nucleic acid is single stranded then both a given strand and a sequence or reverse complementary thereto is within the scope of the present invention.

In general, the term “expression product” or “gene product” refers to both transcription and translation products of said polynucleotide fragments. When the expression or gene product is a “polypeptide” (i.e. a chain or sequence of amino acids displaying a biological activity substantially similar (eg. 98%, 95%, 90%, 80%, 75% activity) to the biological activity of the protein), it does not refer to a specific length of the product as such. Thus, the skilled addressee will appreciate that “polypeptide” encompasses inter alia peptides, polypeptides and proteins. The polypeptide if required, can be modified in vivo and in vitro, for example by glycosylation, amidation, carboxylation, phosphorylation and/or post-translational cleavage.

The present invention further provides a recombinant or synthetic polypeptide for the manufacture of reagents for use as therapeutic agents in the treatment of schizophrenia and/or affective psychosis. In particular, the invention provides pharmaceutical compositions comprising the recombinant or synthetic polypeptide together with a pharmaceutically acceptable carrier therefor.

The present invention further provides an isolated polynucleotide fragment capable of specifically hybridising to a related polynucleotide sequence from another species. In this manner, the present invention provides probes and/or primers for use in ex vivo and/or in situ detection and expression studies. Typical detection studies include polymerase chain reaction (PCR) studies, hybridisation studies, or sequencing studies. In principle any specific polynucleotide sequence fragment from the identified sequences may be used in detection and/or expression studies. The skilled addressee understands that a specific fragment is a fragment of the sequence which is of sufficient length, generally greater than 10, 12, 14, 16 or 20 nucleotides in length, to bind specifically to the sequence, under conditions of high stringency, as defined herein, and not bind to unrelated sequences, that is sequences from elsewhere in the genome of the organism other than an allelic form of the sequence or non-homologous sequences from other organisms.

“Capable of specifically hybridising” is taken to mean that said polynucleotide fragment preferably hybridises to a related or similar polynucleotide sequence in preference to unrelated or dissimilar polynucleotide sequences.

The invention includes polynucleotide sequence(s) which are capable of specifically hybridising to an polynucleotide fragment as described herein or to a part thereof without necessarily being completely complementary or reverse complementary to said related polynucleotide sequence or fragment thereof. For example, there may be at least 50%, or at least 75%, at least 90%, or at least 95% complementarity. Of course, in some cases the sequences may be exactly reverse complementary (100% reverse complementary) or nearly so (e.g. there may be less than 10, typically less than 5 mismatches). Thus, the present invention also provides anti-sense or complementary nucleotide sequence(s) which is/are capable of specifically hybridising to the disclosed polynucleotide sequence. If a specific polynucleotide is to be used as a primer in PCR and/or sequencing studies, the polynucleotide must be capable of hybridising to related nucleic acid and capable of initiating chain extension from 3′ end of the polynucleotide, but not able to correctly initiate chain extension from unrelated sequences.

If a polynucleotide sequence of the present invention is to be used in hybridisation studies to obtain or identify a related sequence from another organism the polynucleotide sequence should preferably remain hybridised to a sample polynucleotide under stringent conditions. If desired, either the test or sample polynucleotide may be immobilised. Generally the test polynucleotide sequence is at least 10, 14, 20 or at least 50 bases in length. It may be labelled by suitable techniques known in the art. Preferably the test polynucleotide sequence is at least 200 bases in length and may even be several kilobases in length. Thus, either a denatured sample or test sequence can be first bound to a support. Hybridization can be effected at a temperature of between 50 and 70° C. in double strength SSC (2×NaCl 17.5 g/l and sodium citrate (SC) at 8.8 g/l) buffered saline containing 0.1% sodium dodecyl sulphate (SDS). This can be followed by rinsing of the support at the same temperature but with a buffer having a reduced SSC concentration. Depending upon the degree of stringency required, and thus the degree of similarity of the sequences, such reduced concentration buffers are typically single strength SSC containing 0.1% SDS, half strength SSC containing 0.1%SDS and one tenth strength SSC containing 0.1% SDS. Sequences having the greatest degree of similarity are those the hybridisation of which is least affected by washing in buffers of reduced concentration. It is most preferred that the sample and inventive sequences are so similar that the hybridisation between them is substantially unaffected by washing or incubation in standard sodium citrate (0.1×SSC) buffer containing 0.1% SDS.

Oligonucleotides may be designed to specifically hybridise to N33 SEMCAP3, NPAS3, GRIK4, PDE4B and/or CDH8 nucleic acid. They may be synthesised, by known techniques and used as primers in PCR or sequencing reactions or as probes in hybridisations designed to detect the presence of a mutated or normal N33, SEMCAP3, NPAS3, GRIK4, PDE4B and/or CDH8 gene(s) in a sample. The oligonucleotides may be labelled by suitable labels known in the art, such as, radioactive labels, chemiluminescent labels or fluorescent labels and the like.

The term “oligonucleotidell is not meant to indicate any particular length of sequence and encompasses nucleotides of preferably at least 10b (e.g. 10b to 1 kb) in length, more preferably 12b-500b in length and most preferably 15b to 100b.

The oligonucleotides may be designed with respect to any of the sequences described herein and may be manufactured according to known techniques. They may have substantial sequence identity (e.g. at least 50%, at least 75%, at least 90% or at least 95% sequence identity) with one of the strands shown therein or an RNA equivalent, or with a part of such a strand. Preferably such a part is at least 10, at least 30, at least 50 or at least 200 bases long. It may be an open reading frame (ORF) or a part thereof.

Oligonucleotides which are generally greater than 30 bases in length should preferably remain hybridised to a sample polynucleotide under one or more of the stringent conditions mentioned above. Oligonucleotides which are generally less than 30 bases in length should also preferably remain hybridised to a sample polynucleotide but under different conditions of high stringency. Typically the melting temperature of an oligonucleotide less than 30 bases may be calculated according to the formula of; 2° C. for every A or T, plus 4° C. for every G or C, minus 5° C. Hybridization may take place at or around the calculated melting temperature for any particular oligonucleotide, in 6×SSC and 1% SDS. Non specifically hybridised oligonucleotides may then be removed by stringent washing, for example in 3×SSC and 0.1% SDS at the same temperature only substantially similar matched sequences remain hybridised i.e. said oligonucleotide and corresponding test nucleic acid.

When oligonucleotides of generally less than 30 bases in length are used in sequencing and/or PCR studies, the melting temperature may be calculated in the same manner as described above. The oligonucleotide may then be allowed to anneal or hybridise at a temperature around the oligonucleotides calculated melting temperature. In the case of PCR studies the annealing temperature should be around the lower of the calculated melting temperatures for the two priming oligonucleotides. It is to be appreciated that the conditions and melting temperature calculations are provided by way of example only and are not intended to be limiting. It is possible through the experience of the experimenter to vary the conditions of hybridisation and thus anneal/hybridise oligonucleotides at temperatures above their calculated melting temperature. Indeed this can be desirable in preventing so-called non-specific hybridisation from occurring.

It is possible when conducting PCR studies to predict an expected size or sizes of PCR product(s) obtainable using an appropriate combination of two or more oligonucleotides, based on where they would hybridise to the sequences described herein. If, on conducting such a PCR on a sample of DNA, a fragment of the predicted size is obtained, then this is predictive that the DNA encodes a homologous sequence from a test organism.

Proteins for all the applications described herein can be produced by cloning the gene for example into plasmid vectors that allow high expression in a system of choice e.g. insect cell culture, yeast, animal cells, bacteria such as Escherichia coli. To enable effective purification of the protein, a vector may be used that incorporates an epitope tag (or other “sticky” extension such as His6) onto the protein on synthesis. A number of such vectors and purification systems are commercially available.

The polynucleotide fragment can be molecularly cloned into a prokaryotic or eukaryotic expression vector using standard techniques and administered to a host. The expression vector is taken up by cells and the polynucleotide fragment of interest expressed, producing protein.

It will be understood that for the particular polypeptides embraced herein, natural variations such as may occur due to polymorphisms, can exist between individuals or between members of the family. These variations may be demonstrated by (an) amino acid difference(s) in the overall sequence or by deletions, substitutions, insertions, inversions or additions of (an) amino acid(s) in said sequence. All such derivatives showing the recognised activity are included within the scope of the invention. For example, for the purpose of the present invention conservative replacements may be made between amino acids within the following groups:

(I) Alanine, serine, threonine;

(

(II) Glutamic acid and aspartic acid;

(III) Arginine and leucine;

(IV) Asparagine and glutamine;

(V) Isoleucine, leucine and valine;

(VI) Phenylalanine, tyrosine and tryptophan

Moreover, recombinant DNA technology may be used to prepare nucleic acid sequences encoding the various derivatives outlined above.

As is well known in the art, the degeneracy of the genetic code permits substitution of bases in a codon resulting in a different codon which is still capable of coding for the same amino acid, e.g. the codon for amino acid glutamic acid is both GAT and GAA. Consequently, it is clear that for the expression of polypeptides from nucleotide sequences described herein or fragments thereof, use can be made of a derivative nucleic acid sequence with such an alternative codon composition different from the nucleic acid sequences shown in the Figures.

The polynucleotide fragments of the present invention are preferably linked to regulatory control sequences. Such control sequences may comprise promoters, operators, inducers, enhancers, silencers, ribosome binding sites, terminators etc. Suitable control sequences for a given host may be selected by those of ordinary skill in the art.

A polynucleotide fragment according to the present invention can be ligated to various expression controlling sequences, resulting in a so called recombinant nucleic acid molecule. Thus, the present invention also-includes an expression vector containing an expressible nucleic acid molecule. The recombinant nucleic acid molecule can then be used for the transformation of a suitable host.

Specific vectors which can be used to clone nucleic acid sequences according to the invention are known in the art (e.g. Rodriguez, R. L. and Denhadt, D. T., Edit., Vectors: a survey of molecular cloning vectors and their uses, Butterworths, 1988, or Jones et al., Vectors: Cloning Applications: Essential Techniques (Essential techniques series), John Wiley & Son. 1998).

The methods to be used for the construction of a recombinant nucleic acid molecule according to the invention are known to those of ordinary skill in the art and are inter alia set forth in Sambrook, et al. (Molecular Cloning: a laboratory manual Cold Spring Harbour Laboratory, 1989).

The present invention also relates to a transformed cell containing the polynucleotide fragment in an expressible form. “Transformation”, as used herein, refers to the introduction of a heterologous polynucleotide fragment into a host cell. The method used may be any known in the art, for example, direcr uptake, transfection transduction or electroporation (Current Protocols in Molecular Biology, 1995. John Wiley and Sons Inc.). The heterologous polynucleotide fragment may be maintained through autonomous replication or alternatively, may be integrated into the host genome. The recombinant nucleic acid molecules preferably are provided with appropriate control sequences compatible with the designated host which can regulate the expression of the inserted polynucleotide fragment, e.g. tetracycline responsive promoter, thymidine kinase promoter, SV-40 promoter and the like.

Suitable hosts for the expression of recombinant nucleic acid molecules may be prokaryotic or eukaryotic in origin. Hosts suitable for the expression of recombinant nucleic acid molecules may be selected from bacteria, yeast, insect cells and mammalian cells.

In another aspect the present invention also relates to a method of diagnosing schizophrenia and/or affective psychosis or susceptibility to schizophrenia and/or affective psychosis in an individual, wherein the method comprises determining if SEMCAP3, N33, GRIK4, NPAS3, PDE4B and/or CDH8 gene(s) in the individual has been disrupted by a mutation or chromosomal rearrangement.

The methods which may be employed to elucidate such a mutation or chromosomal rearrangement are well known to those of skill in the art and could be detected for example using PCR or in hybridisation studies using suitable probes which could be designed to span an identified mutation site or chromosomal breakpoint in close proximity to the/said N33 SEMCAP3, NPAS3, GRIK4, PDE3B and/or CDH8 gene(s), such as the breakpoint identified by the present inventors and described herein.

Once a particular polymorphism or mutation -has been identified it may be possible to determine a particular course of treatment. For example it is known that some forms of treatment work for some patients, but not all. This may in fact be due to mutations in the/said N33, SEMCAP3, NPAS3, GRIK4, PDE4B and/or CDH8 gene(s) or surrounding sequence, and it may therefore be possible to determine a treatment strategy using current therapies, based on a patient's genotype.

It will be appreciated that mutations in the gene sequence or controlling elements of a gene, eg. a promoter and/or enhance can have subtle effects such as affecting mRNA splicing/stability/activity and/or control of gene expression levels, which can also be determined. Also the relative levels of RNA can be determined using for example hybridisation or quantitative PCR as a means to determine if the/said SEMCAP3, N33, GRIK4, NPAS3, PDE4B and/or CHD8 gene(s) has been disrupted.

Moreover the presence and/or levels of the/said SEMCAP3, N33, GRIK4, NPAS3, PDE4B and/or CHD8 gene(s) products themselves can be assayed by immunological techniques such as radioimmunoassay, Western blotting and ELISA using specific antibodies raised against the gene products. The present invention also therefore relates to antibodies specific for a SEMCAP3, N33, GRIK4, NPAS3, PDE4B and/or CHD8 gene(s) product(s) and uses thereof in diagnosis and/or therapy.

A further aspect of the present invention therefore provides antibodies specific to the polypeptides of the present invention or epitopes thereof. Production and purification of antibodies specific to an antigen is a matter of ordinary skill, and the methods to be used are clear to those skilled in the art. The term antibodies can include, but is not limited to polyclonal antibodies, monoclonal antibodies (mAbs), humanised or chimeric antibodies, single chain antibodies, Fab fragments, F(ab′)₂ fragments, fragments produced by a Fab expression library, anti-idiotypic (anti-Id) antibodies, and epitope binding fragments of any of the above. Such antibodies may be used in modulating the expression or activity of the particular polypeptide, or in detecting said polypeptide in vivo or in vitro.

Using the sequences disclosed herein, it is possible to identify related sequences in other animals, such as mammals, with the intention of providing an animal model for psychiatric disorders associated with the improper functioning of the nucleotide sequences and proteins of the present invention. Once identified, the homologous sequences can be manipulated in several ways common to the skilled person in order to alter the functionality of the nucleotide sequences and proteins homologous to those of the present invention. For example, “knock-out” animals may be created, that is, the expression of the genes comprising the nucleotide sequences homologous to those of the present invention may be reduced or substantially eliminated in order to determine the effects of reducing or substantially eliminating the expression of such genes. Alternatively, animals may be created where the expression of the nucleotide sequences and proteins homologous to those of the present invention are upregulated, that is, the expression of the genes comprising the nucleotide sequences homologous to those of the present invention may be increased in order to determine the effects of increasing the expression of these genes. In addition to these manipulations substitutions, deletions and additions may be made to the nucleotide sequences encoding the proteins homologous to those of the present invention in order to effect changes in the activity of the proteins to help elucidate the function of domains, amino acids, etc. in the proteins. Furthermore, the sequences of the present invention may also be used to transform animals to the manner described above. The manipulations described above may also be used to create an animal model of schizophrenia and/or affective psychosis associated with the improper functioning of the nucleotide sequences and/or proteins of the present invention in order to evaluate potential agents which may be effective for combatting psychotic disorders, such as schizophrenia and/or affective psychosis.

Thus, the present invention also provides for screens for identifying agents suitable for preventing and/or treating schizophrenia and/or affective psychosis associated with disruption or alteration in the expression of the SEMCAP3, N33, GRIK4, NPAS3, PDE3B and/or CHD8 gene and/or its gene products. Such screens may easily be adapted to be used for the high throughput screening of libraries of compounds such as synthetic, natural or combinatorial compound libraries.

Thus, the/said SEMCAP3, N33, GRIK4, NPAS3, PDE4B and/or CDH8 gene(s) products according to the present invention can be used for the in vivo or in vitro identification of novel ligands or analogs thereof. For this purpose binding studies can be performed with cells transformed with nucleotide fragments according to the invention or an expression vector comprising a polynucleotide fragment according to the invention, said cells expressing the/said SEMCAP3, N33, GRIK4, NPAS3, PDE4B and/or CDH8 gene(s) products according to the invention.

Alternatively also the/said SEMCAP3, N33, GRIK4, NPAS3, PDE4B and/or CDH8 gene(s) products according to the invention as well as ligand-binding domains thereof can be used in an assay for the identification of functional liqands or analogs for the/said SEMCAP3, N33, GRIK4, NPAS3, PDE4B and/or CDH8 gene(s) products.

Methods to determine binding to expressed gene products as well as in vitro and in vivo assays to determine biological activity of gene products are well known. In general, expressed gene product is contacted with the compound to be tested and binding, stimulation or inhibition of a functional response is measured.

Thus, the present invention provides for a method for identifying ligands for SEMCAP3, N33, GRIK4, NPAS3, PDE4B and/or CDH8 gene(s) products, said method comprising the steps of:

a) introducing into a suitable host cell a polynucleotide fragment according to the invention;

b) culturing cells under conditions to allow expression of the polynucleotide fragment;

c) optionally isolating the expression product;

d) bringing the expression product (or the host cell from step b)) into contact with potential ligands which will possibly bind to the protein encoded by said polynucleotide fragment from step a);

e) establishing whether a ligand has bound to the expressed protein; and

f) optionally isolating and identifying the ligand.

As a preferred way of detecting the binding of the ligand to the expressed protein, also signal transduction capacity may be measured.

Compounds which activate or inhibit the function of SEMCAP3, N33, GRIK4, NPAS3, PDE4B and/or CDH8 gene(s) products may be employed in therapeutic treatments to activate or inhibit the polypeptides of the present invention.

The present invention will now be further described by way of Example and with reference to the Figures which show:

FIG. 1 shows an ideogram diagram of the chromosomal rearrangement (a reciprocal translocation) in patient 1. The two breakpoints are marked at the approximate chromosomal locations at which they are located. In addition, and not to scale, the two candidate disease-causing genes, N33 and SEMCAP3, are placed in the correct orientation and with respect to the breakpoints.

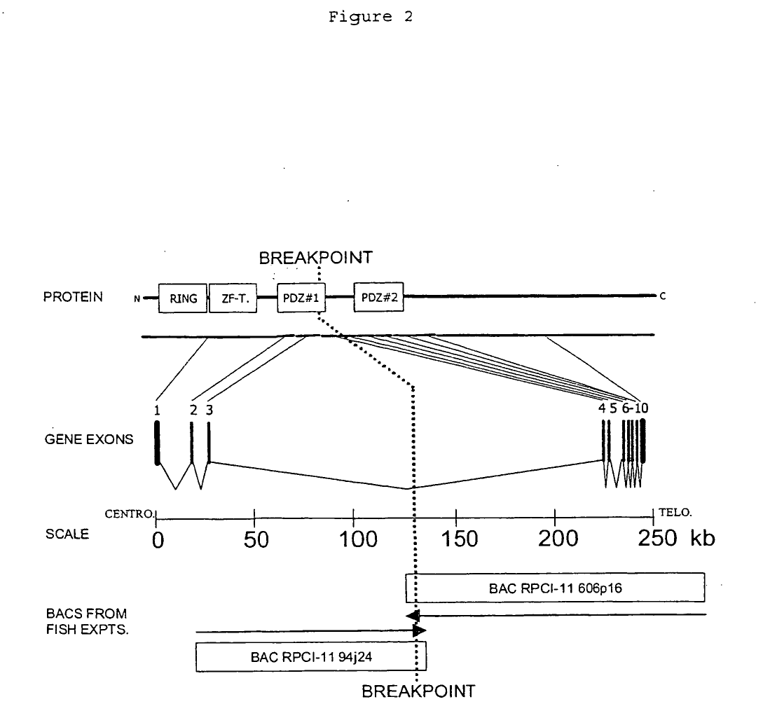

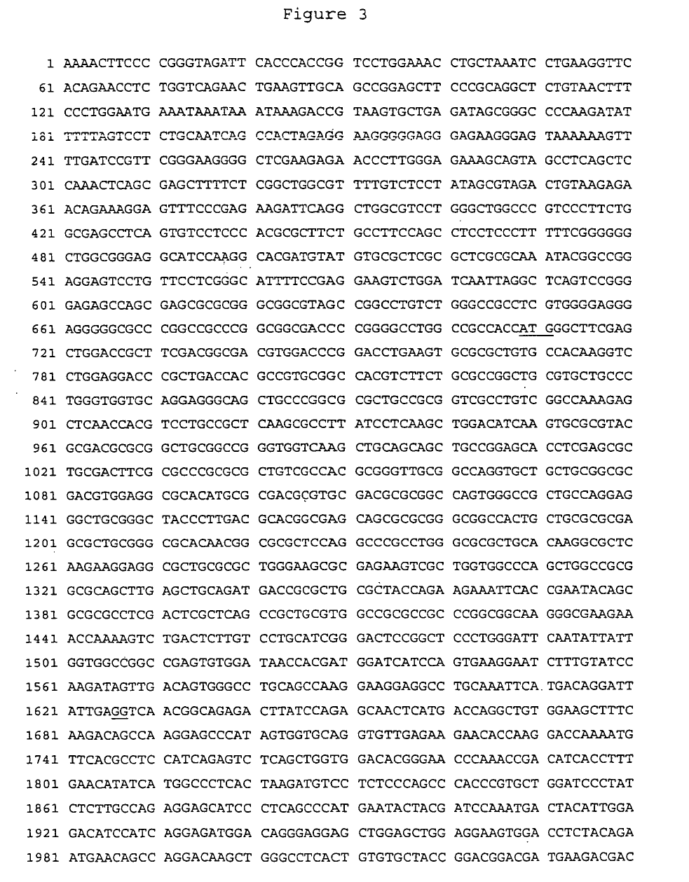

FIG. 2 shows a representation of the genomic structure of the SEMCAP3 gene: its spliced exons spread over a genomic extent of approximately 250 kb. Above the gene, the coding contribution of each exon to the SEMCAP3 protein is indicated by bars and finely dashed lines. The domain structure of SEMCAP3 protein is shown at the top of the figure. ‘RING’ refers to a RING-finger domain, ‘ZF-T.’ to a TRAF-type zinc finger (also referred to as a sina domain) and ‘PDZ’ to PDZ domain present in PSD-95, Dlg, and ZO-1/2. The BAC clones used to identify the breakpoint location are included at the bottom of the figure together with the inferred direction (arrows) of the breakpoint from the FISH results using these clones. The heavy dashed line shows the position of the breakpoint with respect to the gene exons and the domain structure of the protein.

FIG. 3 Nucleic acid sequence of Human SEMCAP3 (genomic DNA sequence including CpG island/putative promoter upstream of 5′ UTR/cDNA sequence is also included for clarity). The following features are marked for clarity:
a) ATG start site located at position 709 (underlined)
b) GG bases (underlined) at the junction between exons 3 and 4 (i.e. between which the breakpoint is located)
c) UAA stop codon located at position 3907 (underlined).

FIG. 4 Amino acid sequence of Human SEMCAP3 with underlined regions of interest.
a) Residues 18-55 Ring finger domain
b) Residues 101-158 SINA/ZF-TRAF domain
c) Residues 246-339 PDZ domain #1
d) Residues 418-504 PDZ domain #2

FIG. 5 shows a schematic representation of the N33 gene: exon splicing and chromosome breakpoint identified in the present invention.

FIG. 6 shows the nucleotide sequence of the various exons for N33.

FIG. 7 shows the various transcript options and associated amino acid sequences of the transcripts for N33;

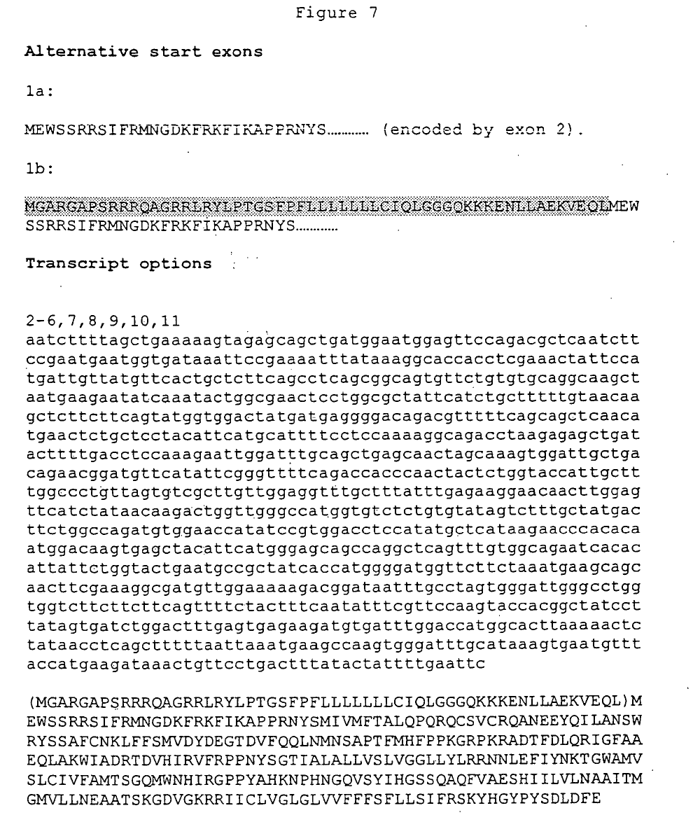

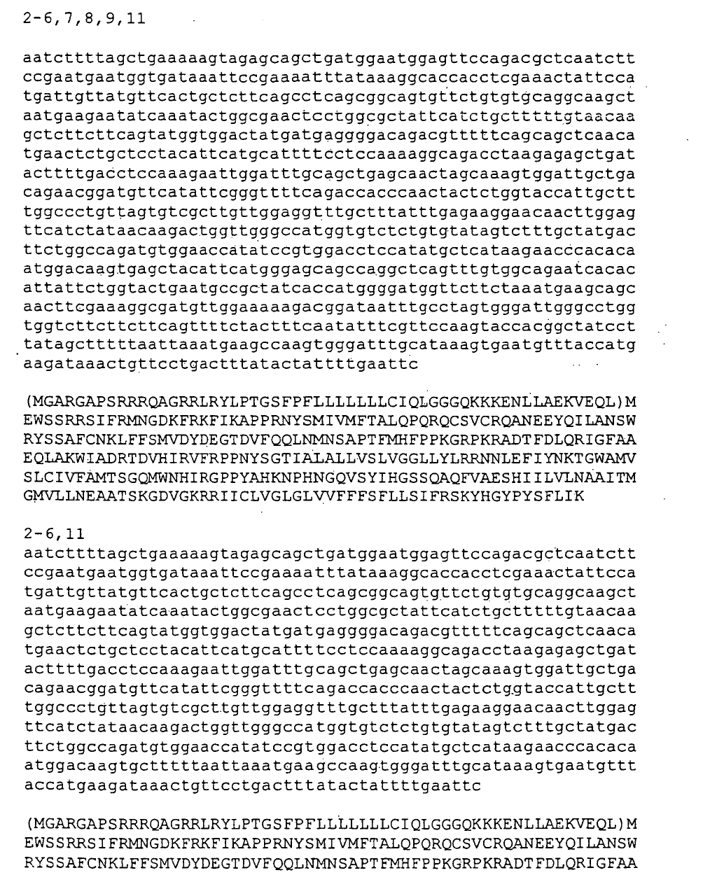

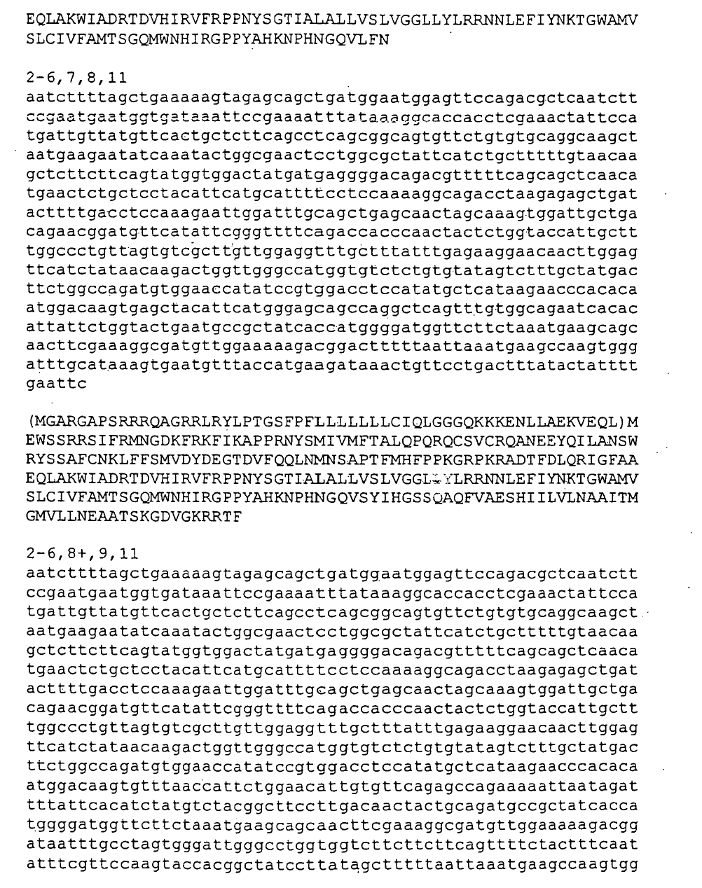

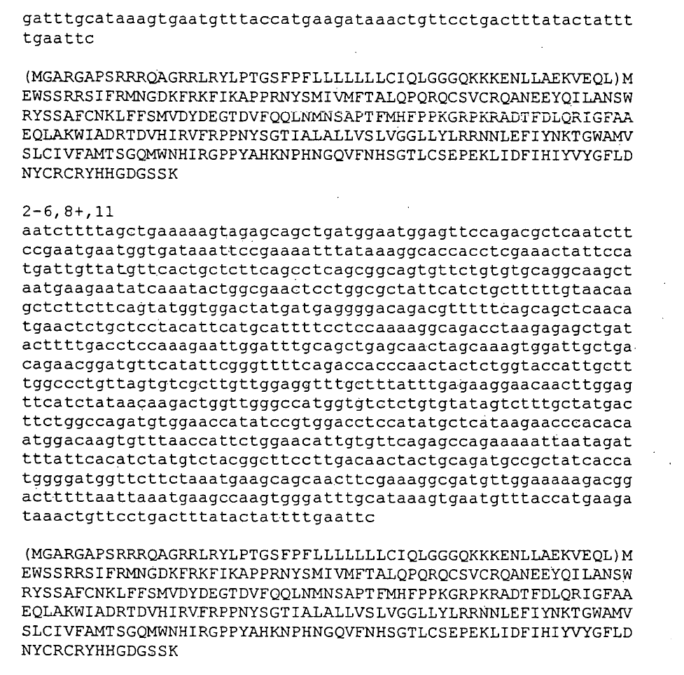

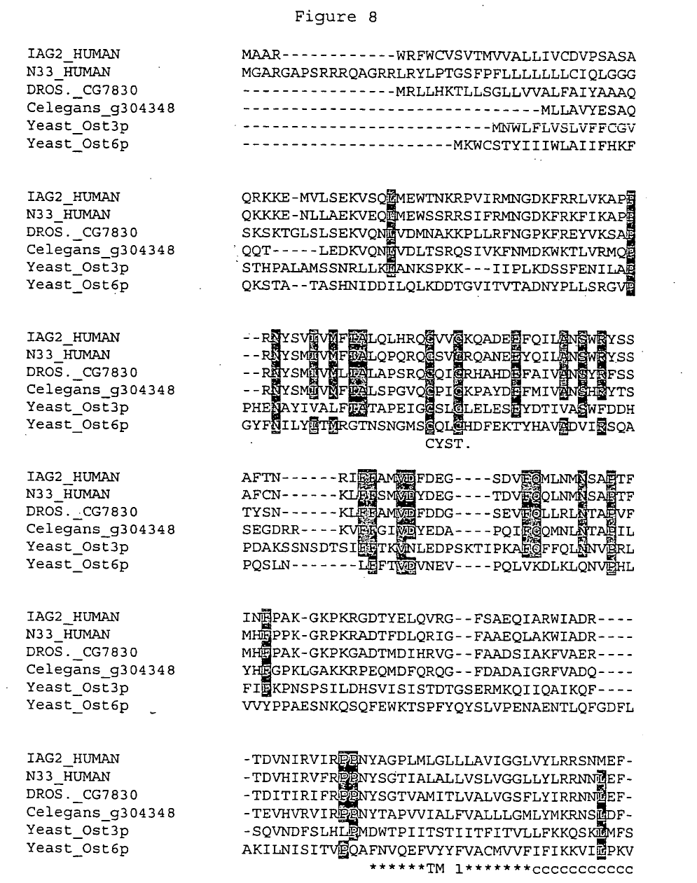

FIG. 8 shows N33 protein aligned with other homologues.

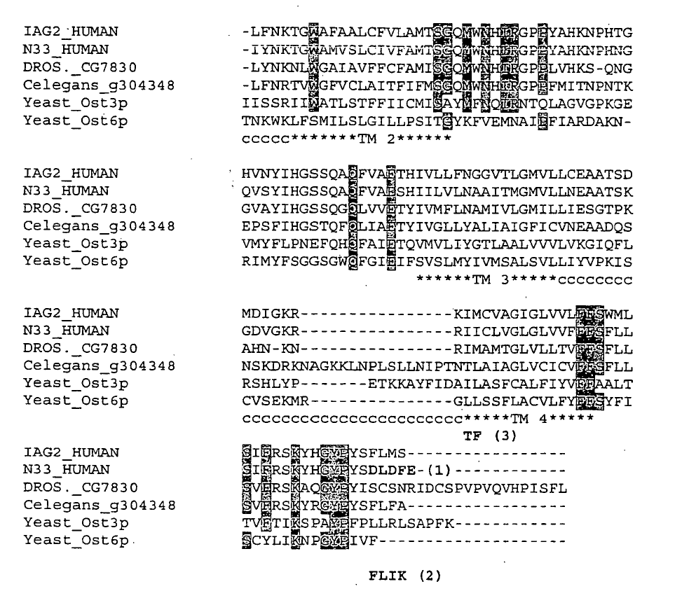

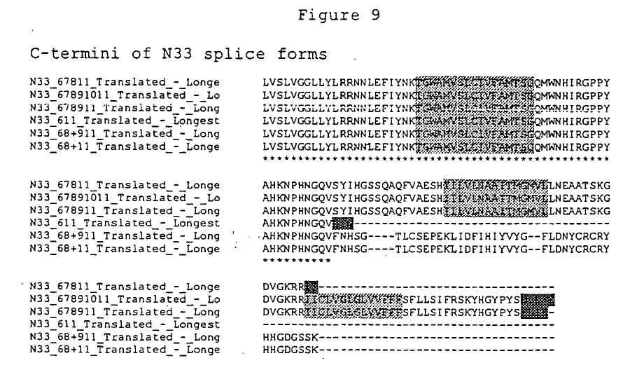

FIG. 9 shows the effect of the C-terminus of the various N33 splice forms. The variety of splice forms at the 3′ end of the gene has implications for the C-terminus of the protein. This is especially important when it is considered that N33 is likely to reside in the Golgi/ER compartment of the cell where C-termini are often involved in anchoring or trafficking proteins to different organelles. The light grey shading indicates putative transmembrane domains. Hence, only the spliceforms with exons 1a/1b, 2-6, 7, 8, 9, 10, 11 or 1a/1b, 2-6, 7, 8, 9, 11 are likely to encode functional proteins and these will only differ in the extreme C-terminal residues.

FIG. 10 shows the published nucleotide sequence for GRIK4.

FIG. 11 shows the published amino acid sequence for GRIK4.

FIG. 12 Breakpoints identified in the subject (patient 2). CEPH library YACs (Chumakov et al, 1992) spanning the breakpoints are listed. Also detailed are the BAC clones (and accession numbers) from the RPCI-11 BAC library (Osoegawa et al, 2001) that span or flank (indicated by dashes) the breakpoints. Breakpoints at 8q13 were not characterised in this study.

FIG. 13 Representation of complex chromosomal rearrangement in the subject (patient 2). The pericentric chromosome 2 inversion is coupled with a translocation to chromosome 11. The chromosome 11 region between the 11q23.3 and 11q24.3 breakpoints is inserted on chromosome 8q13.

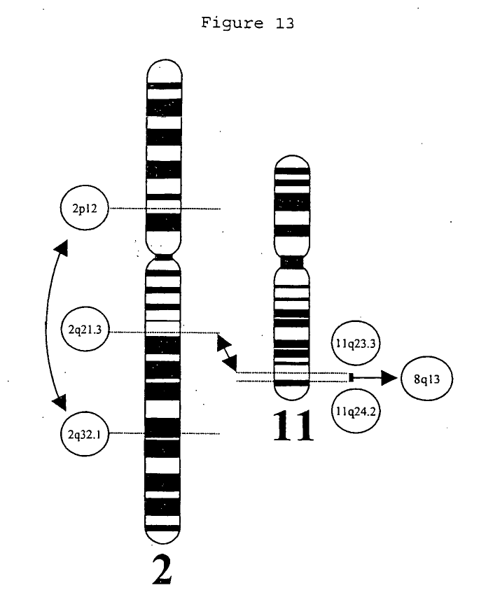

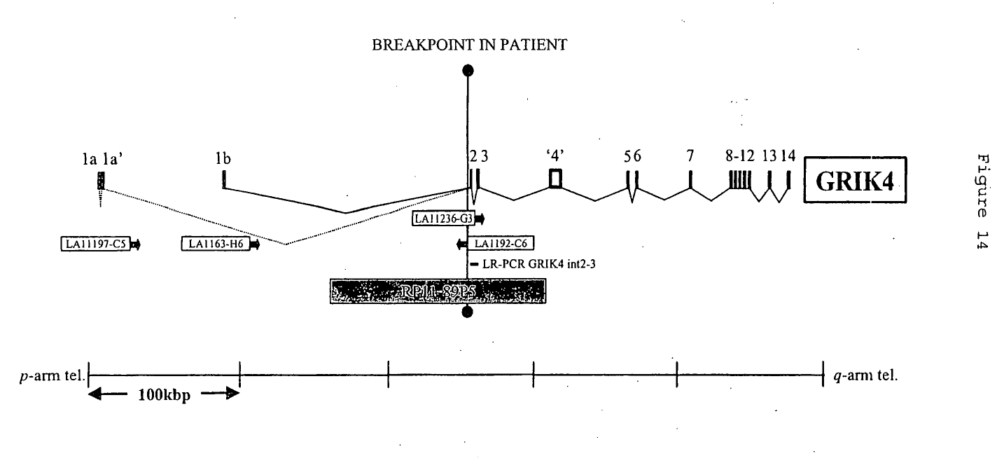

FIG. 14 Genomic arrangements of the GRIK4 gene disrupted in the subject. Two potential GRIK4 transcripts with alternative start-sites are indicated. The 1a/1a′ exons are derived from EST BE388730. The transcript containing the 1b exon corresponds to the published GRIK4 sequence (acc. S67803). It is probable that the present inventors exon “4” corresponds to a number of undefined exons which can only be subdivided after release of genomic sequence over this part of the gene. Hence, the actual number of GRIK4 transcript exons will most likely exceed 14. BAC (grey boxes), cosmid (white boxes) and long-range PCR product (black line) derived FISH probes enabled the positioning of the breakpoint (arrows indicate the relative direction of the breakpoint deduced from the presence/absence of the signals on the two derived chromosomes). Probes from BAC RPCI-11 89P5 and cosmids LA11197-C5, LA1163-H6, LA11236-G3 and LA1192-C6 indicated that the breakpoint was located near exons 2 and 3. A FISH probe synthesized from a long-range PCR product corresponding to the intronic sequence between these two exons indicated that the breakpoint lies upstream of the intron between exons 2 and 3.

FIG. 15 5′ sequence of the GRIK4 gene showing the two possible N-terminal peptides derived from alternate start sites. Exon combination 1a-1a′-2 is derived from an EST sequence (acc. BE388730). Exon combination 1b-2 is based on the published cDNA sequence (e.g. acc. S67803). The actual amino acid sequence may differ from the published amino acid sequence as there is a potential downstream methionine start (MVAC . . . instead of MPRV . . . ) containing a more conserved Kozak sequence (Kozak, 1986). It can be seen that the breakpoint upstream of exon 2 will separate the majority of the coding sequence from the promoter resulting in a putative null allele. Exonic DNA sequence is shown in capitals, intronic or upstream sequence in lower case. Conserved splice junction sequences (EXON/GT - - - AG/EXON) are underlined. Single letter amino acid codes are shown beneath the appropriate DNA codons. A functional C/G:Leu/Val single nucleotide polymorphism (underlined) is found within exon 2.

FIG. 16 shows the complete alternative nucleic acid sequence as identified by the present inventors.

FIG. 17 shows the complete alternative amino acid sequence as identified by the present inventors.

FIG. 18 shows the nucleic acid sequence of NPAS3 spliceform 1.

FIG. 19 shows the protein sequence of NPAS3 spliceform 1.

FIG. 20 shows the nucleic acid sequence of NPAS3 spliceform 2.

FIG. 21 shows the protein sequence of NPAS3 spliceform 2.

FIG. 22 shows an ideogram representation of the balanced translocation in patient 3 relating to this invention.

FIG. 23 shows the genomic arrangement of the NPAS3 gene including the position of the observed breakpoint.

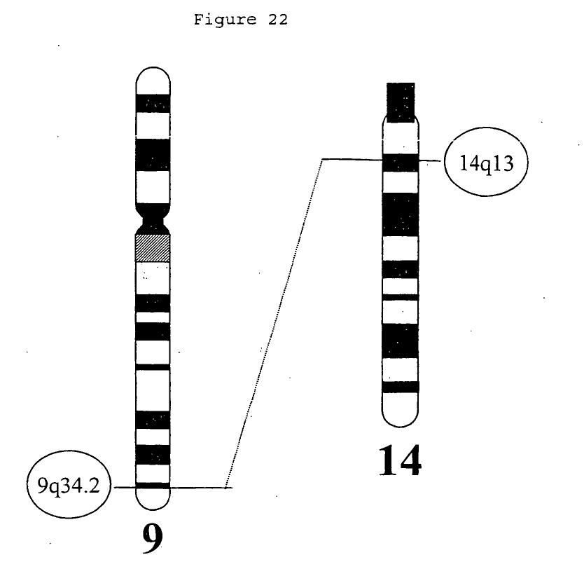

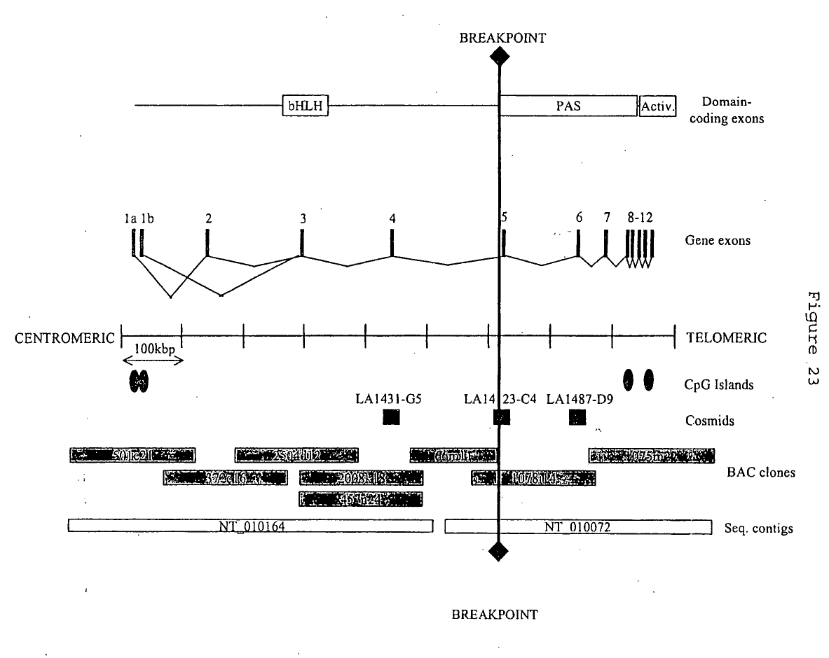

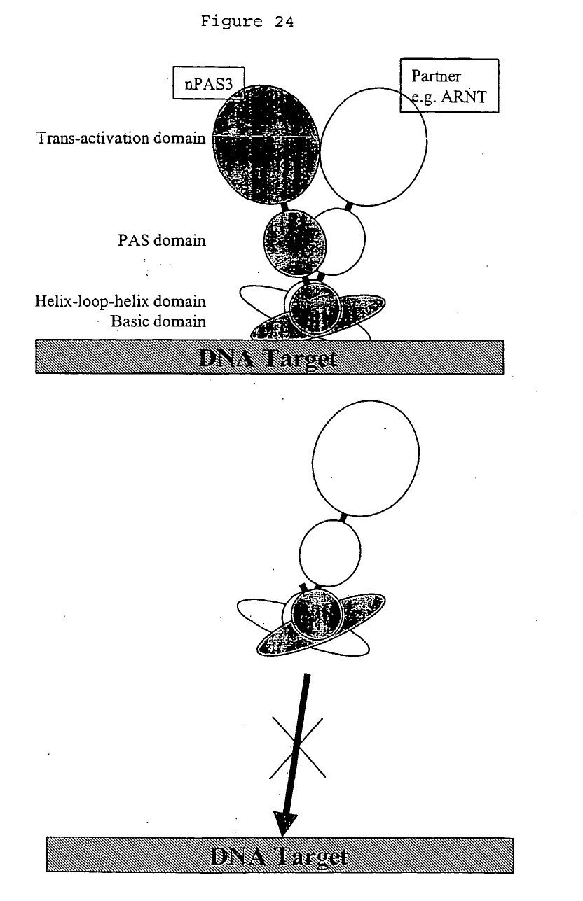

FIG. 24 shows potential functional consequences of the disruption to NPAS3 gene: dominant-negative activity.

FIG. 25 shows the PDE4B1 nucleic acid sequence.

FIG. 26 shows the PDE4B1 protein sequence.

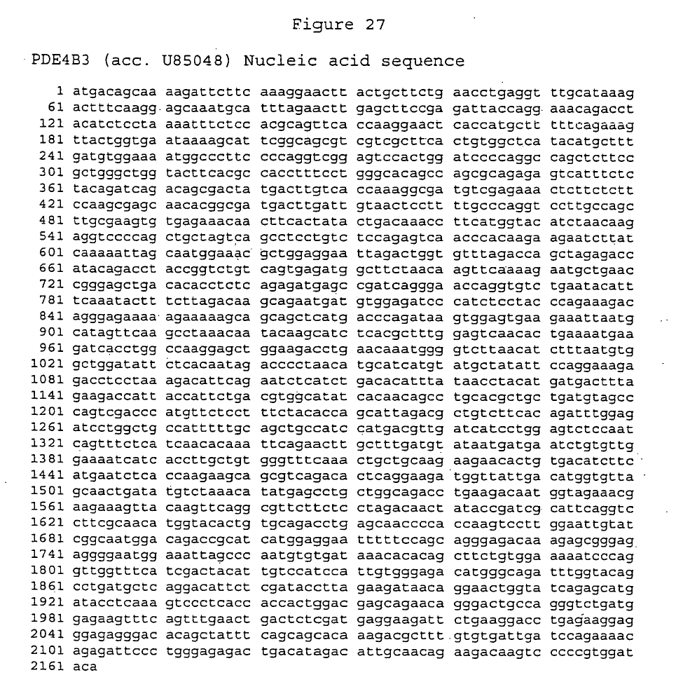

FIG. 27 shows the PDE4B3 nucleic acid sequence.

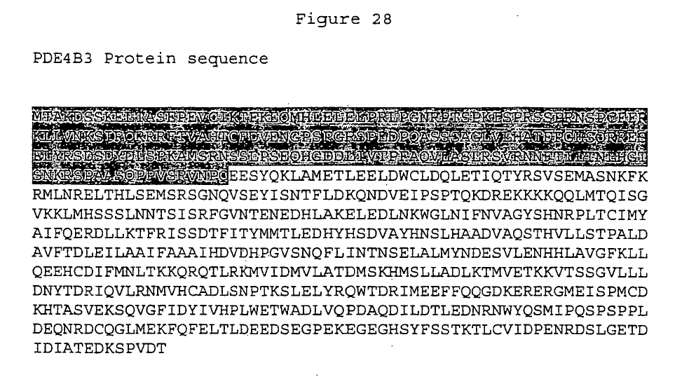

FIG. 28 shows the PDE4B3 protein sequence.

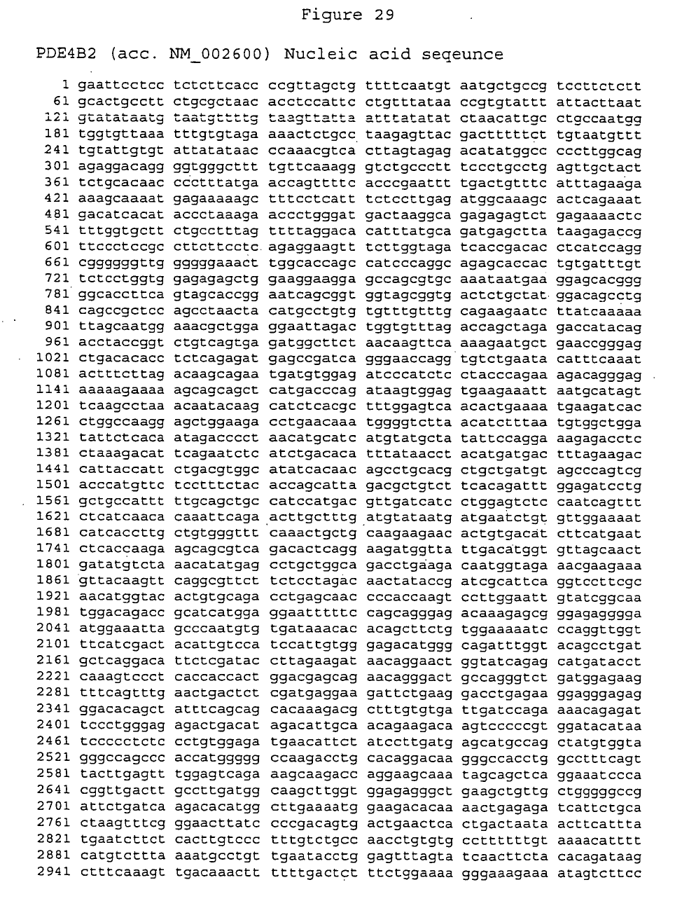

FIG. 29 shows the PDE4B2 nucleic acid sequence.

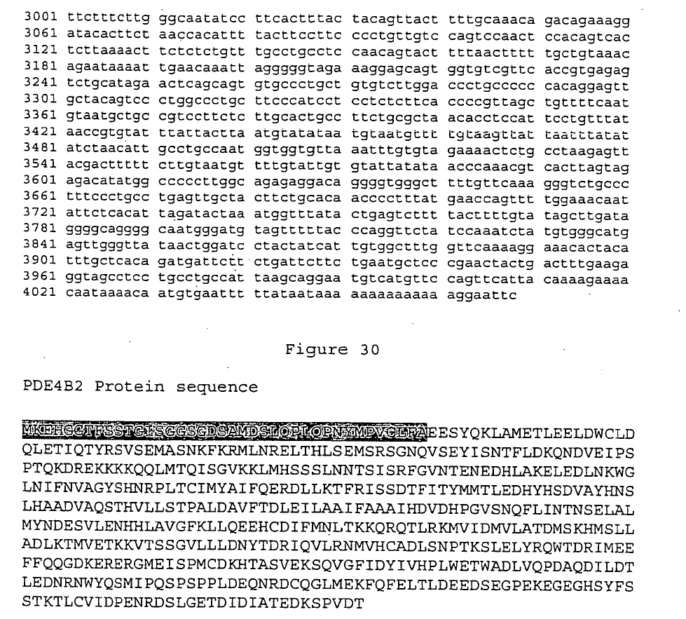

FIG. 30 shows the PDE4B2 protein sequence.

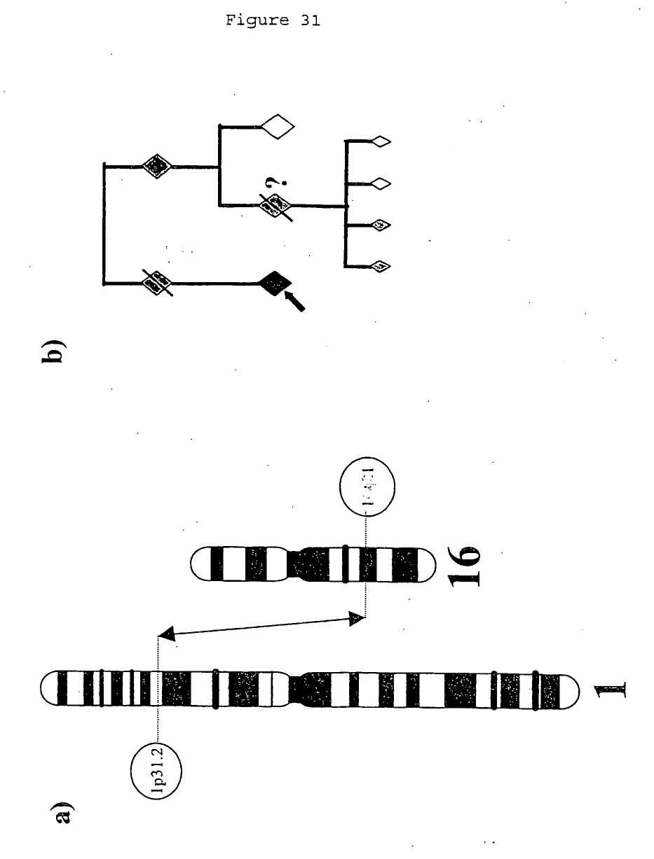

FIG. 31 a) Ideogram representation of balanced translocation between chromosomes 1 and 16 in patient 4.

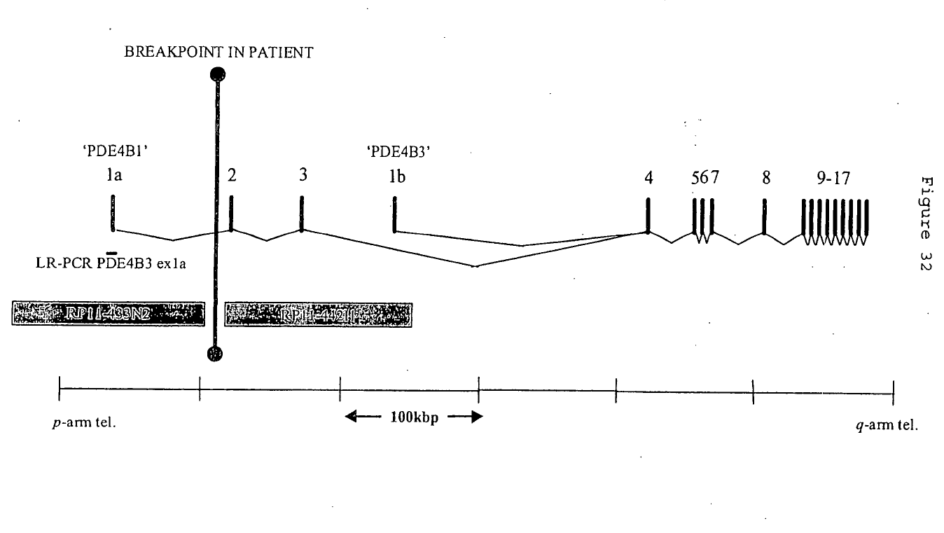

FIG. 32 Genomic arrangements of the PDE4B gene disrupted in the subject (patient 4). The two long transcripts of the PDE4B gene are shown. FISH showed the breakpoint was within a gap in the genome sequence between BACs RPCI-11 433N2 and RPCI-11 442I1. This positioned the breakpoint between the first and second exons of the PDE4B1 form of the gene (acc. L20966). A long-range PCR product FISH probe corresponding to the genomic region encompassing the 1a exons of PDE4B1 confirmed that the gene was disrupted between exon pairs la and exon 2 (i.e. only PDE4B1 transcripts are directly disrupted by the chromosome abnormality).

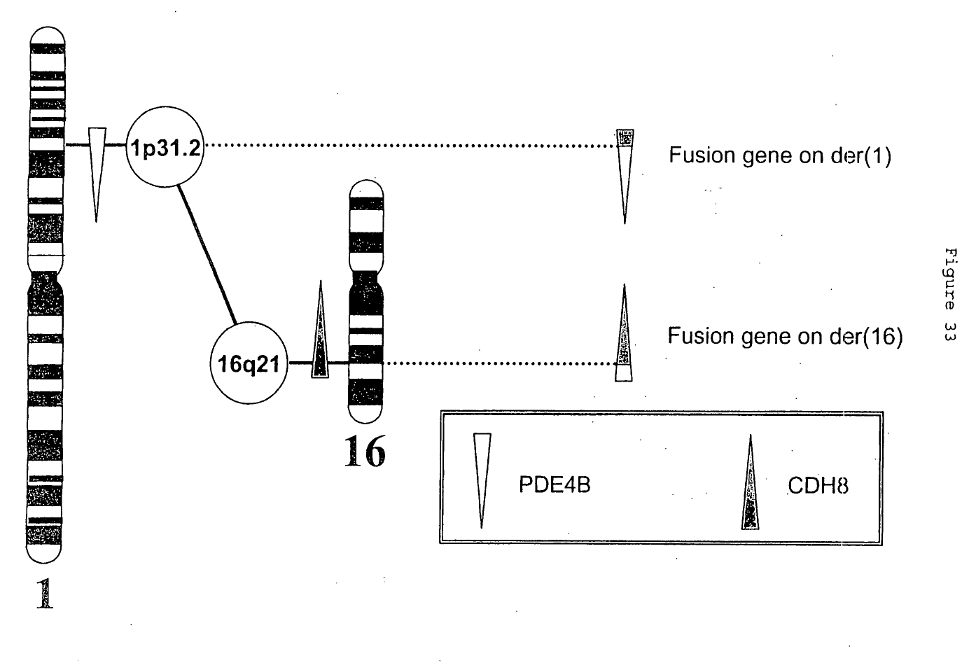

FIG. 33 shows an ideogram diagram of the chromosomal rearrangement (a reciprocal translocation) in patient 4. The two breakpoints are marked at the approximate chromosomal locations at which they are located. In addition, and not to scale, the two candidate disease-causing genes, PDE4B and CDH8, are placed in the correct orientation and with respect to the breakpoints. The fusion genes on derived chromosomes 1 and 16 that result from the reciprocal translocation are also indicated, demonstrating the potential capacity for fusion transcript/protein synthesis.

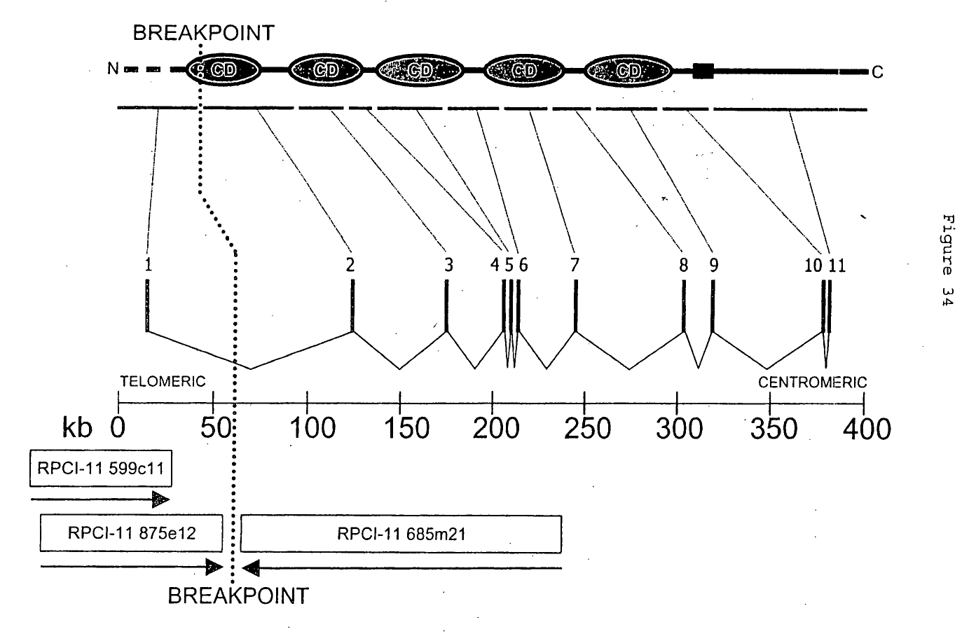

FIG. 34 shows a representation of the genomic structure of the CDH8 gene: its spliced exons spread over a genomic extent of approximately 400 kb.

Above the gene, the coding contribution of each exon to the CDH8 protein is indicated by bars and finely dashed lines. The domain structure of CDH8 protein is shown at the top of the figure. ‘N’ and ‘C’ refer to the N- and C-termini of the protein. The broken line at the N-terminus indicates the existence of signal peptide and proprotein domains—both of which are cleaved off in the mature protein. The ‘CD’ ovals represent the positions of the five extracellular cadherin domains. The black box signifies the position of the hydrophobic stretch of amino acids that act as the membrane-spanning domain. The BAC clones used to identify the breakpoint location are included at the bottom of the figure together with the inferred direction (arrows) of the breakpoint from the FISH results using these clones. The heavy dashed line shows the position of the breakpoint with respect to the gene exons and the domain structure of the protein.

FIG. 35 Nucleic acid sequence of Human CDH8. The following features are marked for clarity:
a) ATG start site located at position 253 (underlined)
b) GC bases (underlined) at the junction between exons 1 and 2 (i.e. between which the breakpoint is located)
c) UGA stop codon located at position 2650 (underlined).

FIG. 36 Amino acid sequence of Human CDH8 with underlined regions of interest.
a) Residues 1-29 signal peptide domain (italics)
b) Residues 30-61 propeptide fragment cleaved off in mature protein.
c) Residues 76-158 cadherin domain #1 (underlined)
d) Residues 172-248 cadherin domain #2 (underlined)
e) Residues 281-383 cadherin domain #3 (underlined)
f) Residues 396-487 cadherin domain #4 (underlined)
g) Residues 500-597. cadherin domain #5 (underlined)
h) ‘V’ highlighted at position 513 is the last residue in common with the putative truncated rat protein product from the alternatively spliced form.
i) Residues 622-645 transmembrane domain #1 (underlined).

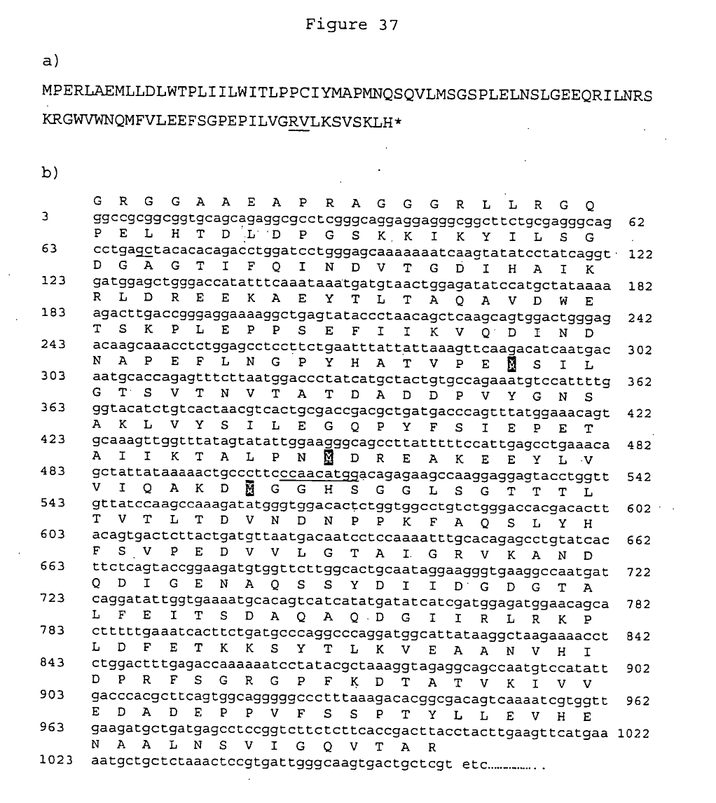

FIG. 37
a) Fusion protein product resulting from CDH8 promoter/exon 1 spliced to PDE4B exon 2 and beyond (transcribed on der(16)). The underlined residues ‘RV’ represent the fusion site between the two genes.
b) Fusion protein product resulting from PDE4B promoter (long form)/exon la spliced to CDH8 exon 2 and beyond (transcribed on der(1)). See text for details: only the reading frame producing the N-terminal truncated form of the CDH8 protein is shown The underlined ‘gc’ at position 68 represents the point of fusion between the two genes. Three potential methionine translation start sites are shown (highlighted) with the second of these having a nucleic acid sequence most similar to the canonical Kozak sequence (underlined). Use of this start site would generate a truncated CDH8 protein lacking the signal peptide, proprotein fragment, cadherin domain 1 and most of cadherin domain 2.
   Materials and Methods
   Lymphocyte Extraction and Metaphase Chromosome Preparation

Lymphocytes were extracted from 7 mls of patient blood (for storage and generation of EBV-transformed cell lines) using density gradient separation (Histopaque-1077, Sigma). In order to generate metaphase-arrested chromosomes for cytogenetic analysis 0.8 mls of patient blood were cultured for 71 hrs in medium containing phytohaemagglutinin (Peripheral Blood Medium, Sigma). The short-term cultures were treated with colcemid for one hour followed by a conventional fixing procedure. Fixed chromosomes were dropped onto microscope slides and stored for 1 week prior to use in FISH experiments.

Selection of YAC Clones for FISH Probe Synthesis

YAC clones were selected from the Whitehead/MIT map of the relevant chromosome in the cytogenetic intervals within which the breakpoints were adjudged to lie. YACs were obtained from the HGMP Resource Centre, Babraham Bioincubator, Babraham, Cambridge, UK (http://www.hgmp.mrc.ac.uk/). Clone DNA was prepared by standard methods and PCR amplified using primers designed against consensus sequence elements within the archetypal Alu repeat, Breen et al, 1992. This “Alu-PCR” gives a representative spread of non-repetitive sequence over the full length of the YAC and generates a better FISH probe than native YAC DNA. Alu-PCR was performed using the Expand Long Template PCR kit (Roche). Cycling conditions:94° C. 45 s, 55° C.-30 s, 68° C.-8 min: 35 cycles. 68° C.—10 min final extension.

Fluorescence in Situ Hybridisation (FISH) Protocol

Probe template DNA (pooled Alu-PCR products, BAC clone DNA, cosmid clone DNA or long-range PCR products) were labelled by nick translation and hybridised to patient metaphase spreads using standard FISH methods. Slides were counterstained with DAPI in Vectashield anti-fade solution (Vector laboratories). A Zeiss Axioskop fluorescence microscope with a chroma number 81000 or 830000 multi-spectral filter set was used to observe the chromosomal hybridisations. Images were captured using Vysis SmartCapture extension running within IP Lab spectrum or digital Scientific SmartCapture imaging software. FISH signals observed on derived chromosomes dictated the selection of further clones required to “walk” towards the breakpoint. Breakpoint-spanning FISH probes have signals on a normal chromosome and on both derived chromosomes.

Resolution of Breakpoint Position

BAC clones corresponding to positive YAC regions were arranged into contigs by consulting the Washington University FPC (http://www.genome.wustl.edu/gsc/human/Mapping/index.shtm 1), UCSC GoldenPath Draft Human Genome Browser (http://genome.ucsc.edu/goldenPath/hgTracks.html) and Ensembl (http://www.ensembl.org/) databases. BAC clones were supplied by BACPAC Resources, Oakland, Calif., USA (http://www.chori.org/bacpac/). Clone selection was biased to gene-containing BACs. Once a breakpoint-spanning BAC was identified, the position of the breakpoint in relation to candidate gene exons was determined by FISH probes generated from chromosome-specific library cosmids (HGMP Resource centre) or precisely positioned, repeat element-free long-range PCR products (Expand long range PCR kit, Roche; see below for primer sequences). Cycling conditions:94° C.-45 s, 52° C.-30 s, 68° C.-11 min: 35 cycles. 68° C.-15 min final extension. Cosmids were isolated by probing the appropriate chromosome-specific library filters (HGMP-RC) with isotopically labelled exon-specific PCR products.

### EXAMPLE 1

### Molecular Characterisation of Chromosomal Disruption and Identification of Disrupted Gene from Patient 1

FISH experiments on chromosome 3p13 had narrowed the location of the breakpoint to a region including the large gene SEMCAP3 (approximately 250 kb genomic extent). Two BAC clones were selected from the tiling diagram of BAC clones placed on the human genome map backbone (June 2002 release of the ‘BAC End Pairs’ track on the UCSC Genome Browser; http://qenome.cse.ucsc.edu/index.html?orq=Human). These were RPCI-11 606p16 and RPCI-11 94j25. By FISH, these BAC clones flanked the breakpoint (the former translocated to the derived chromosome 8 and the latter remained on the derived chromosome 3). The position of these two BAC clones indicated that the breakpoint lay within the large (200 kb) intron between exons 3 and 4 of the SEMCAP3 gene (see FIG. 2). Thus, the inventors inferred from these results that the SEMCAP3 gene was directly disrupted by the 3p13 translocation event and, as such, is a candidate gene for the psychiatric disorder exhibited by the patient.

Semcap3 (semaphorin cytoplasmic domain-associated protein) was originally identified in mouse as a gene encoding a protein that interacts with M-semF/Sema4c. Two forms, 3A and 3B, were submitted to the public nucleic acid sequence database (Wang & Strittmatter, 1999) but have yet to be published. It appears that 3b may be an artifactual sequence as it displays deletions in the sequence. Sema3a is identical in structure to the predicted human gene, KIAA1095 and the inventors refer to this sequence as human SEMCAP3. The yeast two-hybrid screen that isolated Sema3a/b also identified Sema1 and Sema2 as genes encoding proteins which interact with the cytoplasmic tail of the SEMA4C protein (Wang et al., 1999).

The purpose of these screening experiments was to elucidate cytoplasmic interactors with the transmembrane receptor, SEMA4C. This protein belongs to a large group of signalling proteins described as ‘semaphorins’. In the brain, these proteins are thought to play important roles in brain development through their action on axonal guidance and growth cone stability. Inagaki et al., (1995) showed that Sema4C is expressed in the developing mouse brain. One proposed explanation for the origin of psychiatric disorders (including the disorder exhibited by the patient described here) is the incorrect development of the brain, particularly the connections, projections and neural networks between brain subregions. With this in mind, semaphorins, and the proteins that interact with them (such as the SEMCAPS), become attractive candidate genes for the psychiatric disorders.

It is suspected that the PDZ domains (see FIG. 2) of the SEMCAP3 protein will be involved in protein-protein interactions (such as SEMA4C interaction) as they are in other proteins. The RING-finger domain of SEMCAP3 identifies it as belonging to a class of proteins known as ubiquitin ligases. Ubiquitin ligases specifically target proteins for ubiquitination and subsequent destruction in the proteasome pathway. Thus, SEMCAP3 may act to regulate the activity of other proteins (for instance, components of the semaphorin pathway) by targeting them for destruction. The ZF-TRAF/SINA domain is most likely an extension of the RING-finger domain.

FIG. 2 shows that the breakpoint would end SEMCAP3 transcription after the third exon on the derived chromosome 3 (there would still be one normal chromosome 3 and SEMCAP3 gene remaining in each nucleus). If transcription occurs on the derived chromosome 3 then the resulting translated protein product would be truncated; lacking part of the first PDZ domain and all subsequent amino acids in the C-terminal direction. It remains to be investigated if the psychiatric disorder in this patient results from N33 perturbation on one allele, the disruption of SEMCAP3 on one allele, the generation of an aberrantly functioning truncated SEMCAP3 from one allele or a combination of these.

Pulver et al. (1995) detailed schizophrenia linkage to chromosome 3p (albeit telomeric to SEMCAP3). However, two further studies have failed to replicate these findings in different populations (Maziade et al., 2001 & Hovatta et al., 1998).

### EXAMPLE 2

### Further Molecular Characterisation of Chromosomal Disruption and Identification of Disrupted Gene

In this case, primers corresponding to N33 3′UTR sequences and an STS, SHGC-12093 (Acc. No. G17275) were designed (see below for primer sequences). These PCR products were used to screen the chromosome 8 specific cosmid library (LA08). Among others, positive cosmids LA0854-H5 (3′ UTR) and LA08145-E3 (STS) were isolated and subsequently used in FISH experiments (see below for results).

3′UTR primers
Primer A: TGCCACGTGTTAGCAGAAAG
Primer B: TGCCTTTAACCAGATGAGGC
SHGC-12093 primers
Primer A: TCTTGTGGGTCACAATTAGGC
Primer B: TAAAAAGGTGCAGTTTCTTCAGC′.

The subject has schizoaffective disorder and a balanced reciprocal translocation between chromosomes 3 and 8. A 8p22 breakpoint-crossing YAC, 931_a_{—}1, was identified. This permitted a 8p22 breakpoint-crossing BAC RPCI-11 23j14 (acc. no. AC019292) to be found. This was shown to contain the 3′ end of the N33 gene (FIG. 6). Subsequently, FISH with cosmids LA0854-H5 and LA08145-E3 from the LANL chromosome 8 specific library (HGMP Resource Centre, Babraham, Cambridge, UK) flanked the breakpoint, placing it approximately 100 Kb from exon 11 of N33. N33 is related to a number of genes, human IAG2, Drosophila CG7830, C. elegans g304348 and two yeast proteins, OST3 and OST6 (see FIG. 8 for alignment of proteins). While the homologies between N33 and the yeast proteins are relatively weak, they share conserved cysteine residues and have the same locations for the four transmembrane domains as predicted by hydropathy plots. Ost3 and Ost6 are components of the oligosaccharyl transferase complex responsible for the addition of oligosaccharides to selected proteins. This has been backed up by protein structure prediction programs detailed in a recent report Fetrow et al, 2001.

The present inventors have identified an alternative start exon, herein identified as exon la (see FIGS. 5 & 6) to that in the public database, herein identified at exon 1b. Additionally they have identified a complex variation of splicing with the exons and proposed sequences of the transcripts, shown in FIGS. 5, 6 and 37 respectively. In view of the complex splice variations the C-terminal sequence of the various N3 splice forms is predicted to vary and this is shown in FIG. 9.

Because N33 lies within a linkage hotspot for schizophrenia (Gurling et al, 2001, Brzustowicz et al, 1999, Blouin et al, 1998, Kaufmann et al, 1998, Kendler et al, 1996, Pulver et al, 1995) the present inventors decided to carry out an association study on this gene. Three microsatellite markers (D8S549, N33 microsatellite and D8S1992

Microsatellites Used in Associated Study

D8S549

| | |
|---|---|
| Primer A: | AAATGAATCTCTGATTAGCCAAC |
| Primer B: | TGAGAGCCAACCTATTTCTACC |

N33 microsatellite

| | |
|---|---|
| Primer A: | AGGCTGAGTGCCAAAAAGTA |
| Primer B: | CTTTAAGCTTGCTATTTGAAGGC |

D8S1992

| | |
|---|---|
| Primer A: | TTCATCGTCTGAACCTGG |
| Primer B: | ACACATTTCCTCTATGTTGC) |

were chosen and used to type 25 mother-father-schizophrenic proband trios and 64 schizophrenic cases and G4 normal controls. The haplotypes derived from the trio study were examined for frequency bias in the case and control samples. Certain haplotypes are currently over-represented in the schizophrenic case genotypes compared to controls. Appropriate individuals with the haplotypes are currently being screened for mutations.

### EXAMPLE 3

### Molecular Characterisation of Chromosomal Disruption and Identification of Disrupted Gene from Patient 2

Psychiatric Evaluation

The subject (female) was approached and gave full, informed written consent for this study as one of a large cohort of people co-morbid for schizophrenia and mental retardation. Prior to investigation she was not known to have any abnormality of karyotype. She suffered from chronic schizophrenia and a mild degree of mental retardation (IQ between 65-70). The diagnosis of chronic schizophrenia was confirmed using SADS-L structured interview to generate DSM-IV and ICD-10 criteria, by a psychiatrist experienced in both general psychiatry and the psychiatry of mental retardation (WM). SADS can be reliably used in patients with mild mental retardation. Consensus diagnosis was reached on review by two psychiatrists (WM and DB). IQ scores were generated from WAIS-R and their stability shown by similar levels detected by psychological examination at different times throughout her life. There were no dysmorphic features in the subject. However the subject did suffer from bilateral deafness since childhood—a consequence of surgical operations on the mastoids. There was no family history of mental illness or mental retardation that could be ascertained. Other members of the family declined to participate in the study.

An initial G-banded karyotype of this patient indicated that the chromosome abnormality was complex (46, XX, ins (8;11) (q13;q23.3q24.2) inv (2) (p12q32.1) t (2;11) (q21.3;q24.2) der (2) (2qter→2q32.1::2p12→2q21.3::11a24.2→11qter) der(11) (11pter→11q23.3::2q21.3→2q32.1::2p12→2pter) der(8) (8pter→8q13::11q23.3→11q24.2::8q13→8qter)), involving a pericentric inversion of chromosome 2 coupled with rearrangements involving chromosomes 2, 8 and 11 (FIG. 13). FIG. 12 details the YAC and BAC FISH probes crossing or bracketing breakpoints on 2 and 11. Sequence in the locality of the breakpoints was assessed for gene content.

PCR Primers

Long-range PCR for FISH probe templates:

Int2-3 GRIK4a; CAGGAGGTCCTGTGAAGCTC,
Int2-3 GRIK4b; ACAGGGAAAGAAGCAAAGCA.

GRIK4 exon region-specific PCR: screening of chromosome 11 cosmid libraries:

| | |
|---|---|
| Ex1a/a′ | a; AAAGCTAAGCGCAGGTGTGT, |
| Ex1a/a′ | b; TTTCTGGGAGGCAACCATAG, |
| Ex1b | a; GCAGAGTTATGTCATGCCCA, |
| Ex1b | b; CCTGTGCAGCACTCTGATGT, |
| Ex2/3 | a; TTGAACCCAAGAGAACAGGG, |
| Ex2/3 | b; TCCCCTTCTCCTTCCAGTTT |

Cycling conditions:94° C.-2 min initial denaturation. 94° C.-1 min, 52° C.-1 min, 72° C.-75 s: 33 cycles. 72° C.-15 min final extension.

The 11q23.3 breakpoint is located at a locus containing a kainate-type ionotropic glutamate receptor (GRIK4, acc. S67803 & NM_{—}014619 (11), previous nomenclature KA1/EAA1). Cosmid FISH directed at the individual exons and an intron-specific long-range PCR product FISH (FIG. 15) positioned the breakpoint within the GRIK4 gene sequence; most likely immediately upstream of exon 2 (our nomenclature, FIG. 15). This was confirmed using a long-range PCR product FISH probe corresponding to the intron between exons 2 and 3 (FIG. 15). We also identified a GenBank EST (acc. BE388730, IMAGE clone ID:3613199) generating an alternative start-site resulting in an alternative cognate N-terminal peptide sequence (FIGS. 16 and 17). The position of a breakpoint anywhere between exons1a/a′/1b and exon 3 would truncate all putative transcript forms such that no receptor function could be encoded on the derived chromosome 11. Hence, the patient had only one intact GRIK4 allele.

Discussion

The present inventors identified a subject with comorbin schizophrenia with mild learning disability in whom chromosome translocation events have disrupted brain-expressed gene that are also functional disease candidates. Without wishing to be bound by theory it is hypothesised that the disruption of the GRIK4 gene by a chromosomal breakpoint (and the resulting reduced gene dosage) is the principal underlying cause of psychiatric disease in this patient.

The gene disrupted in this patient is both expressed in the brain and participates in key physiological processes in the CNS. Notably, the gene may be involved in the alteration of the strength of synaptic/neural transmission, a phenomenon known as long-term potentiation (LTP). LTP is postulated to underlie cognitive functions such as learning and memory. Moreover, cognitive testing has previously established that these functions are frequently affected in patients with schizophrenia.

GRIK4

Three classes of ionotropic glutamate receptors have been identified on the basis of their pharmacological profiles and sequence homologies; NMDA receptors, AMPA receptors and Kainate receptors. Functional Kainate receptors in vivo may be heteromeric, consisting of combinations of the low kainate agonist affinity (GLUR5, GLUR6 and GLUR7) and high-affinity subunits (GRIK4 and GRIK5) (Chittajallu et al, 1999; Lerma et al, 2001 and Werner et al, 1991). The subject with comorbid schizophrenia and mild learning disability possesses a complex chromosomal rearrangement. Of all the breakpoints studied in this patient only the GRIK4 gene is directly disrupted. This might be expected to modify kainate receptor channel properties by altering subunit stoichiometry.

The glutamate receptors are key initiators of synaptic LTP (Miller and Mayford, 1999). NMDA receptors are the principal mediators of LTP but recently presynaptic kainate receptor-dependent plasticity changes have been observed at mossy fibre synapses in the hippocampus (Contractor et al, 2001 and Lauri et al, 2001). Interestingly, an involvement of the glutamate neurotransmitter system in the pathophysiology of schizophrenia has been postulated. The “Glutamate Hypothesis” attempts to explain the psychotic symptoms that arise following administration of ionotropic glutamate receptor antagonists such as phencyclidine (PCP; “Angel Dust”) and ketamine (Goff and Nine, 1997). Several studies also point to changes, predominantly decreases, in glutamate receptor subunit expression (including kainate receptors) in the brains of schizophrenic patients (Ibrahim et al, and Meador-Woodruff, 2001). Similarly, Mohn et al, 1999 report that mutant mice with reduced NMDAR1 (another glutamate receptor) expression levels display schizophrenia-like behaviours.

As well as aberrant neurotransmission function in the adult, it has been suggested that neurodevelopmental deficits may contribute to schizophrenia. Neuroanatomical studies indicate statistically significant reduced volumes of brain regions, primarily the hippocampus, in schizophrenic and comorbid patients (Sanderson et al 1999 and Pearlson, 1999). GRIK4 is expressed in the amygdala, hippocampal formation (CA3 pyramidal and dentate granule cells) and entorhinal cortex. Glutamate receptors might mediate brain development through the activity-dependent refinement of neuronal connections.

The present subject was clinically diagnosed as having schizophrenia coupled with mild learning disability. It may be the case that causative gene mutations in comorbid patients lead to a severe phenotype or have more profound downstream effects than gene mutations in patients with schizophrenia alone (i.e. the comorbid state represents the severest form of schizophrenia (Doody et al, 1998)). A second possibility is that the gene mutation gives rise to the learning disability component of the illness through an independent effect on brain development. The manner in which the mutated genotype gives rise to the observed phenotype (via functional or developmental mechanisms) is a key issue in molecular neurobiology, particularly in the characterisation of mouse “knockout” mutants (Mayford et al, 1995).

A large number of publications detail family and population-based linkage studies carried out to identify psychiatric illness susceptibility loci. The results have not been conclusive perhaps indicating the presence of confounding factors such as population stratification, incomplete penetrance, genetic heterogeneity and uncertain mode of inheritance. Nevertheless, GRIK4 lies at the edge of a schizophrenia linkage region described in a recent publication (Gurling et al, 2001). The most centromeric marker exhibiting linkage to schizophrenia in this paper, D11S925, is located within an intron at the 3′ end of GRIK4.

### EXAMPLE 4

### Molecular Characterisation of Chromosomal Disruption and Identification of Disrupted Gene from Patient 3

Fine FISH Mapping of the Breakpoint with Cosmid Clones

PCR products corresponding to regions in or near hNPAS3 exons 4, 5 and 6 were obtained using the following primers under standard PCR conditions (Exon 4-i ACAACCATTCTGGGAACAGC, Exon 4-ii GTGTAGGGAAAGCCATCCAA, Exon 5-i TCTTTTTCCTGCAGTCCCTG, Exon 5-ii CTCCAAATGACTCCTGCCAT, Exon 6-i GCCTCTGCCATAGATTTTGC, Exon 6-ii TTCCTTCCCACCCTTTCTCT). Probes were created by random-primed labelling of PCR products with radioactive dCTP; these were used to screen a LANL chromosome 14-specific cosmid library (LA14NC01 obtained from the UK HGMP Resource Centre, Hinxton, Cambridge) using hybriding conditions set out in Church and Gilbert (1986). Positive clones (exon; LA1431-G5, exon 5: LA14123-C4 and exon 6; LA1487-D9) were prepared by a standard alkaline lysis protocol and taken through FISH analysis as above.

Results

Metaphase spreads from EBV-transformed cell lines were analysed by Fluorescence in situ Hybridisation (FISH) using successively smaller DNA probes. A breakpoint spanning BAC clone was obtained by FISH screening (RPCI-11 BAC 1078i14, acc. no. AL161851). EST sequences were examined in the genomic DNA flanking the breakpoint in order to identify potential transcripts in the locality. A number of ESTs were identified which had been annotated as containing homologous sequence to the conserved “PAS” domain present in a large number of genes (Gu et al, 2000). A search of such genes revealed that the most closely related gene encoded a mouse brain-expressed transcript, neuronal pas domain protein 3 (NPAS3 (MOPG), acc. no. AF137871; hereafter referred to as mNPAS3). Nucleotide homology to the mNPAS3 cDNA within human genomic DNA BAC clone sequences at 14q13 using the BLAST algorithm identified 12 exons corresponding to the human orthologue of mNPAS3 (hNPAS3) distributed over a genomic region of approximately 800-900 Kb making it among the largest gene loci in the human genome (FIG. 23). Subsequently, full length hNPAS3 cDNA sequences have been submitted by two other groups to GenBank/EMBL with the accession numbers, AB054575 and AF164438, although these have differences to the mouse splice-form in the 5′ exons. This is due to the presence of two alternative transcription start sites employed in both human and mouse genes. This was confirmed by analysis of published cDNA and EST sequences coupled with further sequencing of corresponding IMAGE clones. These splice variants are highlighted in FIGS. 18, 30 and 23.

The ratio of fluorescent signals on the derived chromosomes 9 and 14 from the breakpoint-spanning BAC probe, 1078i14, indicated that the breakpoint was located at the centromeric end of the BAC. This is the location of exon 5 of the gene. Exon 4-, 5- and 6-containing cosmids were isolated and used as FISH probes to provide definitive proof of the location of the breakpoint and confirmation that a full-length transcript (and hence protein) cannot be synthesized on the derived chromosome 14. An exon 5-containing cosmid (see FIG. 23) spanned the breakpoint. Subsequently a long-range PCR product-derived FISH probe corresponding to exon 5 indicated that the breakpoint lay upstream of exon 5.

Long-range PCR primers-NPAS3 exon 5
a) ccagcttgtatgtggtgtgg
b) ttactcccagtgcccattgt.

Discussion

A FISH-based approach has shown that the gene, NPAS3, is disrupted by a chromosomal rearrangement present in a mother and daughter who suffer from comorbid schizophrenia and learning disability respectively. NPAS3 is a brain expressed transcription factor of the basic helix-loop-helix PAS domain class which includes members such as AHR and ARNT.

Neuronal pas3 (NPAS3) was originally cloned in the mouse (Brunskill et al, 1999) on the basis of its sequence homology with other PAS domain proteins. Its expression has been characterised in the developing mouse embryo where high levels are seen in the neural tube, neuroepithelium and, later, the neopallial layer of the cortex. Non-neural expression was also observed in the heart, limb and kidney. In the mouse, NPAS1 (human chromosomal location, 19q13) is expressed in deep pyramidal cortex cells, hippocampus and amygdala (Zhou et al., 1997). NPAS2 (human chromosomal location, 2q13) is expressed in the cortex, hippocampus and thalamus. Lower levels were also seen in spinal cord, intestines and uterus. NPAS2 was also recently deleted in mice by homologous recombination (Garcia et al., 2000) leading to deficits in cued and contextual memory. In addition NPAS2 appears to have a role in cellular energy state monitoring and the circadian rhythm pathway (Reick et al, 2001 and Rutter et al, 2001). The translocation event described herein disrupts the gene between exons 4 and 5. If transcription occurred at this disrupted locus, a truncated protein would result containing only the bHLH domain. It is conceivable that this protein would have a dominant negative effect on wild-type NPAS3 protein (or any other heterodimeric protein partner) through the creation of non-functional dimers (see FIG. 24 for explanatory diagram). This would result in a potentially more severe or penetrant phenotype than a conventional point mutation. Two examples where bHLH-PAS proteins have been altered through loss of the C-terminal PAS domain (one experimentally, the other in a patient with a chromosome translocation) have resulted in probable dominant negative action (Maemura et al, 1999, Holder jr. et al, 2000).

Mutations in this gene in karyotypically normal individuals would not be expected to have as severe or penetrant effects as those observed in the two t(9;14) patients.

Sequence comparison between hNPAS3 and other members of the NPAS sub-family show that homologies are largely restricted to the N-terminal end of the protein; the location of a basic helix-loop-helix and PAS domains. The greatest homology is with NPAS1, then NPAS2 and other PAS domain-containing proteins (data not shown). An alignment of the cognate human (conceptually translated from the splice-form containing exons 1-12) and mouse NPAS3 proteins reveals near identity over the N-terminal half of the protein but increased divergence at the C-terminal end. This is particularly the case for two stretches where 5 and 7 amino acids, respectively, have been gained in the human orthologue (Fig.21). These correspond to two poly-glycine tracts present within exon 12 (of 11 and 10 residues respectively). Such tracts can be indicative of slipped strand mispairing whereby trinucleotide repeats are aberrantly expanded or deleted. Where they occur in coding sequence, increases in the number of trinucleotide repeats can have a pathological effect on protein function (e.g. Huntington disease and Spino-cerebellar ataxia 1). Another feature of such repeats is their unstable nature between generations: a lowering of the age of onset of a disease from generation to generation (anticipation) can often be directly linked to an increase in the number of repeat units.

Exon 12 (coding for the C-terminus of the protein) is also noteworthy because of the extremely high density of CpG dinucleotides (in humans and mouse); a feature that abruptly ends at the junctions with flanking intronic/3′ sequences. This “CpG island” is unusual because it is both transcribed and also located at the 3′ rather than 5′ end of the gene. The significance of this in terms of potential transcriptional control by methylation or susceptibility to mutation is as yet unknown. However, the high level of G and C bases creates a bias in amino acid composition such that alanine, glycine, histidine and proline are over-represented. This may explain the presence and expansion of the poly-glycine tracts in Npas3.

14q13 is also the site of linkage to Fahr's syndrome (idiopathic basal ganglia calcification; IBGC) as determined from analysis of families (Geschwind et al, 1999). Fahr's syndrome symptoms are often accompanied by psychoses such as schizophrenia. Thus, it may be the case that NPAS3 is also the gene responsible for Fahr's syndrome.

### EXAMPLE 5

### Molecular Characterisation of Chromosomal Disruption and Identification of Disrupted Gene from Patient 4

Psychiatric Evaluation

The subject (male) is the proband in a family segregating a t(1;16) balanced reciprocal translocation. He gave full informed consent to the study. His diagnosis of chronic schizophrenia was confirmed by SADS-L structured interview and a consensus reached by two psychiatrists (WM and DB). He does not have mental retardation. Other members of his near family also gave consent to participate in this study, none of whom had current mental illness (several are below the age of risk for psychiatric illness). There was also a history of mental illness (major depressive disorder) in members of the extended family who were known to be translocation carriers, but they could not be approached for confirmation at the time of the current study. An unrelated individual (now deceased) with DSM-IV chronic schizophrenia without learning disability also had a t(1;16) balanced translocation with the same breakpoints (at the resolution of G-banding).

PCR Primers

Long-range PCR for FISH probe templates: PDE4B3a; GTCAGACAAATCCAAATGGAGAG, PDE4B3b; CTTTCTCCTGTCACTTTCCTTCA. Cycling conditions:94° C.-2 min initial denaturation. 94° C.-1 min, 52° C.-1 min, 72° C.-75 s: 33 cycles. 72° C.-15 min final extension.

The balanced translocation, t(1;16) (p31.2;q21) in this family results in two breakpoints (FIG. 33). Genomic sequence at 16q21 is not complete. The only known gene in the vicinity of the breakpoint region is Cadherin 8 (CDH8, acc. AB035305).

In contrast, on chromosome 1p31.2 FISH identified two non-overlapping BAC clones (RP11-433N2, acc. AL513493 and RP11-442I1, acc. AL391359) which reside on either side of the breakpoint in this patient. The breakpoint-containing genomic region between these two BAC clones has yet to be sequenced (see FIG. 32). Database annotation of the two BAC clones together with BLAST mapping of exons onto genomic sequence indicated that this locus contains a cAMP phosphodiesterase gene, PDE4B. Two cDNAs corresponding to longer transcript forms of this gene (denoted PDE4B1, acc. L20966 and PDE4B3, acc. U85048, respectively) have been previously characterised (Bolger et al, 1994; Huston et al, 1997). Long-range PCR product FISH (FIG. 32) confirmed that the PDE4B1 transcript is directly disrupted by the breakpoint (although additional position-effect perturbation of PDE4B3 expression cannot be ruled out). Huston et al. (1997) have previously shown that the PDE4B1 transcript encodes an alternative N-terminal peptide sequence. In addition, they demonstrated that only this form is expressed in the brain. It is therefore predicted that this patient will have a reduction in the levels of functional PDE4B in the brain.

Discussion

The present inventors have identified a subject with DSMIV chronic schizophrenia in whom chromosome translocation events have disrupted brain-expressed genes that are also functional disease candidates. Without wishing to be bound by theory it is hypothesised that the disruption of the PDE4B gene by a chromosomal breakpoint (and the resulting reduced gene dosage) is the principal underlying cause of psychiatric disease in this patient.

The gene disrupted in this patient is both expressed in the brain and participates in key physiological processes in the CNS. Notably, the gene may be involved in the alteration of the strength of synaptic/neural transmission, a phenomenon known as long-term potentiation (LTP). LTP is postulated to underlie cognitive functions such as learning and memory. Moreover, cognitive testing has previously established that these functions are frequently affected in patients with schizophrenia.

PDE4B

Stimulation of the G protein coupled receptor/heterotrimeric G protein pathway results in the synthesis of the secondary messenger, cAMP, by members of the adenylyl cyclase family of enzymes. This secondary messenger triggers a well-characterised signalling cascade that is principally mediated by cAMP-dependent protein kinase A (PKA) and cAMP-resposive transcription factor, CREB, both of which have been implicated in the molecular pathways of LTP (Abel & Latal, 2001). cAMP signalling is attenuated by its breakdown by members of the phosphodiesterase enzyme family. Four members of the PDE4 sub-family of cAMP phosphodiesterases have been identified to date (PDE4A-PDE4D). These four genes are the human homologues of the Drosophila learning and memory mutant gene, Dunce. The long form of the PDE4B protein, PDE4B1, is the only splice form with brain expression and the present inventors have shown that it is disrupted in the subject. Anti-PDE4B antibodies revealed expression within the inferior olive, the hypothalamus, the ventral striatum, the cerebellar molecular layer, globus pallidus, nucleus accumbens and substantia nigra (Cherry & Davis, 1999). The authors of this expression study suggested that PDE4B expression strongly correlates with brain areas underlying reward and affect in mammals. In addition, PDE4 proteins are recognised as the molecular targets for Rolipram, a drug with anti-depressant effects. Rolipram inhibition of PDE4 activity has been shown to improve long-term hippocampal LTP and spatial memory in mice (Barad et al, 1998 and Bach et al, 1991). The (heterozygous) disruption to PDE4B1 described here may be equivalent to 50% reduction of protein product in the brain. This could result in a greater cAMP half-life and a concomitant increase in the activation of downstream cAMP targets.

In addition, the disruption to PDE4B shows reduced penetrance as not all translocation carriers present with psychiatric illness (although all members of the extended family with psychiatric illness possess the translocation karyotype; data not shown).

### EXAMPLE 6

### Molecular Characterisation of Chromosomal Disruption and Identification of Disrupted Gene from Patient 4

FISH experiments on chromosome 16q21 had narrowed the location of the breakpoint to a region including the large gene CDH8 (approximately 400 kb genomic extent). Three BAC clones were selected from the tiling diagram of BAC clones placed on the human genome map backbone (June 2002 release of the ‘BAC End Pairs’ track on the UCSC Genome Browser; http://qenome.cse.ucsc.edu/index.html?orq=Human). These were RPCI-11 599c11, RPCI-11 875e12 and RPCI-11 685m21. By FISH, these BAC clones flanked the breakpoint (the first two translocated the derived chromosome 1 whereas the third remained on the derived chromosome 16). The position of these three BAC clones indicated that the breakpoint lay within the large (100 kb) intron between exons 1 and 2 of the CDH8 gene (see FIG. 2). Thus, the inventors inferred from these results that the CDH8 gene was directly disrupted by the 16q21 translocation event and, as such, is a candidate gene for the psychiatric disorder exhibited by the patient. The similar disruption of the PDE4B gene on chromosome 1 and their relative orientations on the two chromosomes raised the possibility that the derived chromosomes (the two chromosomes resulting from the translocation: der(1) and der(16)) could transcribe fusion/hybrid genes. This has been frequently seen in cases where a translocation gives rise to susceptibilty to cancers. In essence, the translocation in the proband resulted in an exchange of the two genes' promoter and first exon sequences. On the der(1) the promoter and first exon of the CDH8 gene are juxtaposed to exon 2 and downstream of the PDE4B gene (see FIG. 33). However, the reading frames of these two gene segments are not the same, resulting in a prematurely truncated peptide with only the signal peptide, proprotein fragment and a small portion of the cadherin domain contained within (see FIG. 37a). This would be expressed in the same cell types/tissues as the normal CDHB gene but the functional/pathological significance of this small peptide is not clear at the current time. On the der(16) the PDE4B promoter and exon la are juxtaposed to exon 2 and downstream of the CDH8 gene (see FIG. 33). Exon 1a of PDE4B does not contain a translation start-site so the reading frame compatabilities of the putative fused transcript are not an issue. However, exon 2 and downstream of the CDH8 gene contain several ATG start-sites which could be employed by translational machinery to generate peptide sequences. In two of the reading frames, any generated peptides would be small and probably of no consequence. The third reading frame (the normal CDH8 reading frame, see FIG. 5b) contains three ATG start-sites early on, with the second of these forming a very good match to the canonical Kozak sequence found at most translation start-sites (CCAxxATGG). If this one is used then the resulting peptide will be identical to normal CDH8 protein but lacking the N-terminal portion encoding the signal peptide, proprotein fragment, the first cadherin domain and most of the second cadherin domain. Although the bulk of the peptide sequence is as the normal CDH8 protein, the lack of the N-terminal sequences may prevent the protein from entering the Golgi/ER subcellular compartments—a process that is required for the correct insertion in/trafficking to the cell membrane. The functional/pathological consequence of the presence of this truncated form of the CDH8 protein in the cytoplasm of tissues where the long form of the PDE4B gene is expressed is uncertain at this point.

In summary, the psychiatric illness seen in the proband, and other members of the family, may be the result of one (or a combination) of the following circumstances: the loss (through disruption) of one allele of PDE4B, the loss (through disruption) of one allele of CDHB or the generation of potentially pathological fusion polypeptides.

Cadherin-8 was first cloned in humans (Tanihara et al., 1994) and later in mouse (Munro et a., 1996) and rat (Kido et al., 1998). Sequence analysis immediately placed the gene product within the large family of membrane-spanning proteins with extracellular cadherin domains thought to mediate calcium-dependent homophilic interactions between adjacent cells. As such, the cadherins are members of the functionally defined group of cell adhesion proteins.

CDH8 is a member of the Type II, or atypical, cadherins which are defined by the lack of an extracellular tripeptide motif, HAV, possibly involved in the binding specificity of Type I cadherins. FIG. 2 illustrates the structure of CDH8 protein which includes an extracellular domain containing 5 copies of the cadherin domain, a membrane spanning domain and a C-terminal cytoplasmic tail. The cytoplasmic tail is thought to signal the presence of interactions to the intracellular compartment by mediating receptor clustering through interaction with the proteins such as β-catenin, α-catenin and, eventually, the cytoskeletal proteins, actin and α-actinin. In this way, adhesion to adjacent cells can affect the cytoarchitecture of the cell and may even play a role in cell motility.

The two principal roles of neuronal cadherirns are thought to be in the mediation of certain developmental pathways in the brain and the regulation of synaptic function. The homophilic nature of cadherin interaction (i.e. CDH8 proteins preferentially bind to other CDH8 proteins) has prompted the hypothesis that cadherins are responsible for the aggregation or interconnection of similar cells within an organ. This has been shown to be the case in the brain where CDHB expression has been shown to be restricted to particular subregions and even neuronal patches (Redies, Bishop, Rubenstein, Korematsu X 2).

The major cadherin in the brain, N-cadherin (encoded by CDH2), has been implicated in synaptic long-term potentiation (LTP): the mechanism thought to underlie learning and memory on the brain (e.g. Huntley et al., 2002 & Bozdagi et al., 2000,). Other cadherins may also play a part in this process (Uemura, 1998 & Tang et al., 1998). In essence, cadherins seem to form physical bridges across the synaptic cleft which may modify synaptic efficacy and/or spine morphology (two features of neurons demonstrated to change after the induction of LTP).

Interestingly, two of the hypotheses used to explain the origins of psychiatric illness are, firstly, the occurrence of abnormal brain development and, secondly, the existence of deficits in cellular pathways manifested as poor performance in certain cognitive/memory tasks. The two roles of neuronal cadherins seem to closely mirror these two hypotheses suggesting that CDH8 is a good functional candidate for psychiatric illness.

### REFERENCES

1. Berrettini W. H. (2000) Genetics of psychiatric disease. Annu. Rev. Med. 51:465-79.
2. Lai C. S, Fisher S. E, Hurst J. A, Vargha-Khadem F, Monaco A. P. (2001) A forkhead-domain gene is mutated in a severe speech and Language disorder. Nature. 413(6855):519-23.
3. Kleinjan D. J, van Heyningen V. (1998) Position effect in human genetic disease. Hum Mol Genet. 7(10):1611-8.
4. Brennan J, Skarnes W. C. (1999) Gene trapping in mouse embryonic stem cells. Methods Mol. Biol. 97:123-38.
5. Breen M, Arveiler B, Murray I, Gosden J. R, Porteous D. J. (1992) YAC mapping by FISH using Alu-PCR-generated probes. Genomics. 13(3):726-30.
6. Fetrow J. S, Siew N, Di Gennaro J. A, Martinez-Yamout M, Dyson H. J, Skolnick J. (2001) Genomic-scale comparison of sequence- and structure-based methods of function prediction: does structure provide additional insight? Protein Sci. 2001 May;10(5):1005-14.
7. Gurling H. M, Kalsi G, Brynjolfson J, Sigmundsson T, Sherrington R, Mankoo B. S, Read T, Murphy P, Blaveri E, McQuillin A, Petursson H, Curtis D. (2001) Genomewide genetic linkage analysis confirms the presence of susceptibility loci for schizophrenia, on chromosomes 1q32.2, 5q33.2, and 8p21-22 and provides support for linkage to schizophrenia, on chromosomes 11q23.3-24 and 20q12.1-11.23. Am J Hum Genet. Mar;68(3):661-73.
8. Brzustowicz L. M, Honer W. G, Chow E. W, Little D, Hogan J. Hodgkinson K, Bassett A S. (1999) Linkage of familial schizophrenia to chromosome 13q32. Am J Hum Genet. October;65(4):1096-103.
9. Blouin J. L, Dombroski B. A, Nath S. K, Lasseter V. K, Wolyniec P. S, Nestadt G, Thornquist M, Ullrich G, McGrath J, Kasch L, Lamacz M, Thomas M. G, Gehrig C, Radhakrishna U, Snyder S. E, Balk K. G, Neufeld K, Swartz K. L, DeMarchi N, Papadimitriou G. N, Dikeos D. G, Stefanis C. N, Chakravarti A, Childs B, Pulver A. E, et al. (1998) Schizophrenia susceptibility loci on chromosomes 13q32 and 8p21. Nat Genet. September;20(1):70-3.
10. Kaufmann C. A, Suarez B, Malaspina D, Pepple J, Svrakic D, Markel P. D, Meyer J, Zambuto C. T, Schmitt K, Matise T. C, Harkavy Friedman J. M, Hampe C, Lee H, Shore D, Wynne D, Faraone S. V, Tsuang M. T, Cloninger C. R. (1998) NIMH Genetics Initiative Millenium Schizophrenia Consortium: linkage analysis of African-American pedigrees. Am J Med Genet. July 10;81(4):282-9.
11. Kendler K. S, MacLean C. J, O'Neill F. A, Burke J, Murphy B, Duke F, Shinkwin R, Easter S. M, Webb B. T, Zhang J, Walsh D, Straub (1996) Evidence for a schizophrenia vulnerability locus on chromosome 8p in the Irish Study of High-Density Schizophrenia Families. Am J Psychiatry. December;153(12):1534-40.
12. Pulver A. E, Lasseter V. K, Kasch L, Wolyniec P, Nestadt G, Blouin J. L, Kimberland M, Babb R, Vourlis S, Chen H, et al. (1995) Schizophrenia: a genome scan targets chromosomes 3p and 8p as potential sites of susceptibility genes. Am J Med Genet. June 19;60(3):252-60.
13. MacGrogan D, Levy A, Bova G. S, Isaacs W. B and Bookstein R. Structure and methylation-associated silencing of a gene with a homozygously deleted region of himan chromosome band 8p22. Genomics 35 (1), 55-65 (1996).
14. Brennan J, Skarnes W. C. (1999) Gene trapping in mouse embryonic stem cells. Methods Mol. Biol. 97:123-38.
15. Doody G. A, Johnstone E. C, Sanderson T. L, Owens D. G, Muir W. J. (1998) “Pfropfschizophrenie” revisited. Schizophrenia in people with mild learning disability. Br J Psychiatry. 173:145-53.
16. Sanderson T. L, Doody G. A, Best J, Owens D. G, Johnstone E. C. (2001) Correlations between clinical and historical variables, and cerebral structural variables in people with mild intellectual disability and schizophrenia. J Intellect Disabil Res. 45(Pt 2):89-98.
17. Kamboj R. K, Schoepp D. D, Nutt S, Shekter L, Korczak B, True R. A, Rampersad V, Zimmerman D. M, Wosnick M. A. (1994) Molecular cloning, expression, and pharmacological characterization of humEAA1, a human kainate receptor subunit. J Neurochem. 62(1):1-9.
18. Martin S. J, Grimwood P. D, Morris R. G. (2000) Synaptic plasticity and memory: an evaluation of the hypothesis. Annu Rev Neurosci. 23:649-711.
19. Kuperberg G, Heckers S. (2000) Schizophrenia and cognitive function. Curr Opin Neurobiol. 10(2):205-10.
20. Elvevag B, Goldberg T. E. (2000) Cognitive impairment in schizophrenia is the core of the disorder. Crit Rev Neurobiol. 14(1):1-21.
21. Blackwood D. H, Glabus M. F, Dunan J, O'Carroll R. E, Muir W. J, Ebmeier K. P. (1999) Altered cerebral perfusion measured by SPECT in relatives of patients with schizophrenia. Correlations with memory and P300.Br J Psychiatry. 175:357-366.
22. Chittajallu R, Braithwaite S. P, Clarke V. R, Henley J. M (1999) Kainate receptors: subunits, synaptic localization and function. Trends Pharmacol Sci. 20(1):26-35.
23. Lerma J, Paternain A. V, Rodriguez-Moreno A, Lopez-Garcia J. C. (2001) Molecular physiology of kainate receptors. Physiol Rev. 81(3):971-98.
24. Werner P, Voigt M, Keinanen K, Wisden W, Seeburg P. H (1991) Cloning of a putative high-affinity kainate receptor expressed predominantly in hippocampal CA3 cells. Nature. 351:742-4.
25. Miller S, Mayford M. (1999) Cellular and molecular mechanisms of memory: the LTP connection. Curr Opin Genet Dev. 9(3):333-7.
26. Contractor A, Swanson G, Heinemann S. F. (2001) Kainate receptors are involved in short- and long-term plasticity at mossy fiber synapses in the hippocampus. Neuron. 29(1):209-16.
27. Lauri S. E, Bortolotto Z. A, Bleakman D, Ornstein P. L, Lodge D, Isaac J. T, Collingridge G. L. (2001) A Critical Role of a Facilitatory Presynaptic Kainate Receptor in Mossy Fiber LTP. Neuron. November 20;32(4):697-709.
28. Goff D. C, Wine L. (1997) Glutamate in schizophrenia: clinical and research implications. Schizophr Res. 27(2-3):157-68.
29. Ibrahim H. M, Hogg A. J Jr, Healy D. J, Haroutunian V, Davis K. L, Meador-Woodruff J. H. (2000) Ionotropic glutamate receptor binding and subunit mRNA expression in thalamic nuclei in schizophrenia. Am J Psychiatry. 157(11):1811-23.
30. Meador-Woodruff J. H, Davis K. L, Haroutunian V. (2001) Abnormal kainate receptor expression in prefrontal cortex in schizophrenia. Neuropsychopharmacology. 24(5):545-52.
31. Mohn A. R, Gainetdinov R. R, Caron M. G, Koller B. H. (1999) Mice with reduced NMDA receptor expression display behaviors related to schizophrenia. Cell. 98(4):427-36.
32. Sanderson T. L, Best J. J, Doody G. A, Owens D. G, Johnstone E. C (1999) Neuroanatomy of comorbid schizophrenia and learning disability: a controlled study. Lancet. 354(9193):1867-71.
33. Pearlson G. D. (1999) New insights on the neuroanatomy of schizophrenia. Curr Psychiatry Rep. 1(1):41-5.
34. Li Y, Erzurumlu R. S, Chen C, Jhaveri S, Tonegawa S (1994) Whisker-related neuronal patterns fail to develop in the trigeminal brainstem nuclei of NMDAR1 knockout mice. Cell. 76(3):427-37.
35. Iwasato T, Datwani A, Wolf A. M, Nishiyama H, Taguchi Y, Tonegawa S, Knopfel T, Erzurumlu R. S, Itohara S (2000) Cortex-restricted disruption of NMDAR1 impairs neuronal patterns in the barrel cortex. Nature. 406(6797):726-31.
36. Abel T, Lattal K. M. (2001) Molecular mechanisms of memory acquisition, consolidation and retrieval. Curr Opin Neurobiol. 11(2):180-7.
37. Mayford M, Abel T, Kandel E. R. (1995) Transgenic approaches to cognition. Curr Opin Neurobiol. 5(2):141-8.
38. Chumakov et al. (1992). Continuum of overlapping clones spanning the entire human chromosome 21q. Nature. 359, 380-386.
39. Osoegawa K, Mammoser A. G, Wu C, Frengen E, Zeng C, Catanese J. J, de Jong P. J. (2001) A bacterial artificial chromosome library for sequencing the complete human genome. Genome Res. 11(3):483-96.
40. Kozak M. (1986) Point mutations define a sequence flanking the AUG initiator codon that modulates translation by eukaryotic ribosomes. Cell. 44(2):283-92.
41. Bolger G. B, Rodgers L, Riggs M. (1994) Differential CNS expression of alternative mRNA isoforms of the mammalian genes encoding cAMP-specific phosphodiesterases. Gene. 149(2):237-44.
42. Huston E, Lumb S, Russell A, Catterall C, Ross A. H, Steele M. R, Bolger G. B, Perry M. J, Owens R. J, Houslay M. D. (1997) Molecular cloning and transient expression in COS7 cells of a novel human PDE4B cAMP-specific phosphodiesterase, HSPDE4B3. Biochem J. 328 ( Pt 2):549-58.
43. Cherry J. A, Davis R. L. (1999) Cyclic AMP phosphodiesterases are localized in regions of the mouse brain associated with reinforcement, movement, and affect. J Comp Neurol. 407(2):287-301.
44. Barad M, Bourtchouladze R, Winder D. G, Golan H, Kandel E. (1998) Rolipram, a type IV-specific phosphodiesterase inhibitor, facilitates the establishment of long-lasting long-term potentiation and improves memory. Proc Natl Acad Sci USA. 95(25):15020-5.
45. Bach M. E, Barad M, Son H, Zhuo M, Lu Y. F, Shih R, Mansuy I, Hawkins R. D, Kandel E. R. (1999) Age-related defects in spatial memory are correlated with defects in the late phase of hippocampal long-term potentiation in vitro and are attenuated by drugs that enhance the cAMP signaling pathway. Proc Natl Acad Sci USA. 96(9):5280-5.
46. Gu Y. Z, Hogenesch J. B, Bradfield C. A. The PAS superfamily: sensors of environmental and developmental signals. (2000) Annu Rev Pharmacol Toxicol.;40:519-61.
47. Brunskill E. W, Witte D. P, Shreiner A. B, Potter S. S. (1999) Characterization of npas3, a novel basic helix-loop-helix PAS gene expressed in the developing mouse nervous system. Mech Dev. November;88(2):237-41.
48. Zhou Y. D, Barnard M, Tian H, Li X, Ring H. Z, Francke U, Shelton J, Richardson J, Russell D. W, McKnight S. L. (1997) Molecular characterization of two mammalian bHLH-PAS domain proteins selectively expressed in the central nervous system. Proc Natl Acad Sci USA. January 21;94(2):713-8.
49. Garcia J. A, Zhang D, Estill S. J, Michnoff C, Rutter J, Reick M, Scott K, Diaz-Arrastia R, McKnight S. L. (2000) Impaired cued and contextual memory in NPAS2-deficient mice. Science. June 23;288(5474):2226-30.
50. Reick M, Garcia J. A, Dudley C, McKnight S. L. (2001) NPAS2: an analog of clock operative in the mammalian forebrain. Science. July 20;293(5529):506-9.
51. Rutter J, Reick M, Wu L. C, McKnight S. L. (2001) Regulation of clock and NPAS2 DNA binding by the redox state of NAD cofactors. Science. July 20;293(5529):510-4.
52. Maemura K, Hsieh C. M, Jain M. K, Fukumoto S. Layne M. D, Liu Y, Kourembanas S, Yet S. F, Perrella M. A, Lee M. E. (1999) Generation of a dominant-negative mutant of endothelial PAS domain protein 1 by deletion of a potent C-terminal transactivation domain. J Biol Chem. October 29;274(44):31565-70.
53. Holder J. L Jr, Butte N. F, Zinn A. R. (2000) Profound obesity associated with a balanced translocation that disrupts the SIM1 gene. Hum Mol Genet. January 1;9(1)101-8.
54. Geschwind D. H, Loginov M, Stern J. M. (1999) Identification of a locus on chromosome 14q for idiopathic basal ganglia calcification (Fahr disease). Am J Hum Genet. September 1999;65(3):764-72.
55. Wang L H, Kalb R G, Strittmatter S M. (1999) A PDZ protein regulates the distribution of the transmembrane semaphorin, M-SemF. J Biol Chem. 1999 274(20):14137-46.
56. Inagaki S, Furuyama T, Iwahashi Y. (1995) Identification of a member of mouse semaphorin family. FEBS Lett. 370(3):269-72.
57. Mazlade M, Rol M A, Rouillard E, Bissonnette L, Fournier J P, Roy A, Garneau Y, Montgrain N, Potvin A, Cliche D, Dion C, Wallot H, Fournier A, Nicole L, Lavallee J C, Merette C. (2001) A search for specific and common susceptibility loci for schizophrenia and bipolar disorder: a linkage study in 13 target chromosomes. Mol Psychiatry. 6 (6) :684 -93.
58. Hovatta I, Lichtermann D, Juvonen H, Suvisaari J. Terwilliger J D, Arajarvi R, Kokko-Sahin M L, Ekelund J, Lonnqvist J, Peltonen L. (1998) Linkage analysis of putative schizophrenia gene candidate regions on chromosomes 3p, 5q, 6p, 8p, 20p and 22q in a population-based sampled Finnish family set. Mol Psychiatry. 3(5):452-7.
59. Shimoyama Y, Tsujimoto G, Kitajima M, Natori M. (2000) Identification of three human type-II classic cadherins and frequent heterophilic interactions between different subclasses of type-II classic cadherins. Biochem J. 349(Pt 1):159-67.
60. Korematsu K, Goto S, Okamura A, Ushio Y. (1998) Heterogeneity of cadherin-8.expression in the neonatal rat-striatum: comparison with striatal compartments. Exp Neurol-154(2):531-6.
61. Korematsu K, Nishi T, Okamura A, Goto S, Morioka M, Hamada J, Ushio Y. (1998) Cadherin-8 protein expression in gray matter structures and nerve fibers of the neonatal and adult mouse brain. Neuroscience. 87(1):303-15.
62. Kido M, Obata S, Tanihara H, Rochelle J M, Seldin M F, Taketani S, Suzuki S T. (1998) Molecular properties and chromosomal location of cadherin-8. Genomics. 1998 48(2):186-94.
63. Korematsu K, Redies C. (1997) Expression of cadherin-8 mRNA in the developing mouse central nervous system. J Comp Neurol.387(2):291-306.
64. Korematsu K, Redies C. (1997) Restricted expression of cadherin-8 in segmental and functional subdivisions of the embryonic mouse brain. Dev Dyn. 208(2):178-89.
65. Tanihara H, Sano K, Heimark R L, St John T, Suzuki S. (1994) Cloning of five human cadherins clarifies characteristic features of cadherin extracellular domain and provides further evidence for two structurally different types of cadherin. Cell Adhes Commun. 2(1) :15-26.
66. Suzuki S, Sano K, Tanihara H. (1991) Diversity of the cadherin family: evidence for eight new cadherins in nervous tissue. Cell Regul. 2(4):261-70.

1-23. (canceled)

24. A method of treating schizophrenia and/or affective psychosis in a subject, comprising administering to the subject a polynucleotide fragment or fragments comprising SEMCAP3, N33, GRIK4, NPAS3, PDE4B and/or CDH8 gene(s) or a fragment(s), derivative(s) or homologue(s) thereof.

25. The method according to claim 24 wherein the SEMCAP3 nucleotide fragment comprises the sequence found in the public database under accession number AF127084-AF127088, KIAA1095, AB029018, XM_{—}041363 or BC014432 or the sequence shown in FIG. 3.

26. The method according to claim 24 wherein the N33 polynucleotide fragment comprises the sequence found in the public database under accession number U42349 or BAC RP11-23;14 or the sequences shown in FIGS. 6 or 7.

27. The method according to claim 24 wherein the GRIK4 polynucleotide fragment comprises the sequence found in the public database under accession number NM_{—}014619 or the sequences shown in FIGS. 10 or 16.

28. The method according to claim 24 wherein the NPAS3 polynucleotide fragment comprises the sequence found in the public database under accession number AB054575 or AF164438 or the sequences shown in FIGS. 18 or 20.

29. The method according to claim 24 wherein the PDE4B polynucleotide fragment comprises the sequence as shown in FIGS. 25, 27 or 29.

30. The method according to claim 24 wherein the CDH8 polynucleotide comprises the sequencc found in the public database under accession number L34060, AB035305, NM_{—}001796, AB010436, AB010437, BAC CTC-420A11 or AC040161 or as shown in FIG. 35.

31. A method of treating schizophrenia and/or affective psychosis in a subject, comprising administering to the subject a polypeptide fragment or fragments comprising SEMCAP3, N33, GRIK4, NPAS3, PDE4B and/or CDH8 gene(s) or a fragment(s), derivative(s) or homologue(s) thereof.

32. The method according to claim 31 wherein the SEMCAP3 polypeptide fragment comprises the sequence found in the public database under accession number AAF22131, AAF22132 or XP_{—}041363, or as shown in FIG. 4.

33. The method according to claim 31 wherein the N33 polypeptide fragment comprises the sequence found in the public database under accession number Q13454 or as shown in FIGS. 6 or 7.

34. The method according to claim 31 wherein the GRIK4 polypeptide fragment comprises the sequence found in the public database under accession number NM_{—}014619, or as shown in FIGS. 11 and 17.

35. The method according to claim 31 wherein the PDE4B polypeptide fragment comprises the sequence as shown in FIGS. 26, 28 or 30.

36. The method according to claim 31 wherein the CDH8 polypeptide fragment comprises the sequence found in the public database under accession number NP_{—}001787 or as shown in FIG. 36.

37. The method according to claim 31 wherein the polynucleotide fragment or polypeptide fragment consists essentially of the identified sequences.

38. A method of diagnosing schizophrenia and/or affective psychosis or susceptibility to schizophrenia and/or affective psychosis in an individual, wherein the method comprises determining if SEMCAP3, N33, GRIK4, NPAS3, PDE4B and/or CDH8 gene(s) in the individual has/have been disrupted by a mutation or chromosomal rearrangement.

39. The method according to claim 38 wherein any disruption is determined by detecting a relative level of mRNA expressed by the/said SEMCAP3, N33, GRIK4, NPAS3, PDE4B and/or CDH8 gene(s).

40. The method according to claim 38 wherein a level of the/said SEMCAP3, N33, GRIK4, NPAS3, PDE4B and/or CDH8 gene products are detected by an immunological technique.

41. The method according to claim 40 wherein an antibody or antibodies specific for the/said gene(s) is used to detect said gene product(s).

42. A method for the treatment of schizophrenia and/or affective psychosis, comprising administering an antibody or antibodies specific for SEMCAP3, N33, GRIK4, NPAS2, PDE4B and/or CDH8.

43. An animal model for psychiatric disorders wherein the animal model has been generated by specifically disrupting expression of the/said SEMCAP3, N33, GRIK4, NPAS2, PDE4B and/or CDH8 gene(s).

44. An animal model for psychiatric disorder wherein the animal model has been generated by specifically upregulating expression of the/said SEMCAP3, N33, GRIK4, NPAS2, PDE4B and/or CDH8 gene(s).

45. A method for identifying ligands for SEMCAP3, N33, GRIK4, NPAS2, PDE4B and/or CDH8 gene(s) products, said method comprising the steps of:
a) introducing into a suitable host cell a polynucleotide fragment according to the invention;
b) culturing cells under conditions to allow expression of the polynucleotide fragment;
c) optionally isolating the expression product;
d) brining the expression product (or the host cell from step b)) into contact with potential ligands which will possibly bind to the protein encoded by said polynucleotide fragment from step a);
e) establishing whether a ligand has bound to the expressed protein; and
f) optionally isolating and identifying the ligand.


SEQUENCE LISTING (94 sequences)

SEQ ID NO: 1 - DNA, 4773 bp, Homo sapiens
aaaacttccccgggtagattcacccaccggtcctggaaacctgctaaatcctgaaggttcacagaacctctggtcagaactgaagttgcagccggagcttcccgcaggctctgtaactttccctggaatgaaataaataaataaagaccgtaagtgctgagatagcgggccccaagatatttttagtcctctgcaatcagccactagaggaagggggagggagaagggagtaaaaaagttttgatccgttcgggaaggggctcgaagagaacccttgggagaaagcagtagcctcagctccaaactcagcgagcttttctcggctggcgttttgtctcctatagcgtagactgtaagagaacagaaaggagtttcccgagaagattcaggctggcgtcctgggctggcccgtcccttctggcgagcctcagtgtcctcccacgcgcttctgccttccagcctcctccctttttcggggggctggcgggaggcatccaaggcacgatgtatgtgcgctcgcgctcgcgcaaatacggccggaggagtcctgttcctcgggcattttccgaggaagtctggatcaattaggctcagtccggggagagccagcgagcgcgcgggcggcgtagccggcctgtctgggccgcctcgtggggagggagggggcgcccggccgcccggcggcgaccccggggcctggccgccaccatgggcttcgagctggaccgcttcgacggcgacgtggacccggacctgaagtgcgcgctgtgccacaaggtcctggaggacccgctgaccacgccgtgcggccacgtcttctgcgccggctgcgtgctgccctgggtggtgcaggagggcagctgcccggcgcgctgccgcggtcgcctgtcggccaaagagctcaaccacgtcctgccgctcaagcgccttatcctcaagctggacatcaagtgcgcgtacgcgacgcgcggctgcggccgggtggtcaagctgcagcagctgccggagcacctcgagcgctgcgacttcgcgcccgcgcgctgtcgccacgcgggttgcggccaggtgctgctgcggcgcgacgtggaggcgcacatgcgcgacgcgtgcgacgcgcggccagtgggccgctgccaggagggctgcgggctacccttgacgcacggcgagcagcgcgcgggcggccactgctgcgcgcgagcgctgcgggcgcacaacggcgcgctccaggcccgcctgggcgcgctgcacaaggcgctcaagaaggaggcgctgcgcgctgggaagcgcgagaagtcgctggtggcccagctggccgcggcgcagcttgagctgcagatgaccgcgctgcgctaccagaagaaattcaccgaatacagcgcgcgcctcgactcgctcagccgctgcgtggccgcgccgcccggcggcaagggcgaagaaaccaaaagtctgactcttgtcctgcatcgggactccggctccctgggattcaatattattggtggccggccgagtgtggataaccacgatggatcatccagtgaaggaatctttgtatccaagatagttgacagtgggcctgcagccaaggaaggaggcctgcaaattcatgacaggattattgaggtcaacggcagagacttatccagagcaactcatgaccaggctgtggaagctttcaagacagccaaggagcccatagtggtgcaggtgttgagaagaacaccaaggaccaaaatgttcacgcctccatcagagtctcagctggtggacacgggaacccaaaccgacatcacctttgaacatatcatggccctcactaagatgtcctctcccagcccacccgtgctggatccctatctcttgccagaggagcatccctcagcccatgaatactacgatccaaatgactacattggagacatccatcaggagatggacagggaggagctggagctggaggaagtggacctctacagaatgaacagccaggacaagctgggcctcactgtgtgctaccggacggacgatgaagacgacattgggatttatatcagtgagattgaccctaacagcattgcagccaaggatgggcgcatccgagaaggagaccgcattatccagattaatgggatagaggtgcagaaccgtgaagaggctgtggctcttctaaccagtgaagaaaataaaaacttttcattgctgattgcaaggcctgaactccagctggatgagggctggatggatgatgacaggaacgactttctggatgacctgcacatggacatgctggaggagcagcaccaccaggccatgcaattcacagctagcgtgctgcagcagaagaagcacgacgaagacggtgggaccacagatacagccaccatcttgtccaaccagcacgagaaggacagcggtgtggggcggaccgacgagagcacccgtaatgacgagagctcggagcaagagaacaatggcgacgacgccaccgcatcctccaacccgctggcggggcagaggaagctcacctgcagccaggacaccttgggcagcggcgacctgcccttcagcaacgagtctttcatttcggccgactgcacggacgccgactacctggggatcccggtggacgagtgcgagcgcttccgcgagctcctggagctcaagtgccaggtgaagagcgccaccccttacggcctgtactaccctagcggccccctggacgccggcaagagtgaccctgagagcgtggacaaggagctggagctgctgaacgaagagctgcgcagcatcgagctggagtgcctgagcatcgtgcgcgcccacaagatgcagcagctcaaggagcagtaccgcgagtcctggatgctgcacaacagcggcttccgcaactacaacaccagcatcgacgtgcgcagacacgagctctcagatatcaccgagctcccggagaaatccgacaaggacagctcgagcgcctacaacacaggcgagagctgccgcagcaccccgctcaccctggagatctcccccgacaactccttgaggagagcggcggagggcatcagctgcccgagcagcgaaggggctgtggggaccacggaagcctacgggccagcctccaagaatctgctctccatcacggaagatcccgaagtgggcacccctacctatagcccgtccctgaaggagctggaccccaaccagcccctggaaagcaaagagcggagagccagcgacgggagccggagccccacgcccagccagaagctgggcagcgcctacctgccctcctatcaccactccccatacaagcacgcgcacatcccggcgcacgcccagcactaccagagctacatgcagctgatccagcagaagtcggccgtggagtacgcgcaaagccagatgagcctggtgagcatgtgcaaggacctgagctctcccaccccgtcggagccgcgcatggagtggaaggtgaagatccgcagcgacgggacgcgctacatcaccaagaggcccgtgcgggaccgcctgctgcgggagcgcgccctgaagatccgggaagagcgcagcggcatgaccaccgacgacgacgcggtgagcgagatgaagatggggcgctactggagcaaggaggagaggaagcagcacctggtgaaggccaaggagcagcggcggcggcgcgagttcatgatgcagagcaggttggattgtctcaaggagcagcaagcagccgatgacaggaaggagatgaacattctcgaactgagccacaaaaagatgatgaagaagaggaataagaaaatcttcgataactggatgacgatccaagaactcttaacccacggcacaaaatccccggacggcactagagtatacaattccttcctatcggtgactactgtataattttcacttctgcattatgtacataaaggagaccactaccactggggtagaaattcctgcctcgttcaatgcggcaagtttttgtatataagataagtacggtcttcatgtttatagtccaaatttgcaaaccctacaactctgggtgtcataggtctattttaagggaagagagagaaaaacacccttactatcttggaaggcaatattaacaaacagagcttttttcaaatagcaattgtacttttctacctgtacccttttacataaagtgtttaaatttcagaaagatcttttattaagcatactttcacagaataacttgtttaaactatattcatataaaaaagttaaacacgctttttttcctgcctaaaacacaaatacaactgccagtatgtatttttaatggaaccctattttataatggtacgttactgaatgtgtttcatatgcgtgaccgttaagatattatcatttaggtgaaggtttcaactcaaaaccacccaacccggtggttaacgatttaatacatataaccaaaccggcagcgtttagagttgggatatacatttaaacattttcctggttaaggttcccaagagagtgtaaaggttttagcagaaagcaaaatatcttgcatctttatggaagtttaaagcatgtttgcaaatattgcagcccattgaaagaatttgcatgtacaggaaagttgtggatggagacggtttgtggaattttaagtgctcattgtagtaaacttttgctttgtagatttgaaggtacagacttatacaggcaagttcacaaaatcatgattagttacaaacagtaaaatgaagttaaaataaattattattttct

SEQ ID NO: 2 - protein, 1066 aa, Homo sapiens
MGFELDRFDGDVDPDLKCALCHKVLEDPLTTPCGHVFCAGCVLPWVVQEGSCPARCRGRLSAKELNHVLPLKRLILKLDIKCAYATRGCGRVVKLQQLPEHLERCDFAPARCRHAGCGQVLLRRDVEAHMRDACDARPVGRCQEGCGLPLTHGEQRAGGHCCARALRAHNGALQARLGALHKALKKEALRAGKREKSLVAQLAAAQLELQMTALRYQKKFTEYSARLDSLSRCVAAPPGGKGEETKSLTLVLHRDSGSLGFNIIGGRPSVDNHDGSSSEGIFVSKIVDSGPAAKEGGLQIHDRIIEVNGRDLSRATHDQAVEAFKTAKEPIVVQVLRRTPRTKMFTPPSESQLVDTGTQTDITFEHIMALTKMSSPSPPVLDPYLLPEEHPSAHEYYDPNDYIGDIHQEMDREELELEEVDLYRMNSQDKLGLTVCYRTDDEDDIGIYISEIDPNSIAAKDGRIREGDRIIQINGIEVQNREEAVALLTSEENKNFSLLIARPELQLDEGWMDDDRNDFLDDLHMDMLEEQHHQAMQFTASVLQQKKHDEDGGTTDTATILSNQHEKDSGVGRTDESTRNDESSEQENNGDDATASSNPLAGQRKLTCSQDTLGSGDLPFSNESFISADCTDADYLGIPVDECERFRELLELKCQVKSATPYGLYYPSGPLDAGKSDPESVDKELELLNEELRSIELECLSIVRAHKMQQLKEQYRESWMLHNSGFRNYNTSIDVRRHELSDITELPEKSDKDSSSAYNTGESCRSTPLTLEISPDNSLRRAAEGISCPSSEGAVGTTEAYGPASKNLLSITEDPEVGTPTYSPSLKELDPNQPLESKERRASDGSRSPTPSQKLGSAYLPSYHHSPYKHAHIPAHAQHYQSYMQLIQQKSAVEYAQSQMSLVSMCKDLSSPTPSEPRMEWKVKIRSDGTRYITKRPVRDRLLRERALKIREERSGMTTDDDAVSEMKMGRYWSKEERKQHLVKAKEQRRRREFMMQSRLDCLKEQQAADDRKEMNILELSHKKMMKKRNKKIFDNWMTIQELLTHGTKSPDGTRVYNSFLSVTTV

SEQ ID NO: 3 - DNA, 115 bp, Homo sapiens
atcttcctcctgctctggctgtgtgaagatctgcctccttcctcttcggcttcatgcatgatcgtaagtttcctgaggcctcctcagccatgcttcctgcatagcctgcagaaat

SEQ ID NO: 4 - DNA, 277 bp, Homo sapiens
cccgggtccctcgcaaagccgctgccatcccggagggcccagccagcgggctcccggaggctggccgggcaggcgtggtgcgcggtaggagctgggcgcgcacggctaccgcgcgtggaggagacactgccctgccgcgatgggggcccggggcgctccttcacgccgtaggcaagcggggcggcggctgcggtacctgcccaccgggagctttcccttccttctcctgctgctgctgctctgcatccagctcgggggaggacagaagaaaaaggag

SEQ ID NO: 5 - DNA, 660 bp, Homo sapiens
aatcttttagctgaaaaagtagagcagctgatggaatggagttccagacgctcaatcttccgaatgaatggtgataaattccgaaaatttataaaggcaccacctcgaaactattccatgattgttatgttcactgctcttcagcctcagcggcagtgttctgtgtgcaggcaagctaatgaagaatatcaaatactggcgaactcctggcgctattcatctgctttttgtaacaagctcttcttcagtatggtggactatgatgaggggacagacgtttttcagcagctcaacatgaactctgctcctacattcatgcattttcctccaaaaggcagacctaagagagctgatacttttgacctccaaagaattggatttgcagctgagcaactagcaaagtggattgctgacagaacggatgttcatattcgggttttcagaccacccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtg

SEQ ID NO: 6 - DNA, 64 bp, Homo sapiens
agctacattcatgggagcagccaggctcagtttgtggcagaatcacacattattctggtactga

SEQ ID NO: 7 - DNA, 75 bp, Homo sapiens
atgccgctatcaccatggggatggttcttctaaatgaagcagcaacttcgaaaggcgatgttggaaaaagacgga

SEQ ID NO: 8 - DNA, 167 bp, Homo sapiens
tttaaccattctggaacattgtgttcagagccagaaaaattaatagattttattcacatctatgtctacggcttccttgacaactactgcagatgccgctatcaccatggggatggttcttctaaatgaagcagcaacttcgaaaggcgatgttggaaaaagacgga

SEQ ID NO: 9 - DNA, 91 bp, Homo sapiens
taatttgcctagtgggattgggcctggtggtcttcttcttcagttttctactttcaatatttcgttccaagtaccacggctatccttatag

SEQ ID NO: 10 - DNA, 65 bp, Homo sapiens
tgatctggactttgagtgagaagatgtgatttggaccatggcacttaaaaactctataacctcag

SEQ ID NO: 11 - DNA, 92 bp, Homo sapiens
ctttttaattaaatgaagccaagtgggatttgcataaagtgaatgtttaccatgaagataaactgttcctgactttatactattttgaattc

SEQ ID NO: 12 - protein, 29 aa, Homo sapiens
MEWSSRRSIFRMNGDKFRKFIKAPPRNYS

SEQ ID NO: 13 - protein, 85 aa, Homo sapiens
MGARGAPSRRRQAGRRLRYLPTGSFPFLLLLLLLCIQLGGGQKKKENLLAEKVEQLMEWSSRRSIFRMNGDKFRKFIKAPPRNYS

SEQ ID NO: 14 - DNA, 1047 bp, Homo sapiens
aatcttttagctgaaaaagtagagcagctgatggaatggagttccagacgctcaatcttccgaatgaatggtgataaattccgaaaatttataaaggcaccacctcgaaactattccatgattgttatgttcactgctcttcagcctcagcggcagtgttctgtgtgcaggcaagctaatgaagaatatcaaatactggcgaactcctggcgctattcatctgctttttgtaacaagctcttcttcagtatggtggactatgatgaggggacagacgtttttcagcagctcaacatgaactctgctcctacattcatgcattttcctccaaaaggcagacctaagagagctgatacttttgacctccaaagaattggatttgcagctgagcaactagcaaagtggattgctgacagaacggatgttcatattcgggttttcagaccacccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtgagctacattcatgggagcagccaggctcagtttgtggcagaatcacacattattctggtactgaatgccgctatcaccatggggatggttcttctaaatgaagcagcaacttcgaaaggcgatgttggaaaaagacggataatttgcctagtgggattgggcctggtggtcttcttcttcagttttctactttcaatatttcgttccaagtaccacggctatccttatagtgatctggactttgagtgagaagatgtgatttggaccatggcacttaaaaactctataacctcagctttttaattaaatgaagccaagtgggatttgcataaagtgaatgtttaccatgaagataaactgttcctgactttatactattttgaattc

SEQ ID NO: 15 - protein, 348 aa, Homo sapiens
MGARGAPSRRRQAGRRLRYLPTGSFPFLLLLLLLCIQLGGGQKKKENLLAEKVEQLMEWSSRRSIFRMNGDKFRKFIKAPPRNYSMIVMFTALQPQRQCSVCRQANEEYQILANSWRYSSAFCNKLFFSMVDYDEGTDVFQQLNMNSAPTFMHFPPKGRPKRADTFDLQRIGFAAEQLAKWIADRTDVHIRVFRPPNYSGTIALALLVSLVGGLLYLRRNNLEFIYNKTGWAMVSLCIVFAMTSGQMWNHIRGPPYAHKNPHNGQVSYIHGSSQAQFVAESHIILVLNAAITMGMVLLNEAATSKGDVGKRRIICLVGLGLVVFFFSFLLSIFRSKYHGYPYSDLDFE

SEQ ID NO: 16 - DNA, 982 bp, Homo sapiens
aatcttttagctgaaaaagtagagcagctgatggaatggagttccagacgctcaatcttccgaatgaatggtgataaattccgaaaatttataaaggcaccacctcgaaactattccatgattgttatgttcactgctcttcagcctcagcggcagtgttctgtgtgcaggcaagctaatgaagaatatcaaatactggcgaactcctggcgctattcatctgctttttgtaacaagctcttcttcagtatggtggactatgatgaggggacagacgtttttcagcagctcaacatgaactctgctcctacattcatgcattttcctccaaaaggcagacctaagagagctgatacttttgacctccaaagaattggatttgcagctgagcaactagcaaagtggattgctgacagaacggatgttcatattcgggttttcagaccacccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtgagctacattcatgggagcagccaggctcagtttgtggcagaatcacacattattctggtactgaatgccgctatcaccatggggatggttcttctaaatgaagcagcaacttcgaaaggcgatgttggaaaaagacggataatttgcctagtgggattgggcctggtggtcttcttcttcagttttctactttcaatatttcgttccaagtaccacggctatccttatagctttttaattaaatgaagccaagtgggatttgcataaagtgaatgtttaccatgaagataaactgttcctgactttatactattttgaattc

SEQ ID NO: 17 - protein, 347 aa, Homo sapiens
MGARGAPSRRRQAGRRLRYLPTGSFPFLLLLLLLCIQLGGGQKKKENLLAEKVEQLMEWSSRRSIFRMNGDKFRKFIKAPPRNYSMIVMFTALQPQRQCSVCRQANEEYQILANSWRYSSAFCNKLFFSMVDYDEGTDVFQQLNMNSAPTFMHFPPKGRPKRADTFDLQRIGFAAEQLAKWIADRTDVHIRVFRPPNYSGTIALALLVSLVGGLLYLRRNNLEFIYNKTGWAMVSLCIVFAMTSGQMWNHIRGPPYAHKNPHNGQVSYIHGSSQAQFVAESHIILVLNAAITMGMVLLNEAATSKGDVGKRRIICLVGLGLVVFFFSFLLSIFRSKYHGYPYSFLIK

SEQ ID NO: 18 - DNA, 752 bp, Homo sapiens
aatcttttagctgaaaaagtagagcagctgatggaatggagttccagacgctcaatcttccgaatgaatggtgataaattccgaaaatttataaaggcaccacctcgaaactattccatgattgttatgttcactgctcttcagcctcagcggcagtgttctgtgtgcaggcaagctaatgaagaatatcaaatactggcgaactcctggcgctattcatctgctttttgtaacaagctcttcttcagtatggtggactatgatgaggggacagacgtttttcagcagctcaacatgaactctgctcctacattcatgcattttcctccaaaaggcagacctaagagagctgatacttttgacctccaaagaattggatttgcagctgagcaactagcaaagtggattgctgacagaacggatgttcatattcgggttttcagaccacccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtgctttttaattaaatgaagccaagtgggatttgcataaagtgaatgtttaccatgaagataaactgttcctgactttatactattttgaattc

SEQ ID NO: 19 - protein, 269 aa, Homo sapiens
MGARGAPSRRRQAGRRLRYLPTGSFPFLLLLLLLCIQLGGGQKKKENLLAEKVEQLMEWSSRRSIFRMNGDKFRKFIKAPPRNYSMIVMFTALQPQRQCSVCRQANEEYQILANSWRYSSAFCNKLFFSMVDYDEGTDVFQQLNMNSAPTFMHFPPKGRPKRADTFDLQRIGFAAEQLAKWIADRTDVHIRVFRPPNYSGTIALALLVSLVGGLLYLRRNNLEFIYNKTGWAMVSLCIVFAMTSGQMWNHIRGPPYAHKNPHNGQVLFN

SEQ ID NO: 20 - DNA, 891 bp, Homo sapiens
aatcttttagctgaaaaagtagagcagctgatggaatggagttccagacgctcaatcttccgaatgaatggtgataaattccgaaaatttataaaggcaccacctcgaaactattccatgattgttatgttcactgctcttcagcctcagcggcagtgttctgtgtgcaggcaagctaatgaagaatatcaaatactggcgaactcctggcgctattcatctgctttttgtaacaagctcttcttcagtatggtggactatgatgaggggacagacgtttttcagcagctcaacatgaactctgctcctacattcatgcattttcctccaaaaggcagacctaagagagctgatacttttgacctccaaagaattggatttgcagctgagcaactagcaaagtggattgctgacagaacggatgttcatattcgggttttcagaccacccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtgagctacattcatgggagcagccaggctcagtttgtggcagaatcacacattattctggtactgaatgccgctatcaccatggggatggttcttctaaatgaagcagcaacttcgaaaggcgatgttggaaaaagacggactttttaattaaatgaagccaagtgggatttgcataaagtgaatgtttaccatgaagataaactgttcctgactttatactattttgaattc

SEQ ID NO: 21 - protein, 314 aa, Homo sapiens
MGARGAPSRRRQAGRRLRYLPTGSFPFLLLLLLLCIQLGGGQKKKENLLAEKVEQLMEWSSRRSIFRMNGDKFRKFIKAPPRNYSMIVMFTALQPQRQCSVCRQANEEYQILANSWRYSSAFCNKLFFSMVDYDEGTDVFQQLNMNSAPTFMHFPPKGRPKRADTFDLQRIGFAAEQLAKWIADRTDVHIRVFRPPNYSGTIALALLVSLVGGLLYLRRNNLEFIYNKTGWAMVSLCIVFAMTSGQMWNHIRGPPYAHKNPHNGQVSYIHGSSQAQFVAESHIILVLNAAITMGMVLLNEAATSKGDVGKRRTF

SEQ ID NO: 22 - DNA, 1010 bp, Homo sapiens
aatcttttagctgaaaaagtagagcagctgatggaatggagttccagacgctcaatcttccgaatgaatggtgataaattccgaaaatttataaaggcaccacctcgaaactattccatgattgttatgttcactgctcttcagcctcagcggcagtgttctgtgtgcaggcaagctaatgaagaatatcaaatactggcgaactcctggcgctattcatctgctttttgtaacaagctcttcttcagtatggtggactatgatgaggggacagacgtttttcagcagctcaacatgaactctgctcctacattcatgcattttcctccaaaaggcagacctaagagagctgatacttttgacctccaaagaattggatttgcagctgagcaactagcaaagtggattgctgacagaacggatgttcatattcgggttttcagaccacccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtgtttaaccattctggaacattgtgttcagagccagaaaaattaatagattttattcacatctatgtctacggcttccttgacaactactgcagatgccgctatcaccatggggatggttcttctaaatgaagcagcaacttcgaaaggcgatgttggaaaaagacggataatttgcctagtgggattgggcctggtggtcttcttcttcagttttctactttcaatatttcgttccaagtaccacggctatccttatagctttttaattaaatgaagccaagtgggatttgcataaagtgaatgtttaccatgaagataaactgttcctgactttatactattttgaattc

SEQ ID NO: 23 - protein, 308 aa, Homo sapiens
MGARGAPSRRRQAGRRLRYLPTGSFPFLLLLLLLCIQLGGGQKKKENLLAEKVEQLMEWSSRRSIFRMNGDKFRKFIKAPPRNYSMIVMFTALQPQRQCSVCRQANEEYQILANSWRYSSAFCNKLFFSMVDYDEGTDVFQQLNMNSAPTFMHFPPKGRPKRADTFDLQRIGFAAEQLAKWIADRTDVHIRVFRPPNYSGTIALALLVSLVGGLLYLRRNNLEFIYNKTGWAMVSLCIVFAMTSGQMWNHIRGPPYAHKNPHNGQVFNHSGTLCSEPEKLIDFIHIYVYGFLDNYCRCRYHHGDGSSK

SEQ ID NO: 24 - DNA, 919 bp, Homo sapiens
aatcttttagctgaaaaagtagagcagctgatggaatggagttccagacgctcaatcttccgaatgaatggtgataaattccgaaaatttataaaggcaccacctcgaaactattccatgattgttatgttcactgctcttcagcctcagcggcagtgttctgtgtgcaggcaagctaatgaagaatatcaaatactggcgaactcctggcgctattcatctgctttttgtaacaagctcttcttcagtatggtggactatgatgaggggacagacgtttttcagcagctcaacatgaactctgctcctacattcatgcattttcctccaaaaggcagacctaagagagctgatacttttgacctccaaagaattggatttgcagctgagcaactagcaaagtggattgctgacagaacggatgttcatattcgggttttcagaccacccaactactctggtaccattgctttggccctgttagtgtcgcttgttggaggtttgctttatttgagaaggaacaacttggagttcatctataacaagactggttgggccatggtgtctctgtgtatagtctttgctatgacttctggccagatgtggaaccatatccgtggacctccatatgctcataagaacccacacaatggacaagtgtttaaccattctggaacattgtgttcagagccagaaaaattaatagattttattcacatctatgtctacggcttccttgacaactactgcagatgccgctatcaccatggggatggttcttctaaatgaagcagcaacttcgaaaggcgatgttggaaaaagacggactttttaattaaatgaagccaagtgggatttgcataaagtgaatgtttaccatgaagataaactgttcctgactttatactattttgaattc

SEQ ID NO: 25 - protein, 308 aa, Homo sapiens; identical to SEQ ID NO: 23

SEQ ID NO: 26 - protein, 335 aa, Homo sapiens
MAARWRFWCVSVTMVVALLIVCDVPSASAQRKKEMVLSEKVSQLMEWTNKRPVIRMNGDKFRRLVKAPPRNYSVIVMFTALQLHRQCVVCKQADEEFQILANSWRYSSAFTNRIFFAMVDFDEGSDVFQMLNMNSAPTFINFPAKGKPKRGDTYELQVRGFSAEQIARWIADRTDVNIRVIRPPNYAGPLMLGLLLAVIGGLVYLRRSNMEFLFNKTGWAFAALCFVLAMTSGQMWNHIRGPPYAHKNPHTGHVNYIHGSSQAQFVAETHIVLLFNGGVTLGMVLLCEAATSDMDIGKRKIMCVAGIGLVVLFFSWMLSIFRSKYHGYPYSFLMS

SEQ ID NO: 27 - protein, 348 aa, Homo sapiens; identical to SEQ ID NO: 15

SEQ ID NO: 28 - protein, 347 aa, Homo sapiens; identical to SEQ ID NO: 17

SEQ ID NO: 29 - protein, 350 aa, Homo sapiens
MGARGAPSRRRQAGRRLRYLPTGSFPFLLLLLLLCIQLGGGQKKKENLLAEKVEQLMEWSSRRSIFRMNGDKFRKFIKAPPRNYSMIVMFTALQPQRQCSVCRQANEEYQILANSWRYSSAFCNKLFFSMVDYDEGTDVFQQLNMNSAPTFMHFPPKGRPKRADTFDLQRIGFAAEQLAKWIADRTDVHIRVFRPPNYSGTIALALLVSLVGGLLYLRRNNLEFIYNKTGWAMVSLCIVFAMTSGQMWNHIRGPPYAHKNPHNGQVSYIHGSSQAQFVAESHIILVLNAAITMGMVLLNEAATSKGDVGKRRTFIICLVGLGLVVFFFSFLLSIFRSKYHGYPYSDLDFE

SEQ ID NO: 30 - protein, 347 aa, Drosophila melanogaster
MRLLHKTLLSGLLVVALFAIYAAAQSKSKTGLSLSEKVQNLVDMNAKKPLLRFNGPKFREYVKSAPRNYSMIVMLTALAPSRQCQICRHAHDEFAIVANSYRFSSTYSNKLFFAMVDFDDGSEVFQLLRLNTAPVFMHFPAKGKPKGADTMDIHRVGFAADSIAKFVAERTDITIRIFRPPNYSGTVAMITLVALVGSFLYIRRNNLEFLYNKNLWGAIAVFFCFAMISGQMWNHIRGPPLVHKSQNGGVAYIHGSSQGQLVVETYIVMFLNAMIVLGMILLIESGTPKAHNKNRIMAMTGLVLLTVFFSFLLSVFRSKAQGYPYISCSNRIDCSPVPVQVHPISFL

SEQ ID NO: 31 - protein, 331 aa, Caenorhabditis elegans
MLLAVYESAQQQTLEDKVQNLVDLTSRQSIVKFNMDKWKTLVRMQPRNYSMIVMFTALSPGVQCPICKPAYDEFMIVANSHRYTSSEGDRRKVFFGIVDYEDAPQIFQQMNLNTAPILYHFGPKLGAKKRPEQMDFQRQGFDADAIGRFVADQTEVHVRVIRPPNYTAPVVIALFVALLLGMLYMKRNSLDFLFNRTVWGFVCLAITFIFMSGQMWNHIRGPPFMITNPNTKEPSFIHGSTQFQLIAETYIVGLLYALIAIGFICVNEAADQSNSKDRKNAGKKLNPLSLLNIPTNTLAIAGLVCICVFFSFLLSVFRSKYRGYPYSFLFA

SEQ ID NO: 32 - protein, 350 aa, Saccharomyces cerevisiae
MNWLFLVSLVFFCGVSTHPALAMSSNRLLKLANKSPKKIIPLKDSSFENILAPPHENAYIVALFTATAPEIGCSLCLELESEYDTIVASWFDDHPDAKSSNSDTSIFFTKVNLEDPSKTIPKAFQFFQLNNVPRLFIFKPNSPSILDHSVISISTDTGSERMKQIIQAIKQFSQVNDFSLHLPMDWTPIITSTIITFITVLLFKKQSKLMFSIISSRIIWATLSTFFIICMISAYMFNQIRNTQLAGVGPKGEVMYFLPNEFQHQFAIETQVMVLIYGTLAALVVVLVKGIQFLRSHLYPETKKAYFIDAILASFCALFIYVFFAALTTVFTIKSPAYPFPLLRLSAPFK

SEQ ID NO: 33 - protein, 332 aa, Saccharomyces cerevisiae
MKWCSTYIIIWLAIIFHKFQKSTATASHNIDDILQLKDDTGVITVTADNYPLLSRGVPGYFNILYITMRGTNSNGMSCQLCHDFEKTYHAVADVIRSQAPQSLNLFFTVDVNEVPQLVKDLKLQNVPHLVVYPPAESNKQSQFEWKTSPFYQYSLVPENAENTLQFGDFLAKILNISITVPQAFNVQEFVYYFVACMVVFIFIKKVILPKVTNKWKLFSMILSLGILLPSITGYKFVEMNAIPFIARDAKNRIMYFSGGSGWQFGIEIFSVSLMYIVMSALSVLLIYVPKISCVSEKMRGLLSSFLACVLFYFFSYFISCYLIKNPGYPIVF

SEQ ID NO: 34 - protein, 108 aa, Homo sapiens
LVSLVGGLLYLRRNNLEFIYNKTGWAMVSLCIVFAMTSGQMWNHIRGPPYAHKNPHNGQVSYIHGSSQAQFVAESHIILVLNAAITMGMVLLNEAATSKGDVGKRRTF

SEQ ID NO: 35 - protein, 142 aa, Homo sapiens
LVSLVGGLLYLRRNNLEFIYNKTGWAMVSLCIVFAMTSGQMWNHIRGPPYAHKNPHNGQVSYIHGSSQAQFVAESHIILVLNAAITMGMVLLNEAATSKGDVGKRRIICLVGLGLVVFFFSFLLSIFRSKYHGYPYSDLDFE

SEQ ID NO: 36 - protein, 141 aa, Homo sapiens
LVSLVGGLLYLRRNNLEFIYNKTGWAMVSLCIVFAMTSGQMWNHIRGPPYAHKNPHNGQVSYIHGSSQAQFVAESHIILVLNAAITMGMVLLNEAATSKGDVGKRRIICLVGLGLVVFFFSFLLSIFRSKYHGYPYSFLIK

SEQ ID NO: 37 - protein, 63 aa, Homo sapiens
LVSLVGGLLYLRRNNLEFIYNKTGWAMVSLCIVFAMTSGQMWNHIRGPPYAHKNPHNGQVLFN

SEQ ID NO: 38 - protein, 102 aa, Homo sapiens
LVSLVGGLLYLRRNNLEFIYNKTGWAMVSLCIVFAMTSGQMWNHIRGPPYAHKNPHNGQVFNHSGTLCSEPEKLIDFIHIYVYGFLDNYCRCRYHHGDGSSK

SEQ ID NO: 39 - protein, 102 aa, Homo sapiens; identical to SEQ ID NO: 38

SEQ ID NO: 40 - DNA, 2871 bp, Homo sapiens
atgccccgcgtctcggcgcctttggtgctgcttcctgcgtggctcgtgatggtcgcctgcagcccgcactccttgaggatcgctgctatcttggacgaccccatggagtgcagcagaggggagcggctctccatcaccctggccaagaaccgcatcaaccgcgctcctgagaggctgggcaaggccaaggtcgaagtggacatctttgagcttctcagagacagcgagtacgagactgcagaaaccatgtgtcagatcctccccaagggggtggtcgctgtcctcggaccatcgtccagcccagcctccagctccatcatcagcaacatctgtggagagaaggaggtccctcacttcaaagtggccccagaggagttcgtcaagttccagttccagagattcacaaccctgaacctccaccccagcaacactgacatcagcgtggctgtagctgggatcctgaacttcttcaactgcaccaccgcctgcctcatctgtgccaaagcagaatgccttttaaacctagagaagctgctccggcaattccttatctccaaggacacgctgtccgtccgcatgctggatgacacccgggaccccaccccgctcctcaaggagatccgggacgacaagaccgccaccatcatcatccacgccaacgcctccatgtcccacaccatcctcctgaaggcagccgaacttgggatggtgtcagcctattacacatacatcttcactaatctggagttctcactccagagaacggacagccttgtggatgatcgtgtcaacatcctgggattttccattttcaaccaatcccatgctttcttccaagagtttgcccagagcctcaaccagtcctggcaggagaactgtgaccatgtgcccttcactgggcctgcgctctcctcggccctgctgtttgatgctgtctatgctgtggtgactgcggtgcaggaactgaaccggagccaagagatcggcgtgaagcccttgtcctgcggctcggcccagatctggcagcacggcaccagcctcatgaactacctgcgcatggtagaattggaaggtcttaccggccacattgaattcaacagcaaaggccagaggtccaactacgctttgaaaatcttacagttcacaaggaatggttttcggcagatcggccagtggcacgtggcagagggcctcagcatggacagccacctctatgcctccaacatctcggacactctcttcaacaccaccctggtcgtcaccaccatcctggaaaacccatatttaatgctgaaggggaaccaccaggagatggaaggcaatgaccgctacgagggcttctgtgtggacatgctcaaggagctggcagagatcctccgattcaactacaagatccgcctggttggggatggcgtgtacggcgttcccgaggccaacggcacctggacgggaatggtcggggagctgatcgctaggaaagcagatctggctgtggcaggcctcaccattacagctgaacgggagaaggtgattgatttctctaagccattcatgactctgggaattagcattctttaccgcattcatatgggacgcaaacccggctatttctccttcctggacccattttctccgggcgtctggctcttcatgcttctagcctatctggccgtcagctgtgtcctcttcctggtggctcggttgacgccctacgagtggtacagcccacacccatgtgcccagggccggtgcaacctcctggtgaaccagtactccctgggcaacagcctctggtttccggtcggggggttcatgcagcagggctccaccatcgcccctcgcgccttatccacccgctgtgtcagtggcgtctggtgggcattcacgctgatcatcatctcatcctacacggccaacctggcagccttcctgaccgtgcagcgcatggatgtgcccattgagtcagtggatgacctggctgaccagaccgccattgaatatggcacaattcacggaggctccagcatgaccttcttccaaaattcccgctaccagacctaccaacgcatgtggaattacatgtattccaagcagcccagcgtgttcgtgaagagcacagaggagggaatcgccagggtgttgaattccaactacgccttcctcctggaatccaccatgaacgagtactatcggcagcgaaactgcaacctcactcagattgggggcctgctggacaccaagggctatgggattggcatgccagtcggctcggttttccgggacgagtttgatctggccattctccagctgcaggagaacaaccgcctggagatcctgaagcgcaaatggtgggaaggagggaagtgccccaaggaggaagatcacagagctaaaggcctgggaatggagaatattggtggaatctttgtggttcttatttgtggcttaatcgtggccatttttatggctatgttggagtttttatggactctcagacactcagaagcaactgaggtgtccgtctgccaggagatggtgaccgagctgcgcagcattatcctgtgtcaggacagtatccacccccgccggcggcgcgccgcagtcccgccgccccggccccccatccccgaggagcgccgaccgcggggcacggcgacgctcagcaacgggaagctgtgcggggcaggggagcccgaccagctcgcgcagagactggcgcaggaggccgccctggtggcccgcggctgcacgcacatccgcgtctgccccgagtgccgccgcttccagggcctgcgggcacggccgtcgcccgcccgcagcgaggagagcctggagtgggagaaaaccaccaacagcagcgagcccgagtag

SEQ ID NO: 41 - protein, 956 aa, Homo sapiens [sequence omitted]

SEQ ID NO: 42 - DNA, 100 bp, Homo sapiens
gcgtggtagcatgtgcctgtaatcccagtgctttgggacaccgaggcaggaggatcactcgagcccaggagtgcgaggctgcagtgagttatgatcatac

SEQ ID NO: 43 - DNA, 212 bp, Homo sapiens
agatttgtcttctctgccaggtgacgctagacttcaggaagaccccccatttctgctccactcctgggcttggagaagagtacagctgctcttgactggtgggaccttttgctggctaggggtgatgggagaagcaagagagggatccacacacctgcgcttagctttctatgacctgggcggatggaggccaaaggtaaggtgggatgaga

SEQ ID NO: 44 - protein, 5 aa, Homo sapiens
MEAKA

SEQ ID NO: 45 - DNA, 118 bp, Homo sapiens
ccatgaggattcatagaagatgccccgcgtctcggcgcctttggtgctgcttcctgcgtggctcgtgatggtcgcctgcagcccgcactccttgaggatcggtaagtgtggcccagct

SEQ ID NO: 46 - protein, 28 aa, Homo sapiens
MPRVSAPLVLLPAWLVMVACSPHSLRIA

SEQ ID NO: 47 - DNA, 193 bp, Homo sapiens
gaaaccccccccagctgctatcttggacgaccccatggagtgcagcagaggggagcggctctccatcaccctggccaagaaccgcatcaaccgcgctcctgagaggctgggcaaggccaaggtcgaagtggacatctttgagcttctcagagacagcgagtacgagactgcagaaaccagtacgtagactggg

SEQ ID NO: 48 - protein, 55 aa, Homo sapiens
AILDDPMECSRGERLSITLAKNRINRAPERLGKAKVEVDIFELLRDSEYETAETM

SEQ ID NO: 49 - DNA, 3048 bp, Homo sapiens
gcgtggtagcatgtgcctgtaatcccagtgctttgggacaccgaggcaggaggatcactcgagcccaggagtgcgaggctgcagtgacgctagacttcaggaagaccccccatttctgctccactcctgggcttggagaagagtacagctgctcttgactggtgggaccttttgctggctaggggtgatgggagaagcaagagagggatccacacacctgcgcttagctttctatgacctgggcggatggaggccaaagctgctatcttggacgaccccatggagtgcagcagaggggagcggctctccatcaccctggccaagaaccgcatcaaccgcgctcctgagaggctgggcaaggccaaggtcgaagtggacatctttgagcttctcagagacagcgagtacgagactgcagaaaccatgtgtcagatcctccccaagggggtggtcgctgtcctcggaccatcgtccagcccagcctccagctccatcatcagcaacatctgtggagagaaggaggtccctcacttcaaagtggccccagaggagttcgtcaagttccagttccagagattcacaaccctgaacctccaccccagcaacactgacatcagcgtggctgtagctgggatcctgaacttcttcaactgcaccaccgcctgcctcatctgtgccaaagcagaatgccttttaaacctagagaagctgctccggcaattccttatctccaaggacacgctgtccgtccgcatgctggatgacacccgggaccccaccccgctcctcaaggagatccgggacgacaagaccgccaccatcatcatccacgccaacgcctccatgtcccacaccatcctcctgaaggcagccgaacttgggatggtgtcagcctattacacatacatcttcactaatctggagttctcactccagagaacggacagccttgtggatgatcgtgtcaacatcctgggattttccattttcaaccaatcccatgctttcttccaagagtttgcccagagcctcaaccagtcctggcaggagaactgtgaccatgtgcccttcactgggcctgcgctctcctcggccctgctgtttgatgctgtctatgctgtggtgactgcggtgcaggaactgaaccggagccaagagatcggcgtgaagcccttgtcctgcggctcggcccagatctggcagcacggcaccagcctcatgaactacctgcgcatggtagaattggaaggtcttaccggccacattgaattcaacagcaaaggccagaggtccaactacgctttgaaaatcttacagttcacaaggaatggttttcggcagatcggccagtggcacgtggcagagggcctcagcatggacagccacctctatgcctccaacatctcggacactctcttcaacaccaccctggtcgtcaccaccatcctggaaaacccatatttaatgctgaaggggaaccaccaggagatggaaggcaatgaccgctacgagggcttctgtgtggacatgctcaaggagctggcagagatcctccgattcaactacaagatccgcctggttggggatggcgtgtacggcgttcccgaggccaacggcacctggacgggaatggtcggggagctgatcgctaggaaagcagatctggctgtggcaggcctcaccattacagctgaacgggagaaggtgattgatttctctaagccattcatgactctgggaattagcattctttaccgcattcatatgggacgcaaacccggctatttctccttcctggacccattttctccgggcgtctggctcttcatgcttctagcctatctggccgtcagctgtgtcctcttcctggtggctcggttgacgccctacgagtggtacagcccacacccatgtgcccagggccggtgcaacctcctggtgaaccagtactccctgggcaacagcctctggtttccggtcggggggttcatgcagcagggctccaccatcgcccctcgcgccttatccacccgctgtgtcagtggcgtctggtgggcattcacgctgatcatcatctcatcctacacggccaacctggcagccttcctgaccgtgcagcgcatggatgtgcccattgagtcagtggatgacctggctgaccagaccgccattgaatatggcacaattcacggaggctccagcatgaccttcttccaaaattcccgctaccagacctaccaacgcatgtggaattacatgtattccaagcagcccagcgtgttcgtgaagagcacagaggagggaatcgccagggtgttgaattccaactacgccttcctcctggaatccaccatgaacgagtactatcggcagcgaaactgcaacctcactcagattgggggcctgctggacaccaagggctatgggattggcatgccagtcggctcggttttccgggacgagtttgatctggccattctccagctgcaggagaacaaccgcctggagatcctgaagcgcaaatggtgggaaggagggaagtgccccaaggaggaagatcacagagctaaaggcctgggaatggagaatattggtggaatctttgtggttcttatttgtggcttaatcgtggccatttttatggctatgttggagtttttatggactctcagacactcagaagcaactgaggtgtccgtctgccaggagatggtgaccgagctgcgcagcattatcctgtgtcaggacagtatccacccccgccggcggcgcgccgcagtcccgccgccccggccccccatccccgaggagcgccgaccgcggggcacggcgacgctcagcaacgggaagctgtgcggggcaggggagcccgaccagctcgcgcagagactggcgcaggaggccgccctggtggcccgcggctgcacgcacatccgcgtctgccccgagtgccgccgcttccagggcctgcgggcacggccgtcgcccgcccgcagcgaggagagcctggagtgggagaaaaccaccaacagcagcgagcccgagtag

SEQ ID NO: 50 - protein, 933 aa, Homo sapiens
MEAKAAILDDPMECSRGERLSITLAKNRINRAPERLGKAKVEVDIFELLRDSEYETAETMCQILPKGVVAVLGPSSSPASSSIISNICGEKEVPHFKVAPEEFVKFQFQRFTTLNLHPSNTDISVAVAGILNFFNCTTACLICAKAECLLNLEKLLRQFLISKDTLSVRMLDDTRDPTPLLKEIRDDKTATIIIHANASMSHTILLKAAELGMVSAYYTYIFTNLEFSLQRTDSLVDDRVNILGFSIFNQSHAFFQEFAQSLNQSWQENCDHVPFTGPALSSALLFDAVYAVVTAVQELNRSQEIGVKPLSCGSAQIWQHGTSLMNYLRMVELEGLTGHIEFNSKGQRSNYALKILQFTRNGFRQIGQWHVAEGLSMDSHLYASNISDTLFNTTLVVTTILENPYLMLKGNHQEMEGNDRYEGFCVDMLKELAEILRFNYKIRLVGDGVYGVPEANGTWTGMVGELIARKADLAVAGLTITAEREKVIDFSKPFMTLGISILYRIHMGRKPGYFSFLDPFSPGVWLFMLLAYLAVSCVLFLVARLTPYEWYSPHPCAQGRCNLLVNQYSLGNSLWFPVGGFMQQGSTIAPRALSTRCVSGVWWAFTLIIISSYTANLAAFLTVQRMDVPIESVDDLADQTAIEYGTIHGGSSMTFFQNSRYQTYQRMWNYMYSKQPSVFVKSTEEGIARVLNSNYAFLLESTMNEYYRQRNCNLTQIGGLLDTKGYGIGMPVGSVFRDEFDLAILQLQENNRLEILKRKWWEGGKCPKEEDHRAKGLGMENIGGIFVVLICGLIVAIFMAMLEFLWTLRHSEATEVSVCQEMVTELRSIILCQDSIHPRRRRAAVPPPRPPIPEERRPRGTATLSNGKLCGAGEPDQLAQRLAQEAALVARGCTHIRVCPECRRFQGLRARPSPARSEESLEWEKTTNSSEPE

SEQ ID NO: 51 - DNA, 3428 bp, Homo sapiens
ccacgcgtccgacgccccccacccgggaggggggagagaggcaaaaagtaagagaggaaaaaaaatagcaggaagatggcgcccaccaagcccagctttcagcaggatccttccaggcgagaacgtttacaagcattgagaaaggagaaatcccgagatgctgctcgctcccgccggggaaaagaaaactttgagttctatgaattggccaagttgttgcctcttcctgcagccattaccagccagctcgacaaggcatccatcattcgacttacaattagctatctgaaaatgagggactttgctaaccagggggaccctccgtggaacttgcgaatggaaggccctccacctaacacatcagtaaaaggtgcacagcgaaggagaagccccagtgcactagccattgaagtatttgaagcacatttgggaagccacattttgcagtccctggatggctttgtatttgcactaaatcaggaaggaaaatttttgtacatttccgaaacagtctccatctacctaggcctctcacaagtggagctgacaggcagcagtgtctttgactatgtccaccccggagatcacgtggagatggctgagcagctgggcatgaagctcccccctgggcggggtctcctgtcacagggcactgctgaggacggagccagctcagcatcttcctcctctcagtcggagacccccgagccagtggagtcaaccagccccagtctgctaaccactgacaacactcttgagcgttcctttttcatccgaatgaaatctactctgaccaaacgcggtgtgcacatcaaatcatcaggatataaggtgattcacataacaggccggctacgcctgagagtgtcgctgtcccacgggaggaccgtccccagccaaatcatgggtctcgtggttgttgcgcatgccttgcctccccctacgatcaatgaagtcagaattgactgccatatgttcgtcactcgagtaaatatggacctcaatatcatttactgtgaaaataggattagtgattatatggatctgacccctgtagatatcgtagggaagagatgctaccacttcatccatgctgaagacgtggagggcatcaggcacagtcacttggacttgctgaataagggtcagtgtgtgacaaagtactatcgctggatgcagaagaacggaggatatatttggatacagtccagtgccaccatagctattaatgccaagaatgcaaatgaaaagaatatcatctgggtgaattaccttcttagcaatcctgagtacaaggacacacccatggacatcgcacagctcccccatctgccggagaaaacttccgaatcctcggagacatccgactctgagtcagactctaaagacacctcaggtattacagaggacaacgagaactccaagtccgacgagaaggggaaccagtccgagaacagcgaagacccggagcccgaccggaagaagtcgggcaacgcgtgtgacaacgacatgaactgcaacgacgacggccacagctccagtaacccggacagccgcgacagcgacgacagcttcgagcactcggactttgagaaccccaaggcgggcgaggacggcttcggtgctctgggcgcgatgcagatcaaggtggagcgctacgtggagagcgagtcggacctgcggctgcagaactgcgagtcactcacgtccgacagcgccaaggactcggacagcgcaggcgaggcgggcgcgcaggcctccagcaagcaccagaagcgcaagaaaaggcggaaacggcaaaagggcggcagcgccagccgccggcgcctgtccagcgcgtcgagcccaggcggcctggacgcgggcctggtggagcccccgcggctgctgtcctcccccaacagtgcctcggtgctcaagatcaagacggagatctcagaacccatcaatttcgacaatgacagcagcatctggaactacccgcccaaccgggagatctccaggaacgagtccccctacagcatgaccaagccccccagctctgagcacttcccgtccccgcagggcggcggcggtgggggtggcggtggcggggggctgcacgtggccattcccgactcggtcctcaccccgcccggcgccgacggcgcggccgcccgcaagactcagttcggcgcctcggccaccgcggccctggcccccgtcgcctccgacccgctgtcacccccgctctcggcgtccccgcgggacaagcaccccgggaacggcggcgggggcgggggcgggggcggcggcgcggggggcggcggccccagcgcgtccaactccttgctgtacactggggacctggaggcgctgcagaggttgcaggcgggcaacgtcgtgctcccgctggtgcacagggtgaccgggaccctggccgccaccagcacggccgcgcagagggtctacaccacgggcaccatccgctacgcgcccgccgaggtgaccctggccatgcagagcaacctgctgcccaacgcgcacgctgttaacttcgtggacgttaacagccccggctttggcctcgaccccaagacgcccatggagatgctctaccaccacgtgcaccggctcaacatgtcaggaccgttcggcggcgcagtgagcgcagctagcctgacgcagatgcccgccggcaacgtgttcaccacggccgagggactcttctccacgctgcccttccccgtctacagcaacggcatccacgcggcacagactctggagcgcaaggaggactgaggcgccgcccgtcctgggcccggccaggccccgcttggaggaggcatcgtcggcattttcgtttagacctttaattctagcactttgaattcgagcaggtcagcgtcttctctcgccacgacggtccccattccaccccctctttctttcacctgacttattctttcgtgtaaagatatgtttattttttgccttcagagggtcagacgaccagttgcctgccgttttgtcttcttctaaggtgtgtgttgggttgttttgctttcctttgcatctttattaagatgtctttcatgtgtatatgcctctgccatagaatactcagtcttgtggtcaagagagttctcaagtgacaaccattggggtttcttcataaagatcttgatatgatcaagatggaaagagacaagcataaacaatgtgccctgtttgactaagtcaaatgaaatagggtggtttttgtttctgttcctaattcctttaaaaaatagggggaatagtattttagaattttatgcagaatttaattctctttttacggttaagattttaagattttcttacttgcacataaaaataatttgggttcttaaacttaatttctggcctgtgactagaatgtttaaaaaaaaaaaaaaccctcgtgc

SEQ ID NO: 52 - protein, 901 aa, Homo sapiens
MAPTKPSFQQDPSRRERLQALRKEKSRDAARSRRGKENFEFYELAKLLPLPAAITSQLDKASIIRLTISYLKMRDFANQGDPPWNLRMEGPPPNTSVKGAQRRRSPSALAIEVFEAHLGSHILQSLDGFVFALNQEGKFLYISETVSIYLGLSQVELTGSSVFDYVHPGDHVEMAEQLGMKLPPGRGLLSQGTAEDGASSASSSSQSETPEPVESTSPSLLTTDNTLERSFFIRMKSTLTKRGVHIKSSGYKVIHITGRLRLRVSLSHGRTVPSQIMGLVVVAHALPPPTINEVRIDCHMFVTRVNMDLNIIYCENRISDYMDLTPVDIVGKRCYHFIHAEDVEGIRHSHLDLLNKGQCVTKYYRWMQKNGGYIWIQSSATIAINAKNANEKNIIWVNYLLSNPEYKDTPMDIAQLPHLPEKTSESSETSDSESDSKDTSGITEDNENSKSDEKGNQSENSEDPEPDRKKSGNACDNDMNCNDDGHSSSNPDSRDSDDSFEHSDFENPKAGEDGFGALGAMQIKVERYVESESDLRLQNCESLTSDSAKDSDSAGEAGAQASSKHQKRKKRRKRQKGGSASRRRLSSASSPGGLDAGLVEPPRLLSSPNSASVLKIKTEISEPINFDNDSSIWNYPPNREISRNESPYSMTKPPSSEHFPSPQGGGGGGGGGGGLHVAIPDSVLTPPGADGAAARKTQFGASATAALAPVASDPLSPPLSASPRDKHPGNGGGGGGGGGGAGGGGPSASNSLLYTGDLEALQRLQAGNVVLPLVHRVTGTLAATSTAAQRVYTTGTIRYAPAEVTLAMQSNLLPNAHAVNFVDVNSPGFGLDPKTPMEMLYHHVHRLNMSGPFGGAVSAASLTQMPAGNVFTTAEGLFSTLPFPVYSNGIHAAQTLERKED

SEQ ID NO: 53 - DNA, 2961 bp, Homo sapiens
atggggagggccggcgccgcggccaacggcaccccgcagaacgtccagggcatcacctcctaccagcagcgaataactgcccagcatcctctgcccaaccaatcagaatgtaggaaaatctacagatatgacggaatctactgtgaatctacctaccagaatttacaagcattgagaaaggagaaatcccgagatgctgctcgctcccgccggggaaaagaaaactttgagttctatgaattggccaagttgttgcctcttcctgcagccattaccagccagctcgacaaggcatccatcattcgacttacaattagctatctgaaaatgagggactttgctaaccagggggaccctccgtggaacttgcgaatggaaggccctccacctaacacatcagtaaaaggtgcacagcgaaggagaagccccagtgcactagccattgaagtatttgaagcacatttgggaagccacattttgcagtccctggatggctttgtatttgcactaaatcaggaaggaaaatttttgtacatttccgaaacagtctccatctacctaggcctctcacaagtggagctgacaggcagcagtgtctttgactatgtccaccccggagatcacgtggagatggctgagcagctgggcatgaagctcccccctgggcggggtctcctgtcacagggcactgctgaggacggagccagctcagcatcttcctcctctcagtcggagacccccgagccagtggagtcaaccagccccagtctgctaaccactgacaacactcttgagcgttcctttttcatccgaatgaaatctactctgaccaaacgcggtgtgcacatcaaatcatcaggatataaggtgattcacataacaggccggctacgcctgagagtgtcgctgtcccacgggaggaccgtccccagccaaatcatgggtctcgtggttgttgcgcatgccttgcctccccctacgatcaatgaagtcagaattgactgccatatgttcgtcactcgagtaaatatggacctcaatatcatttactgtgaaaataggattagtgattatatggatctgacccctgtagatatcgtagggaagagatgctaccacttcatccatgctgaagacgtggagggcatcaggcacagtcacttggacttgctgaataagggtcagtgtgtgacaaagtactatcgctggatgcagaagaacggaggatatatttggatacagtccagtgccaccatagctattaatgccaagaatgcaaatgaaaagaatatcatctgggtgaattaccttcttagcaatcctgagtacaaggacacacccatggacatcgcacagctcccccatctgccggagaaaacttccgaatcctcggagacatccgactctgagtcagactctaaagacacctcaggtattacagaggacaacgagaactccaagtccgacgagaaggggaaccagtccgagaacagcgaagacccggagcccgaccggaagaagtcgggcaacgcgtgtgacaacgacatgaactgcaacgacgacggccacagctccagtaacccggacagccgcgacagcgacgacagcttcgagcactcggactttgagaaccccaaggcgggcgaggacggcttcggtgctctgggcgcgatgcagatcaaggtggagcgctacgtggagagcgagtcggacctgcggctgcagaactgcgagtcactcacgtccgacagcgccaaggactcggacagcgcaggcgaggcgggcgcgcaggcctccagcaagcaccagaagcgcaagaaaaggcggaaacggcaaaagggcggcagcgccagccgccggcgcctgtccagcgcgtcgagcccaggcggcctggacgcgggcctggtggagcccccgcggctgctgtcctcccccaacagtgcctcggtgctcaagatcaagacggagatctcagaacccatcaatttcgacaatgacagcagcatctggaactacccgcccaaccgggagatctccaggaacgagtccccctacagcatgaccaagccccccagctctgagcacttcccgtccccgcagggcggcggcggtgggggtggcggtggcggggggctgcacgtggccattcccgactcggtcctcaccccgcccggcgccgacggcgcggccgcccgcaagactcagttcggcgcctcggccaccgcggccctggcccccgtcgcctccgacccgctgtcacccccgctctcggcgtccccgcgggacaagcaccccgggaacggcggcgggggcgggggcgggggcggcggcgcggggggcggcggccccagcgcgtccaactccttgctgtacactggggacctggaggcgctgcagaggttgcaggcgggcaacgtcgtgctcccgctggtgcacagggtgaccgggaccctggccgccaccagcacggccgcgcagagggtctacaccacgggcaccatccgctacgcgcccgccgaggtgaccctggccatgcagagcaacctgctgcccaacgcgcacgctgttaacttcgtggacgttaacagccccggctttggcctcgaccccaagacgcccatggagatgctctaccaccacgtgcaccggctcaacatgtcaggaccgttcggcggcgcagtgagcgcagctagcctgacgcagatgcccgccggcaacgtgttcaccacggccgagggactcttctccacgctgcccttccccgtctacagcaacggcatccacgcggcacagactctggagcgcaaggaggactgaggcgccgcccgtcctgggcccggccaggccccgcttggaggaggcatcgtcggcattttcgtttagacctttaattctagcactttgaattcgagcaggtcagcgtcttctctcgccacgacggtccccattccaccccctctt

SEQ ID NO: 54 - protein, 938 aa, Homo sapiens
MGRAGAAANGTPQNVQGITSYQQRITAQHPLPNQSECRKIYRYDGIYCESTYQNLQALRKEKSRDAARSRRGKENFEFYELAKLLPLPAAITSQLDKASIIRLTISYLKMRDFANQGDPPWNLRMEGPPPNTSVKGAQRRRSPSALAIEVFEAHLGSHILQSLDGFVFALNQEGKFLYISETVSIYLGLSQVELTGSSVFDYVHPGDHVEMAEQLGMKLPPGRGLLSQGTAEDGASSASSSSQSETPEPVESTSPSLLTTDNTLERSFFIRMKSTLTKRGVHIKSSGYKVIHITGRLRLRVSLSHGRTVPSQIMGLVVVAHALPPPTINEVRIDCHMFVTRVNMDLNIIYCENRISDYMDLTPVDIVGKRCYHFIHAEDVEGIRHSHLDLLNKGQCVTKYYRWMQKNGGYIWIQSSATIAINAKNANEKNIIWVNYLLSNPEYKDTPMDIAQLPHLPEKTSESSETSDSESDSKDTSGITEDNENSKSDEKGNQSENSEDPEPDRKKSGNACDNDMNCNDDGHSSSNPDSRDSDDSFEHSDFENPKAGEDGFGALGAMQIKVERYVESESDLRLQNCESLTSDSAKDSDSAGEAGAQASSKHQKRKKRRKRQKGGSASRRRLSSASSPGGLDAGLVEPPRLLSSPNSASVLKIKTEISEPINFDNDSSIWNYPPNREISRNESPYSMTKPPSSEHFPSPQGGGGGGGGGGGLHVAIPDSVLTPPGADGAAARKTQFGASATAALAPVASDPLSPPLSASPRDKHPGNGGGGGGGGGGAGGGGPSASNSLLYTGDLEALQRLQAGNVVLPLVHRVTGTLAATSTAAQRVYTTGTIRYAPAEVTLAMQSNLLPNAHAVNFVDVNSPGFGLDPKTPMEMLYHHVHRLNMSGPFGGAVSAASLTQMPAGNVFTTAEGLFSTLPFPVYSNGIHAAQTLERKED

SEQ ID NO: 55 - DNA, 3186 bp, Homo sapiens
gcggccgcggcggtgcagcagaggcgcctcgggcaggaggagggcggcttctgcgagggcagcctgaggtattaaaaagtgtcagcaaactgcattgaataacagacatcctaagaggggatattttccacctctataatgaagaaaagcaggagtgtgatgacggtgatggctgatgataatgttaaagattattttgaatgtagcttgagtaaatcctacagttcttccagtaacacacttgggatcgacctctggagagggagaaggtgttgctcaggaaacttacagttaccaccactgtctcaaagacagagtgaaagggcaaggactcctgagggagatggtatttccaggccgaccacactgcctttgacaacgcttccaagcattgctattacaactgtaagccaggagtgctttgatgtggaaaatggcccttccccaggtcggagtccactggatccccaggccagctcttccgctgggctggtacttcacgccacctttcctgggcacagccagcgcagagagtcatttctctacagatcagacagcgactatgacttgtcaccaaaggcgatgtcgagaaactcttctcttccaagcgagcaacacggcgatgacttgattgtaactccttttgcccaggtccttgccagcttgcgaagtgtgagaaacaacttcactatactgacaaaccttcatggtacatctaacaagaggtccccagctgctagtcagcctcctgtctccagagtcaacccacaagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgcaatgcggccgc

SEQ ID NO: 56 - protein, 736 aa, Homo sapiens
MKKSRSVMTVMADDNVKDYFECSLSKSYSSSSNTLGIDLWRGRRCCSGNLQLPPLSQRQSERARTPEGDGISRPTTLPLTTLPSIAITTVSQECFDVENGPSPGRSPLDPQASSSAGLVLHATFPGHSQRRESFLYRSDSDYDLSPKAMSRNSSLPSEQHGDDLIVTPFAQVLASLRSVRNNFTILTNLHGTSNKRSPAASQPPVSRVNPQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDEQNRDCQGLMEKFQFELTLDEEDSEGPEKEGEGHSYFSSTKTLCVIDPENRDSLGETDIDIATEDKSPVDT

SEQ ID NO: 57 - DNA, 2163 bp, Homo sapiens
atgacagcaaaagattcttcaaaggaacttactgcttctgaacctgaggtttgcataaagactttcaaggagcaaatgcatttagaacttgagcttccgagattaccaggaaacagacctacatctcctaaaatttctccacgcagttcaccaaggaactcaccatgctttttcagaaagttactggtgaataaaagcattcggcagcgtcgtcgcttcactgtggctcatacatgctttgatgtggaaaatggcccttccccaggtcggagtccactggatccccaggccagctcttccgctgggctggtacttcacgccacctttcctgggcacagccagcgcagagagtcatttctctacagatcagacagcgactatgacttgtcaccaaaggcgatgtcgagaaactcttctcttccaagcgagcaacacggcgatgacttgattgtaactccttttgcccaggtccttgccagcttgcgaagtgtgagaaacaacttcactatactgacaaaccttcatggtacatctaacaagaggtccccagctgctagtcagcctcctgtctccagagtcaacccacaagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggataca

SEQ ID NO: 58 - protein, 721 aa, Homo sapiens
MTAKDSSKELTASEPEVCIKTFKEQMHLELELPRLPGNRPTSPKISPRSSPRNSPCFFRKLLVNKSIRQRRRFTVAHTCFDVENGPSPGRSPLDPQASSSAGLVLHATFPGHSQRRESFLYRSDSDYDLSPKAMSRNSSLPSEQHGDDLIVTPFAQVLASLRSVRNNFTILTNLHGTSNKRSPAASQPPVSRVNPQEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDEQNRDCQGLMEKFQFELTLDEEDSEGPEKEGEGHSYFSSTKTLCVIDPENRDSLGETDIDIATEDKSPVDT

SEQ ID NO: 59 - DNA, 4068 bp, Homo sapiens
gaattcctcctctcttcaccccgttagctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttcacccgaattttgactgtttcatttagaagaaaagcaaaatgagaaaaagctttcctcatttctccttgagatggcaaagcactcagaaatgacatcacataccctaaagaaccctgggatgactaaggcagagagagtctgagaaaactctttggtgcttctgcctttagttttaggacacatttatgcagatgagcttataagagaccgttccctccgccttcttcctcagaggaagtttcttggtagatcaccgacacctcatccaggcggggggttggggggaaacttggcaccagccatcccaggcagagcaccactgtgatttgttctcctggtggagagagctggaaggaaggagccagcgtgcaaataatgaaggagcacgggggcaccttcagtagcaccggaatcagcggtggtagcggtgactctgctatggacagcctgcagccgctccagcctaactacatgcctgtgtgtttgtttgcagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgccccccacaggagttgctacagtccctggccctgcttcccatcctcctctcttcaccccgttagctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttggaaacaatattctcacattagatactaaatggtttatactgagtcttttacttttgtatagcttgataggggcaggggcaatgggatgtagtttttacccaggttctatccaaatctatgtgggcatgagttgggttataactggatcctactatcattgtggctttggttcaaaaggaaacactacatttgctcacagatgattcttctgattcttctgaatgctcccgaactactgactttgaagaggtagcctcctgcctgccattaagcaggaatgtcatgttccagttcattacaaaagaaaacaataaaacaatgtgaatttttataataaaaaaaaaaaaaaggaattc

SEQ ID NO: 60 - protein, 564 aa, Homo sapiens
MKEHGGTFSSTGISGGSGDSAMDSLQPLQPNYMPVCLFAEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDEQNRDCQGLMEKFQFELTLDEEDSEGPEKEGEGHSYFSSTKTLCVIDPENRDSLGETDIDIATEDKSPVDT

SEQ ID NO: 61 - DNA, 2929 bp, Homo sapiens
agccatttgtgaacctggaggcttgacattcgccagcgcagggccccacaagagaaatttcaatgaaaagaaaagccaatggattgtggtcttagaaaagctgcttagatgatgtctgtttcccgtgctatagacacgtggcagagctgtaagtaaatgctcggcactgcatgatgaattggatggctgcagaccggagacaaaaaaaataattgtctcattttcgtggtgatttgcttaactggtgggaccatgccagaacggctagcggaaatgctcttggatctctggactccattaataatattatggattactcttcccccttgcatttacatggctccgatgaatcagtctcaagttttaatgagtggatcccctttggaactaaacagtctgggtgaagaacagcgaattttgaaccgctccaaaagaggctgggtttggaatcaaatgtttgtcctggaagagttttctggacctgaaccgattcttgttggccggctacacacagacctggatcctgggagcaaaaaaatcaagtatatcctatcaggtgatggagctgggaccatatttcaaataaatgatgtaactggagatatccatgctataaaaagacttgaccgggaggaaaaggctgagtataccctaacagctcaagcagtggactgggagacaagcaaacctctggagcctccttctgaatttattattaaagttcaagacatcaatgacaatgcaccagagtttcttaatggaccctatcatgctactgtgccagaaatgtccattttgggtacatctgtcactaacgtcactgcgaccgacgctgatgacccagtttatggaaacagtgcaaagttggtttatagtatattggaagggcagccttatttttccattgagcctgaaacagctattataaaaactgcccttcccaacatggacagagaagccaaggaggagtacctggttgttatccaagccaaagatatgggtggacactctggtggcctgtctgggaccacgacacttacagtgactcttactgatgttaatgacaatcctccaaaatttgcacagagcctgtatcacttctcagtaccggaagatgtggttcttggcactgcaataggaagggtgaaggccaatgatcaggatattggtgaaaatgcacagtcatcatatgatatcatcgatggagatggaacagcactttttgaaatcacttctgatgcccaggcccaggatggcattataaggctaagaaaacctctggactttgagaccaaaaaatcctatacgctaaaggtagaggcagccaatgtccatattgacccacgcttcagtggcagggggccctttaaagacacggcgacagtcaaaatcgtggttgaagatgctgatgagcctccggtcttctcttcaccgacttacctacttgaagttcatgaaaatgctgctctaaactccgtgattgggcaagtgactgctcgtgaccctgatatcacttccagtcctataaggttttccatcgaccggcacactgacctggagaggcagttcaacattaatgcagacgatgggaagataacgctggcaacaccacttgacagagaattaagtgtatggcacaacataacaatcattgctactgaaattaggaaccacagtcagatatcacgagtacctgttgctattaaagtgctggatgtcaatgacaacgcccctgaattcgcatccgaatatgaggcatttttatgtgaaaatggaaaacccggccaagtcattcaaactgttagcgccatggacaaagatgatcccaaaaacggacattatttcttatacagtctccttccagaaatggtcaacaatccgaatttcaccatcaagaaaaatgaagataattccctcagtattttggcaaagcataatggattcaaccgccagaagcaagaagtctatcttttaccaatcataatcagtgatagtggaaatcctccactgagcagcactagcaccttgacaatcagggtctgtggctgcagcaatgacggtgtcgtccagtcttgcaatgtcgaagcttatgtccttccaattggactcagtatgggcgccttaattgccatattagcatgcatcattttgctgttagtcatcgtggtgctgtttgtaactctacggcggcataaaaatgaaccattaattatcaaagatgatgaagacgttcgagaaaacatcattcgctacgatgatgaaggaggaggggaggaggacacagaggcttttgacattgcaactttacaaaatccagatggaattaatggatttttaccccgtaaggatattaaaccagatttgcagtttatgccaaggcaagggcttgctccagttccaaatggtgttgatgtcgatgaatttataaatgtaaggctgcatgaggcagataatgatcccacggccccgccatatgactccattcagatatatggctatgaaggccgagggtcagtggctggctccctcagctccttggagtccaccacatcagactcagaccagaattttgactacctcagtgactggggtccccgctttaagagactgggcgaactctactctgttggtgaaagtgacaaagaaacttgacagtggattataaataaatcactggaactgagcattctgtaatattctagggtcactccccttagatacaaccaatgtggctatttgttttagaggcaagtttagcaccagtcatctataaactcaaccacattttaatgttgaaccaaaaaaagataataaaataaaaaagtatatgttaggaggttataaatcttgtggagtgtgaattaagtatgtggagtgtctagaagtccttggatatttgatatttacctgaccaccacagacaaagatt

SEQ ID NO: 62 - protein, 799 aa, Homo sapiens
MPERLAEMLLDLWTPLIILWITLPPCIYMAPMNQSQVLMSGSPLELNSLGEEQRILNRSKRGWVWNQMFVLEEFSGPEPILVGRLHTDLDPGSKKIKYILSGDGAGTIFQINDVTGDIHAIKRLDREEKAEYTLTAQAVDWETSKPLEPPSEFIIKVQDINDNAPEFLNGPYHATVPEMSILGTSVTNVTATDADDPVYGNSAKLVYSILEGQPYFSIEPETAIIKTALPNMDREAKEEYLVVIQAKDMGGHSGGLSGTTTLTVTLTDVNDNPPKFAQSLYHFSVPEDVVLGTAIGRVKANDQDIGENAQSSYDIIDGDGTALFEITSDAQAQDGIIRLRKPLDFETKKSYTLKVEAANVHIDPRFSGRGPFKDTATVKIVVEDADEPPVFSSPTYLLEVHENAALNSVIGQVTARDPDITSSPIRFSIDRHTDLERQFNINADDGKITLATPLDRELSVWHNITIIATEIRNHSQISRVPVAIKVLDVNDNAPEFASEYEAFLCENGKPGQVIQTVSAMDKDDPKNGHYFLYSLLPEMVNNPNFTIKKNEDNSLSILAKHNGFNRQKQEVYLLPIIISDSGNPPLSSTSTLTIRVCGCSNDGVVQSCNVEAYVLPIGLSMGALIAILACIILLLVIVVLFVTLRRHKNEPLIIKDDEDVRENIIRYDDEGGGEEDTEAFDIATLQNPDGINGFLPRKDIKPDLQFMPRQGLAPVPNGVDVDEFINVRLHEADNDPTAPPYDSIQIYGYEGRGSVAGSLSSLESTTSDSDQNFDYLSDWGPRFKRLGELYSVGESDKET

SEQ ID NO: 63 - protein, 93 aa, Homo sapiens
MPERLAEMLLDLWTPLIILWITLPPCIYMAPMNQSQVLMSGSPLELNSLGEEQRILNRSKRGWVWNQMFVLEEFSGPEPILVGRVLKSVSKLH

SEQ ID NO: 64 - DNA, 1062 bp, Homo sapiens
ggccgcggcggtgcagcagaggcgcctcgggcaggaggagggcggcttctgcgagggcagcctgagctacacacagacctggatcctgggagcaaaaaaatcaagtatatcctatcaggtgatggagctgggaccatatttcaaataaatgatgtaactggagatatccatgctataaaaagacttgaccgggaggaaaaggctgagtataccctaacagctcaagcagtggactgggagacaagcaaacctctggagcctccttctgaatttattattaaagttcaagacatcaatgacaatgcaccagagtttcttaatggaccctatcatgctactgtgccagaaatgtccattttgggtacatctgtcactaacgtcactgcgaccgacgctgatgacccagtttatggaaacagtgcaaagttggtttatagtatattggaagggcagccttatttttccattgagcctgaaacagctattataaaaactgcccttcccaacatggacagagaagccaaggaggagtacctggttgttatccaagccaaagatatgggtggacactctggtggcctgtctgggaccacgacacttacagtgactcttactgatgttaatgacaatcctccaaaatttgcacagagcctgtatcacttctcagtaccggaagatgtggttcttggcactgcaataggaagggtgaaggccaatgatcaggatattggtgaaaatgcacagtcatcatatgatatcatcgatggagatggaacagcactttttgaaatcacttctgatgcccaggcccaggatggcattataaggctaagaaaacctctggactttgagaccaaaaaatcctatacgctaaaggtagaggcagccaatgtccatattgacccacgcttcagtggcagggggccctttaaagacacggcgacagtcaaaatcgtggttgaagatgctgatgagcctccggtcttctcttcaccgacttacctacttgaagttcatgaaaatgctgctctaaactccgtgattgggcaagtgactgctcgt

SEQ ID NO: 65 - protein, 354 aa, Homo sapiens
GRGGAAEAPRAGGGRLLRGQPELHTDLDPGSKKIKYILSGDGAGTIFQINDVTGDIHAIKRLDREEKAEYTLTAQAVDWETSKPLEPPSEFIIKVQDINDNAPEFLNGPYHATVPEMSILGTSVTNVTATDADDPVYGNSAKLVYSILEGQPYFSIEPETAIIKTALPNMDREAKEEYLVVIQAKDMGGHSGGLSGTTTLTVTLTDVNDNPPKFAQSLYHFSVPEDVVLGTAIGRVKANDQDIGENAQSSYDIIDGDGTALFEITSDAQAQDGIIRLRKPLDFETKKSYTLKVEAANVHIDPRFSGRGPFKDTATVKIVVEDADEPPVFSSPTYLLEVHENAALNSVIGQVTAR

SEQ ID NO: 66 - DNA, 20 bp, ARTIFICIAL SEQUENCE
  OLIGONUCLEOTIDE
tgccacgtgttagcagaaag

SEQ ID NO: 67 - DNA, 20 bp, ARTIFICIAL SEQUENCE
  OLIGONUCLEOTIDE
tgcctttaaccagatgaggc

SEQ ID NO: 68 - DNA, 21 bp, ARTIFICIAL SEQUENCE
  OLIGONUCLEOTIDE
tcttgtgggtcacaattaggc

SEQ ID NO: 69 - DNA, 23 bp, ARTIFICIAL SEQUENCE
  OLIGONUCLEOTIDE
taaaaaggtgcagtttcttcagc

SEQ ID NO: 70 - DNA, 23 bp, ARTIFICIAL SEQUENCE
  OLIGONUCLEOTIDE
aaatgaatctctgattagccaac

SEQ ID NO: 71 - DNA, 22 bp, ARTIFICIAL SEQUENCE
  OLIGONUCLEOTIDE
tgagagccaacctatttctacc

SEQ ID NO: 72 - DNA, 20 bp, ARTIFICIAL SEQUENCE
  OLIGONUCLEOTIDE
aggctgagtgccaaaaagta

SEQ ID NO: 73 - DNA, 23 bp, ARTIFICIAL SEQUENCE
  OLIGONUCLEOTIDE
ctttaagcttgctatttgaaggc

SEQ ID NO: 74 - DNA, 18 bp, ARTIFICIAL SEQUENCE
  OLIGONUCLEOTIDE
ttcatcgtctgaacctgg

SEQ ID NO: 75 - DNA, 20 bp, ARTIFICIAL SEQUENCE
  OLIGONUCLEOTIDE
acacatttcctctatgttgc

SEQ ID NO: 76 - DNA, 20 bp, ARTIFICIAL SEQUENCE
  OLIGONUCLEOTIDE
caggaggtcctgtgaagctc

SEQ ID NO: 77 - DNA, 20 bp, ARTIFICIAL SEQUENCE
  OLIGONUCLEOTIDE
acagggaaagaagcaaagca

SEQ ID NO: 78 - DNA, 20 bp, ARTIFICIAL SEQUENCE
  OLIGONUCLEOTIDE
aaagctaagcgcaggtgtgt

SEQ ID NO: 79 - DNA, 20 bp, ARTIFICIAL SEQUENCE
  OLIGONUCLEOTIDE
tttctgggaggcaaccatag

SEQ ID NO: 80 - DNA, 20 bp, ARTIFICIAL SEQUENCE
  OLIGONUCLEOTIDE
gcagagttatgtcatgccca

SEQ ID NO: 81 - DNA, 20 bp, ARTIFICIAL SEQUENCE
  OLIGONUCLEOTIDE
cctgtgcagcactctgatgt

SEQ ID NO: 82 - DNA, 20 bp, ARTIFICIAL SEQUENCE
  OLIGONUCLEOTIDE
ttgaacccaagagaacaggg

SEQ ID NO: 83 - DNA, 20 bp, ARTIFICIAL SEQUENCE
  OLIGONUCLEOTIDE
tccccttctccttccagttt

SEQ ID NO: 84 - DNA, 20 bp, ARTIFICIAL SEQUENCE
  OLIGONUCLEOTIDE
acaaccattctgggaacagc

SEQ ID NO: 85 - DNA, 20 bp, ARTIFICIAL SEQUENCE
  OLIGONUCLEOTIDE
gtgtagggaaagccatccaa

SEQ ID NO: 86 - DNA, 20 bp, ARTIFICIAL SEQUENCE
  OLIGONUCLEOTIDE
tctttttcctgcagtccctg

SEQ ID NO: 87 - DNA, 20 bp, ARTIFICIAL SEQUENCE
  OLIGONUCLEOTIDE
ctccaaatgactcctgccat

SEQ ID NO: 88 - DNA, 20 bp, ARTIFICIAL SEQUENCE
  OLIGONUCLEOTIDE
gcctctgccatagattttgc

SEQ ID NO: 89 - DNA, 20 bp, ARTIFICIAL SEQUENCE
  OLIGONUCLEOTIDE
ttccttcccaccctttctct

SEQ ID NO: 90 - DNA, 20 bp, ARTIFICIAL SEQUENCE
  OLIGONUCLEOTIDE
ccagcttgtatgtggtgtgg

SEQ ID NO: 91 - DNA, 20 bp, ARTIFICIAL SEQUENCE
  OLIGONUCLEOTIDE
ttactcccagtgcccattgt

SEQ ID NO: 92 - DNA, 23 bp, ARTIFICIAL SEQUENCE
  OLIGONUCLEOTIDE
gtcagacaaatccaaatggagag

SEQ ID NO: 93 - DNA, 23 bp, ARTIFICIAL SEQUENCE
  OLIGONUCLEOTIDE
ctttctcctgtcactttccttca

SEQ ID NO: 94 - DNA, 9 bp, ARTIFICIAL SEQUENCE
  KOZAK CONSENSUS SEQUENCE
ccannatgg